(19) United States
(12) Patent Application Publication
(10) Pub. No.: US 2024/0010753 A1
(43) Pub. Date: Jan. 11, 2024

(54) ANTI-ROR1 ANTIBODIES AND RELATED BISPECIFIC BINDING PROTEINS

(71) Applicant: EPIMAB BIOTHERAPEUTICS (HK) LIMITED, Hong Kong (CN)

(72) Inventors: Shiyong Gong, Shanghai (CN); Kedong OUYANG, Shanghai (CN); Chengbin Wu, Shanghai (CN); Danqing Wu, Shanghai (CN); Xuan Wu, Shanghai (CN); Rui Zhang, Shanghai (CN)

(21) Appl. No.: 18/042,762

(22) PCT Filed: Aug. 23, 2021
(86) PCT No.: PCT/CN2021/114088

§ 371 Date: Feb. 23, 2023

(30) Foreign Application Priority Data
Aug. 24, 2020 (WO) PCT/CN2020/110841
Dec. 30, 2020 (WO) PCT/CN2020/141398

(51) Int. Cl. C07K 16/46; C12N 15/63; A61P 35/00

(52) U.S. Cl. CPC C07K 16/468; C12N 15/63; A61P 35/00; C07K 2317/565; C07K 2317/24; C07K 2317/31; C07K 2317/55; C07K 2317/522

(57) ABSTRACT

Provided herein are antibodies recognizing receptor tyrosine kinase-like orphan receptor 1 (ROR1), bispecific ROR1/CD3 binding proteins such as FIT-Ig and MAT-Fab binding proteins, and the use of the antibodies and bispecific binding proteins for treating hematopoietic cancers and solid tumors.

### TECHNICAL FIELD

The present disclosure relates to antibodies capable of recognizing the receptor tyrosine kinase-like orphan receptor 1 (ROR1), and to related bispecific binding proteins such as bispecific ROR1/CD3 binding proteins (e.g., FIT-Ig and MAT-Fab binding proteins). The antibodies and bispecific binding proteins disclosed herein may be useful for treating diseases such as hematopoietic cancers and solid tumors.

### BACKGROUND ART

The receptor tyrosine kinase-like orphan receptor 1 (ROR1) is an evolutionarily conserved type I membrane protein that belongs to the ROR subfamily. It shares 58% amino acid (aa) sequence identity with ROR2, the only other member of the ROR family. ROR1 and ROR2 are composed of a distinguished extracellular region with one immunoglobulin like (Ig-like) domain, one frizzled (Fz) domain, and one kringle (Kr) domain, followed by a transmembrane region and an intracellular region containing a tyrosine kinase domain (Baskar, S., et al, (2008) Clinical Cancer Research, 14(2), 396-404).

The expression of ROR1 is developmentally regulated, which attenuates during fetal development. Gene expression profiling of B-cell malignancies and normal B lymphocytes led to the discovery of ROR1 and its distinctive expression in lymphocytic leukemia cells (see, Baskar et al., 2008, supra). By using a high sensitivity murine anti-human ROR1 mAb 6D4, ROR1 was characterized as typically membranous and homogeneously expressed in certain types of solid tumors, including ovarian cancer, triple negative breast cancer, lung adenocarcinomas and pancreatic adenocarcinomas. In addition, cell surface expression of ROR1 was observed in certain normal tissues (e.g., parathyroid, pancreatic islets, and several regions of the human gut), but not in others (e.g., brain, heart, lung, and liver) (Berger et al., 2016, Clinical Cancer Research, 23(12), 3061-3071).

ROR1 has been proposed as a target for cancer treatment. For example, WO2005100605, WO2007051077, WO2008103849 and WO2012097313 described antibodies against ROR1 and their use as therapeutics for targeting tumors, including solid tumors such as breast cancer, and hematological tumors such as chronic lymphocytic leukemia (CLL). Cirmtuzumab, generated by mapping the epitope bound by the anti-ROR1 antibody D10 of WO2012097313, is a humanised monoclonal antibody in clinical trials for various cancers including chronic lymphocytic leukemia (CLL). Cirmtuzumab blocks ROR1 from binding to its ligand Wnt5a, which can inhibit Wnt5a induced stimulation of NF-κB activation and thereby repress autocrine IL-6-dependent STAT3-activation in CLL (Chen et al., 2019, Blood, 134(13), 1084-1094). Cirmtuzumab can internalize into cells, and has been evaluated for use as the targeting moiety in anti-ROR1 antibody drug conjugates (ADCs). A cirmtuzumab-based, MMAE-containing ADC, VLS-101, has been developed for the treatment of patients with ROR1-positive malignancies.

Bispecific antibodies against ROR1 and a second antigen, for instance bispecific T cell engagers (BiTEs), have been developed as another therapeutic modality. WO2014/167022 discloses a bispecific antibody with a slowly internalized anti-ROR1 antibody, R12, as one arm and with an anti-CD3c antibody as another arm. Gohil et al., 2017 (Onco Immunology, 6(7), 1-11) used single chain variable fragments (scFv) targeting the Frizzled domain of ROR1 to generate BiTEs, which prevented engraftment of pancreatic tumor xenografts in murine models. Qi et al., 2018 (Proceedings of the National Academy of Sciences of the United States of America, 115(24), E5467-E5476) discloses an ROR1-targeting scFv with a membrane-proximal epitope, R11, which revealed potent and selective antitumor activity when it was constructed in a scFv-Fc format using an ROR1×CD3 bispecific antibody based on a heterodimeric and aglycosylated Fc domain.

BiTEs are bispecific antibodies directed against a constant-component of the T-cell/CD3 complex and a tumor-associated antigen (TAA). These bispecific antibodies have certain advantages, such as redirecting the cytotoxic activity of T-cells towards malignant cells in a non-MHC restricted fashion. With the clinical success observed with blinatumomab in recent years, there has been a growing interest in CD3-targeting BiTEs for cancer immunotherapy. However, challenges have emerged related to the efficacy and toxicity/safety of this therapeutic modality.

For example, for antigens that are strictly tumor-specific, it may be desirable to have an antibody with an increased affinity. However, for a tumor-associated antigen that is overexpressed in tumors but is also expressed in normal tissues, the ability of an antibody to discriminate between antigen expression in tumors and in normal tissues may be advantageous. The internalization properties of an antibody may also have an impact on its therapeutic application. Strong internalization upon antibody binding, for instance, may be desirable for antibody conjugates to efficiently deliver a conjugated toxin into target cells. However, internalization may be unfavorable for T cell engagers, in that keeping the BiTE at the cell surface may be desirable for eliciting cytotoxic activity by the engagement of T cells. Furthermore, it has been shown that solid tumor penetration and efficacy of antibody drugs may be influenced by the affinity and antigen internalization of the antibody. According to Rudnick et al, 2011 (Rudnick et al., Cancer Res; 71(6); 2250-9), high affinity and rapid internalization may limit penetration of an antibody into a tumor, while a relatively lower affinity and lower internalization may lead to more effective penetration into solid tumors.

Many factors have been mentioned to influence in vivo potency and tumor selectivity of BiTEs in the art. And often, depending on the nature of the target/epitope, it may be desirable to adopt different attributes for a T cell engager.

James et al. (Antigen sensitivity of CD22-specific chimeric TCR is modulated by target epitope distance from the cell membrane, J. Immunol. 180 (10) (2008) 7028-7038), for instance, described modulating epitope distance to the membrane to enhance efficacy and/or tumor selectivity of a BiTE. By targeting an epitope of CD22 with various distances to the membrance with CAR-T cells, James et al. found that targeting an intermediate domain led to efficient lysis of target B cell lines while lysis of normal B cells was undetectable. Similarly, Qi et al., found that epitope location on ROR1 can affect the activity of ROR1×CD3 bispecific antibodies in scFv-Fc format (see, Qi et al., 2018, supra). By screening a panel of mAbs with different epitopes on ROR1, the data of Qi et al. suggest that a membrane-proximal epitope in the Kr domain of ROR1 targeted by R11 may be a suitable site for T cell engagement by bispecific antibodies, while a membrane-distal epitope at the junction of the Fz and Kr domains targeted by R12 may not. A bispecific antibody with an antibody R12 arm revealed only weak in vivo activity against tumors.

Different approaches to increasing the preferential engagement of tumor cells by engineering the antibody format, including the size, valencies and geometries, have been described. Slaga et al. (Avidity-based binding to HER2 results inselective killing of HER2-overexpressing cells by anti-HER2/CD3, Sci. Transl. Med. 10 (463) (2018).) explored an avidity-based strategy in a multivalent antibody format, and developed a bispecific antibody with affinities selected to increase the discrimination between cells expressing HER2 at low or high density. G. L. Moore, et al. (A robust heterodimeric Fc platform engineered for efficient development of bispecific antibodies of multiple formats, Methods (2018)) reported a similar strategy.

Moreover, an issue to be considered in the development of a bispecific antibody is suitability for manufacturing. Low production yields and significant aggregate formation are properties that can render an antibody drug impractical for conducting pre-clinical and clinical stage assessments.

In light of the above, and given that ROR1 is a promising target in cancer treatment, there remains a need in the art to develop diversified anti-ROR1 molecules with different binding potency and/or binding sites or internalization properties, to develop diversified antibody formats, and to expand and/or improve therapeutic utility and suitability for manufacturing.

### SUMMARY

This disclosure addresses the above needs by providing novel anti-ROR1 antibodies, anti-CD3 antibodies, and engineered bispecific proteins that bind both ROR1 and CD3.

In particular, in some embodiments, the present disclosure provides anti-ROR1 antibodies, e.g., those with high binding potency to ROR1-expressing cells and with a low rate of internalization. In some embodiments, the present disclosure also provides antibodies that bind to CD3, e.g., those that bind to CD3 with high affinity. In some embodiments, the present disclosure also provides an ROR1/CD3 bispecific binding protein, in the format of Fabs-in-Tandem immunoglobulin (FIT-Ig) or the format of monovalent asymmetric tandem Fab bispcific antibody (MAT-Fab), that is reactive with both ROR1 and CD3. In some embodiments, antibodies of the present disclosure are useful to detect human ROR1 or human CD3, to inhibit ROR1 signaling, and/or to suppress human ROR1-mediated tumor growth or metastasis, all either in vitro or in vivo. Additionally, in some embodiments, the bispecifc multivalent binding proteins described herein are useful to induce ROR1-redirected T cell cytoxocity and/or in vivo potent anti-tumor activity against ROR1-expressing malignant cells.

In some embodiments, the present disclosure also provides methods of making and using the anti-ROR1 and anti-CD3 antibodies and ROR1/CD3 bispecific binding proteins described herein. Various compositions, e.g., those that may be used in methods of detecting ROR1 and/or CD3 in a sample or in methods of treating or preventing a disorder in an individual that is associated with ROR1 and/or CD3 activity, are also disclosed.

### BRIEF DESCRIPTION OF THE DRAWINGS

FIG. 1 shows the ROR1-ECD protein binding activities of monoclonal antibodies. An irrelevant mIgG1 was used as negative control.

FIGS. 2A-B illustrate the binding activities of anti-ROR1 monoclonal antibodies to ROR1-expressing cells. An irrelevant mIgG1 was used as negative control.

FIG. 3 shows the CD3 binding potency of ROR1×CD3 bispecifics in comparison with their correspondent parental anti-CD3 monoclonal antibodies. An irrelevant hIgG was used as negative control.

FIGS. 4A-D illustrate the ROR1 binding potency of ROR1×CD3 bispecifics and their shared parental anti-ROR1 monoclonal IgG1 antibody (HuROR1-mAb004-1) against ROR1-expressing tumor cells, (A) NCI-H1975, (B) MDA-MB-231, (C) A549 and (D) RPMI-8226.

FIG. 5 shows the results of a co-cultured reporter gene assay measuring redirected CD3 activation by ROR1×CD3 bispecific FIT-Ig and MAT-Fab antibodies, in comparison with monospecific anti-CD3 IgGs (HuEM1006-01-24 and HuEM1006-01-27) and an irrelevant FIT-Ig (EMB01).

FIG. 6 shows the results of a Jurkat-NFAT-luc based reporter gene assay testing the non-target redirected CD3 activation by humanized ROR1×CD3 bispecifics exposure, in comparison with monospecific anti-CD3 IgGs (HuEM1006-01-24 and HuEM1006-01-27) and an irrelevant FIT-Ig (EMB01).

FIG. 7 shows the results of a redirected T cell cytotoxicity assay investigating various ROR1×CD3 bispecifics. An irrelevant FIT-Ig (EMB01) was used as a negative control.

FIG. 8 shows the profile of MDA-MB-231 tumor volume in human PBMC engrafted M-NSG mice treated with ROR1×CD3 bispecific antibodies or vehicle control.

FIGS. 9A-C show the results of internalization assay using humanized anti-ROR1 antibody and bispecific antibodies, (A) HuROR-mAb004-1, (B) FIT1007-12B-17, and (C) MAT1007-12B-17.

FIG. 10A provides schematic illustration of the domain structure of a FIT-Ig bispecific antibody, in Format LH and Format HL. FIG. 10B provides schematic illustration of the domain structure of a MAT-Fab bispecific antibody, in Format LH and Format HL.

FIG. 11A shows the cell binding activity of FIT-Ig molecules to ROR1 expressing MDA-MB-231 cells. FIG. 11B shows the cell binding activity of FIT-Ig molecules to CD3 expressing Jurkat cells. FIG. 11C shows the results of a redirected T cell cytotoxicity assay to compare FIT1007-12B-17 with two reference FIT-Ig molecules.

### DETAILED DESCRIPTION

This present disclosure pertains to anti-ROR1 antibodies, anti-CD3 antibodies, antigen-binding portions thereof, and multivalent, bispecific binding proteins such as FIT-Igs or MAT-Fabs that bind to both ROR1 and CD3. Various aspects of the present disclosure relate to anti-ROR1 and anti-CD3 antibodies and antibody fragments, FIT-Ig and MAT-Fab binding proteins that bind to human ROR1 and human CD3, and pharmaceutical compositions thereof, as well as nucleic acids, recombinant expression vectors and host cells for making such antibodies, functional antibody fragments, and binding proteins. Methods of using the antibodies, functional antibody fragments, and bispecific binding proteins of the present disclosure to detect human ROR1, human CD3, or both; to modulate human ROR1 and/or human CD3 activity, either in vitro or in vivo; and to treat diseases, especially cancer, that are mediated by ROR1 and CD3 binding to their respective ligands, are also encompassed by the present disclosure.

### Definitions

Unless otherwise defined herein, scientific and technical terms used in connection with the present disclosure shall have the meanings that are commonly understood by those of ordinary skill in the art. In the event of any latent ambiguity, definitions provided herein take precedent over any dictionary or extrinsic definition. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CHL Hinge, CH2 and CH3, which is herein further clarified by referring to “numbering according to Kabat EU index” in this case).

General information regarding the sequences of human immunoglobulins light and heavy chains is given in: Kabat, E. A., et al., Sequences of Proteins of Immunological Interest, 5th ed., Public Health Service, National Institutes of Health, Bethesda, MD (1991).

The term “isolated protein” or “isolated polypeptide” is a protein or polypeptide that by virtue of its origin or source of derivation is not associated with naturally associated components that accompany it in its native state, is substantially free of other proteins from the same species, is expressed by a cell from a different species, or does not occur in nature. A polypeptide that is chemically synthesized or synthesized in a cellular system different from the cell from which it naturally originates may be “isolated” from its naturally associated components. A protein may also be rendered substantially free of naturally associated components by isolation, using protein purification techniques well known in the art.

The term “specific binding” or “specifically binding” in reference to the interaction of an antibody, a binding protein, or a peptide with a second chemical species, means that the interaction is dependent upon the presence of a particular structure (e.g., an antigenic determinant or epitope) on the second chemical species. For example, an antibody recognizes and binds to a specific protein structure rather than to proteins generally. In general, if an antibody is specific for epitope “A”, the presence of a molecule containing epitope A (or free, unlabeled A), in a reaction containing labeled “A” and the antibody, will reduce the amount of labeled A bound to the antibody.

The term “antibody” broadly refers to any immunoglobulin (Ig) molecule comprised of four polypeptide chains, two heavy (H) chains and two light (L) chains, or any functional fragment, mutant, variant, or derivation thereof, which retains the essential epitope binding features of an Ig molecule. Such mutant, variant, or derivative antibody formats are known in the art and non-limiting embodiments are discussed below.

In a full-length antibody, each heavy chain is comprised of a heavy chain variable region (abbreviated herein as VH) and a heavy chain constant region. The heavy chain constant region is comprised of three domains: CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FRs). Each VH and VL is comprised of three CDRs and four FRs, arranged from amino-terminus to carboxy-terminus in the following order: FR1, CDR1, FR2, CDR2, FR3, CDR3, FR4. First, second and third CDRs of a VH domain are commonly enumerated as CDR-H1, CDR-H2, and CDR-H3; likewise, first, second and third CDRs of a VL domain are commonly enumerated as CDR-L1, CDR-L2, and CDR-L3. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “Fc region” is used to define the C-terminal region of an immunoglobulin heavy chain, which may be generated by papain digestion of an intact antibody. The Fc region may be a native sequence Fc region or a variant Fc region. The Fc region of an immunoglobulin generally comprises two constant domains, i.e., a CH2 domain and a CH3 domain, and optionally comprises a CH4 domain, for example, as in the case of the Fc regions of IgM and IgE antibodies. The Fc region of IgG, IgA, and IgD antibodies comprises a hinge region, a CH2 domain, and a CH3 domain. In contrast, the Fc region of IgM and IgE antibodies lacks a hinge region but comprises a CH2 domain, a CH3 domain and a CH4 domain. Variant Fc regions having replacements of amino acid residues in the Fc portion to alter antibody effector function are known in the art (see, e.g., Winter et al., U.S. Pat. Nos. 5,648,260 and 5,624,821). The Fc portion of an antibody may mediate one or more effector functions, for example, cytokine induction, ADCC, phagocytosis, complement dependent cytotoxicity (CDC), and/or half-life/clearance rate of antibody and antigen-antibody complexes. In some cases, these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. In still another embodiment at least one amino acid residue is replaced in the constant region of the antibody, for example the Fc region of the antibody, such that effector functions of the antibody are altered. The dimerization of two identical heavy chains of an immunoglobulin is mediated by the dimerization of CH3 domains and is stabilized by the disulfide bonds within the hinge region that connects CH1 constant domains to the Fc constant domains (e.g., CH2 and CH3). The anti-inflammatory activity of IgG is dependent on sialylation of the N-linked glycan of the IgG Fc fragment. The precise glycan requirements for anti-inflammatory activity have been determined, such that an appropriate IgG1 Fc fragment can be created, thereby generating a fully recombinant, sialylated IgG1 Fc with greatly enhanced potency (see, Anthony et al., Science, 320:373-376 (2008)).

The terms “antigen-binding portion” and “antigen-binding fragment” or “functional fragment” of an antibody are used interchangeably and refer to one or more fragments of an antibody that retain the ability to specifically bind to an antigen, i.e., the same antigen (e.g., ROR1, CD3) as the full-length antibody from which the portion or fragment is derived. It has been shown that the antigen-binding function of an antibody can be performed by fragments of a full-length antibody. Such antibody embodiments may also be bispecific, dual specific, or multi-specific formats; specifically binding to two or more different antigens (e.g., ROR1 and a different antigen, such as CD3). Examples of binding fragments encompassed within the term “antigen-binding portion” of an antibody include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) an Fd fragment consisting of the VH and CH1 domains; (iv) a Fv fragment consisting of the VL and VH domains of a single arm of an antibody, (v) a dAb fragment (Ward et al., Nature, 341: 544-546 (1989); PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, for example, Bird et al., Science, 242: 423-426 (1988); and Huston et al., Proc. Natl. Acad. Sci. USA, 85: 5879-5883 (1988)). Such single chain antibodies are also intended to be encompassed within the term “antigen-binding portion” of an antibody and equivalent terms given above. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci. USA, 90: 6444-6448 (1993). Such antibody binding portions are known in the art (Kontermann and Dübel eds., Antibody Engineering (Springer-Verlag, New York, 2001), p. 790 (ISBN 3-540-41354-5)). In addition, single chain antibodies also include “linear antibodies” comprising a pair of tandem Fv segments (VH-CH1-VH-CH1) which, together with complementary light chain polypeptides, form a pair of antigen binding regions (Zapata et al., Protein Eng., 8(10): 1057-1062 (1995); and U.S. Pat. No. 5,641,870)).

An immunoglobulin constant (C) domain refers to a heavy (CH) or light (CL) chain constant domain. Murine and human IgG heavy chain and light chain constant domain amino acid sequences are known in the art.

The term “monoclonal antibody” or “mAb” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic determinant (epitope). Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method.

The term “human sequence”, in relation to the light chain constant domain CL, heavy chain constant domain CH, and Fc region of the antibody or the binding protein according to the present application, means the sequence is of, or from, human immunoglobulin sequence. The human sequence of the present disclosure may be native human sequence, or a variant thereof including one or more (for example, up to 20, 15, 10) amino acid residue changes.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL are replaced with CDR sequences of another species, such as antibodies having human heavy and light chain variable regions in which one or more of the human CDRs has been replaced with murine CDR sequences.

The term “humanized antibody” refers to antibodies that comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which CDR sequences from a non-human species (e.g., mouse) are introduced into human VH and VL framework sequences. A humanized antibody is an antibody or a variant, derivative, analog or fragment thereof which immunospecifically binds to an antigen of interest and which comprises framework regions and constant regions having substantially the amino acid sequence of a human antibody but complementarity determining regions (CDRs) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CHL hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody may be selected from any class of immunoglobulins, including IgM, IgG, IgD, IgA and IgE, and any isotype, including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the acceptor framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, at least 85%, at least 90%, or at least 95% of the humanized antibody residues will correspond to those of the parental FR and CDR sequences. Back mutation at a particular framework position to restore the same amino acid that appears at that position in the donor antibody is often utilized to preserve a particular loop structure or to correctly orient the CDR sequences for contact with target antigen.

The term “CDR” refers to the complementarity determining regions within antibody variable domain sequences. There are three CDRs in each of the variable regions of the heavy chain and the light chain, which are designated CDR-H1, CDR-H2, CDR-H3, CDR-L1, CDR-L2, and CDR-L3. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Maryland (1987) and (1991)) not only provides an unambiguous residue numbering system applicable to any variable region of an antibody, but also provides precise residue boundaries defining the three CDRs.

The term “Kabat numbering”, in relation to heavy and light chain CDRs of an antibody, which is recognized in the art, refers to a system of numbering amino acid residues which are more variable (i.e., hypervariable) than other amino acid residues in the heavy and light chain variable regions of an antibody or an antigen-binding portion thereof. See, Kabat et al., Ann. NY Acad. Sci., 190: 382-391 (1971); and Kabat et al., Sequences of Proteins of Immunological Interest, Fifth Edition, U.S. Department of Health and Human Services, NIH Publication No. 91-3242 (1991).

The growth and analysis of extensive public databases of amino acid sequences of variable heavy and light regions over the past twenty years have led to the understanding of the typical boundaries between framework regions (FRs) and CDR sequences within variable region sequences and have enabled persons skilled in the art to accurately determine the CDRs according to Kabat numbering, Chothia numbering, or other systems. See, e.g., Martin, “Protein Sequence and Structure Analysis of Antibody Variable Domains,” In Kontermann and Dübel, eds., Antibody Engineering (Springer-Verlag, Berlin, 2001), chapter 31, pages 432-433.

The term “multivalent binding protein” denotes a binding protein comprising two or more antigen binding sites. A multivalent binding protein is, in certain cases, engineered to have three or more antigen binding sites, and is generally not a naturally occurring antibody. The term “bispecific binding protein” (which can be used interchangeably with the term “bispecific antibody”, unless stated otherwise) refers to a binding protein capable of binding two targets of different specificity. FIT-Ig binding proteins of the present disclosure comprise four antigen binding sites and are typically tetravalent binding proteins. MAT-Fab binding proteins of the present disclosure comprise two antigen binding sites and are typically bivalent binding proteins. A FIT-Ig or MAT-Fab according to this disclosure binds both ROR1 and CD3 and is bispecific.

A FIT-Ig binding protein comprising two long (heavy) V-C-V-C-Fc chain polypeptides and four short (light) V-C chain polypeptides forms a hexamer exhibiting four Fab antigen binding sites (VH-CH1 paired with VL-CL, sometimes notated VH-CH1::VL-CL). Each half of a FIT-Ig comprises a heavy chain polypeptide and two light chain polypeptides, and complementary immunoglobulin pairing of the VH-CH1 and VL-CL elements of the three chains results in two Fab-structured antigen binding sites, arranged in tandem. In the present disclosure, it is preferred that the immunoglobulin domains comprising the Fab elements are directly fused in the heavy chain polypeptide, without the use of interdomain linkers. That is, the N-terminal V-C element of the long (heavy) polypeptide chains is directly fused at its C-terminus to the N-terminus of another V-C element, which in turn is linked to a C-terminal Fc region. In bispecific FIT-Ig binding proteins, the tandem Fab elements may be reactive with different antigens. Each Fab antigen binding site comprises a heavy chain variable domain and a light chain variable domain with a total of six CDRs per antigen binding site.

A description of the design, expression, and characterization of FIT-Ig molecules is provided in PCT Publication WO 2015/103072. An example of such FIT-Ig molecules comprises a heavy chain and two different light chains. The heavy chain comprises the structural formula VL_{A}-CL-VH_{B}-CH1-Fc where CL is directly fused to VH_{B} (namely “Format LH”) or VH_{A}-CH1-VL_{B}-CL-Fc where CH1 is fused directly to VL_{B} (namely “Format HL”), and the two light polypeptide chains of the FIT-Ig correspondingly have the formulas VH_{A}-CH1 and VL_{B}-CL (for “Format LH”) or VL_{A}-CL and VH_{B}-CH1 (for “Format HL”), respectively; wherein VL_{A} is a variable light domain from a parental antibody that binds antigen A, VL_{B} is a variable light domain from a parental antibody that binds antigen B, VH_{A} is a variable heavy domain from a parental antibody that binds antigen A, VH_{B} is a variable heavy domain from a parental antibody that binds antigen B, CL is a light chain constant domain, CH1 is a heavy chain constant domain, and Fc is an immunoglobulin Fc region (e.g., the C-terminal hinge-CH2-CH3 portion of a heavy chain of an IgG1 antibody). In bispecific FIT-Ig embodiments, antigen A and antigen B are different antigens, or different epitopes of the same antigen. In the present disclosure, one of A and B is ROR1 and the other is CD3, for example, A is ROR1 and B is CD3.

A MAT-Fab binding protein comprising one long (heavy)V-C-V-C-Fc chain polypeptide, two short (light) V-C chain polypeptides, and one immunological Fc chain polypeptide forms a tetramer exhibiting two Fab antigen binding sites arranged in tandem (VH-CH1 paired with VL-CL, sometimes notated VH-CH1::VL-CL), and one Fc:Fc dimer. Often modifications have been introduced into the CH3 domain of Fc region of the MAT-Fab heavy chain (abbreviated as CH3m1 domain) and also the CH3 domain of the MAT-Fab Fc polypeptide chain (abbreviated as CH3m2 domain) to favor the heterodimerization of the two CH3 domains. The modifications can be “knob-in-hole” (KIH) mutations, for instance, a mutation is made to form a structural knob in the CH3m1 domain of the heavy chain for pairing with a CH3m2 domain of the Fc chain that comprises a complementary structural hole. However, other modifications such as those introduced into the domains salt bridges or electrostatic interactions are also useful. The constant regions may also other modifications, for example, Cys residues to stable the MAT-Fab molecule, and/or mutations to prevent or impair the Fc effector functions. Preferably, a feature of the structure of a MAT-Fab bispecific antibody described herein is that all adjacent immunoglobulin heavy and light chain variable and constant domains are linked directly to one another without an intervening synthetic amino acid or peptide linker.

A description of the design, expression, and characterization of MAT-Fab molecules is provided in PCT Publication WO2018/035084. One example of such MAT-Fab molecules comprises a heavy chain with a “knob” in Fc region, two different light chains, and one Fc polypeptide chain with a “hole”. In some embodiments, the heavy chain comprises the structural formula VL_{A}-CL-VH_{B}-CH1-hinge-CH2-CH3m1 where CL is directly fused to VH_{B} (namely “Format LH”), or VH_{A}-CH1-VL_{B}-CL-Fc where CH1 is fused directly to VL_{B} (namely “Format HL”), and the two light polypeptide chains of the MAT-Fab correspondingly have the formulas VH_{A}-CH1 and VL_{B}-CL (for “Format LH”) or VL_{A}-CL and VH_{B}-CH1 (for “Format HL”), respectively; wherein VL_{A} is a variable light domain from a parental antibody that binds antigen A, VL_{B} is a variable light domain from a parental antibody that binds antigen B, VH_{A} is a variable heavy domain from a parental antibody that binds antigen A, VH_{B} is a variable heavy domain from a parental antibody that binds antigen B, CL is a light chain constant domain, CH1 is a heavy chain constant domain 1, and CH3m1 is a heavy chain constant domain 3 with knob mutations such as S354C and T366W. The Fc polypeptide chain may be the C-terminal hinge-CH2-CH3 portion of a heavy chain of an immunoglobulin (such as IgG antibody), with hole mutations complementary to knob mutations in CH3m2 such as T366S, L368A, and Y407V. In bispecific MAT-Fab embodiments, antigen A and antigen B are different antigens, or different epitopes of the same antigen. In the present disclosure, one of antigen A and B is ROR1 and the other is CD3, for example, A is ROR1 and B is CD3.

The term “kₒₙ” (also “Kon”, “kon”), as used herein, is intended to refer to the on-rate constant for association of a binding protein (e.g., an antibody) to an antigen to form an association complex, e.g., antibody/antigen complex, as is known in the art. The “kₒₙ” also is known by the terms “association rate constant”, or “ka”, as used interchangeably herein. This value indicates the binding rate of an antibody to its target antigen or the rate of complex formation between an antibody and antigen as is shown by the equation below:

Antibody (“Ab”)+Antigen (“Ag”)→Ab−Ag.

The term “k_{off}” (also “Koff”, “koff”), as used herein, is intended to refer to the off-rate constant for dissociation, or “dissociation rate constant”, of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab−Ag.

The term “K_{D}” (also “K_{d}”), as used herein, is intended to refer to the “equilibrium dissociation constant”, and refers to the value obtained in a titration measurement at equilibrium, or by dividing the dissociation rate constant (k_{off}) by the association rate constant (kₒₙ). The association rate constant (kₒₙ), the dissociation rate constant (k_{off}), and the equilibrium dissociation constant (K_{D}) are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Biolayer interferometry (BLD using, e.g., the Octet® RED96 system (Pall ForteBio LLC), is another affinity assay technique. Additionally, a KinExA® (Kinetic Exclusion Assay) assay, available from Sapidyne Instruments (Boise, Idaho) can also be used.

The term “isolated nucleic acid” means a polynucleotide (e.g., of genomic, cDNA, or synthetic origin, or some combination thereof) that, by human intervention, is not associated with all or a portion of the polynucleotides with which it is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the present disclosure is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequence. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, transfection, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NS0, SP2 and PER.C6; the insect cell line Sf9; and the fungal cell Saccharomyces cerevisiae.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, or progression of one or more symptoms associated with a disorder; detect a disorder; or enhance or improve the prophylactic or therapeutic effect(s) of another therapy (e.g., prophylactic or therapeutic agent).

Antibodies, functional fragments thereof, and binding proteins according to the present disclosure may be purified (for an intended use) by using one or more of a variety of methods and materials available in the art for purifying antibodies and binding proteins. Such methods and materials include, but are not limited to, affinity chromatography (e.g., using resins, particles, or membranes conjugated to Protein A, Protein G, Protein L, or a specific ligand of the antibody, functional fragment thereof, or binding protein), ion exchange chromatography (for example, using ion exchange particles or membranes), hydrophobic interaction chromatography (“HIC”; for example, using hydrophobic particles or membranes), ultrafiltration, nanofiltration, diafiltration, size exclusion chromatography (“SEC”), low pH treatment (to inactivate contaminating viruses), and combinations thereof, to obtain an acceptable purity for an intended use. A non-limiting example of a low pH treatment to inactivate contaminating viruses comprises reducing the pH of a solution or suspension comprising an antibody, functional fragment thereof, or binding protein of the present disclosure to pH 3.5 with 0.5 M phosphoric acid, at 18° C.-25° C., for 60 to 70 minutes.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

### Anti-ROR1 and Anti-CD3 Monospecific Antibodies

Anti-ROR1 and anti-CD3 antibodies of the present disclosure may be produced by any of a number of techniques known in the art. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection, and the like. Although it is possible to express the antibodies of the present disclosure in either prokaryotic or eukaryotic host cells, expression of antibodies in eukaryotic cells, for instance, in mammalian host cells, is particularly contemplated, because such eukaryotic cells (and in particular mammalian cells) are more likely than prokaryotic cells to assemble and secrete a properly folded and immunologically active antibody.

In some embodiments, mammalian host cells for expressing the recombinant antibodies of the present disclosure is Chinese Hamster Ovary (CHO cells) (including dhfr CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982)), NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells, or further secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the present disclosure. For example, it may be desirable to transfect a host cell with DNA encoding functional fragments of either the light chain and/or the heavy chain of an antibody of this disclosure. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the present disclosure. In addition, bifunctional antibodies may be produced in which one heavy and one light chain are an antibody of the present disclosure and the other heavy and light chain are specific for an antigen other than the antigens of interest by crosslinking an antibody of the present disclosure to a second antibody by standard chemical crosslinking methods.

In an exemplary system for recombinant expression of an antibody, or antigen-binding portion thereof, of the present disclosure, a recombinant expression vector encoding both the antibody heavy chain and the antibody light chain is introduced into dhfr CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transfected host cells are cultured to allow for expression of the antibody heavy and light chains and intact antibody is recovered from the culture medium. Standard molecular biology techniques are used to prepare the recombinant expression vector, transfect the host cells, select for transfectants, culture the host cells and recover the antibody from the culture medium. Still further the present disclosure provides a method of making a recombinant anti-ROR1 or anti-CD3 antibody by culturing a transfected host cell of the present disclosure in a suitable culture medium until a recombinant antibody of the present disclosure is produced. The method can further comprise isolating the recombinant antibody from the culture medium.

### Anti-ROR1 Antibodies

In some embodiments, the present disclosure provides antibodies that bind to ROR1 at the C-terminus of the ROR1 Ig-like domain. The antibodies disclosed herein, in some embodiments, have high cell binding potency and/or are characterized by low internalization rate, e.g., as measured in a cell-based assay.

In some embodiments, the present disclosure discloses an isolated anti-ROR1 antibody or antigen-binding fragment thereof that specifically binds to ROR1. In a further embodiment, the anti-ROR1 antibody or antigen-binding fragment thereof comprises a set of six CDRs, CDR-H1, CDR-H2, CDR-H3, CDR-L1, CDR-L2, and CDR-L3, wherein:
CDR-H1 comprises the sequence of RSWMN (SEQ ID NO:1);
CDR-H2 comprises the sequence of RIYPGNGDIKYNGNFKG (SEQ ID NO: 2) or RIYPGNADIKYNANFKG (SEQ ID NO: 4);
CDR-H3 comprises the sequence of IYYDFYYALDY (SEQ ID NO: 3);
CDR-L1 comprises the sequence of KASQDINKYIT (SEQ ID NO: 5);
CDR-L2 comprises the sequence of YTSTLQP (SEQ ID NO: 6);
CDR-L3 comprises the sequence of LQYDSLLWT (SEQ ID NO: 7),
wherein the CDRs are defined according to Kabat numbering.

In some embodiments, the anti-ROR1 antibody or antigen-binding fragment thereof comprises, at positions H31-H35, H50-H65, and H95-H102 according to Kabat numbering, the amino acid sequences of CDR-H1, CDR-H2, and CDR-H3 selected from the group of consisting of: (i) SEQ ID NOs: 1, 2, 3; or (ii) SEQ ID NOs: 1, 4, 3.

In one embodiment, the anti-ROR1 antibody or antigen-binding fragment thereof comprises, at positions L24-34, L50-56 and L89-97 according to Kabat numbering, the amino acid sequences of SEQ ID NOs: 5, 6 and 7 for CDR-L1, CDR-L2, and CDR-L3, respectively.

In certain embodiments, the anti-ROR1 antibody or antigen-binding fragment thereof comprises G55A and G61A mutations in the VH domain according to Kabat numbering. In some embodiments, the mutations reduce the propensity of asparagine deamidation in the anti-ROR1 antibody or antigen-binding fragment thereof. In some embodiments, the anti-ROR1 antibody or antigen-binding fragment thereof with the mutations has increased stability relative to the parental antibody without the mutations.

In some embodiments, the anti-ROR1 antibody or antigen-binding fragment thereof comprises at least one, two, three, four, but not more than five residue modifications in the CDR sequences of SEQ ID NOs: 1-3 and 5-7. In some embodiments, the anti-ROR1 antibody or antigen-binding fragment thereof comprises at least one, two, three, four, but not more than five residue modifications in the CDR sequences of SEQ ID NOs: 1, 4, 3 and 5-7. The amino acid modifications may be amino acid substitutions, deletions, and/or additions, for instance, conservative substitution.

In one embodiment, an anti-ROR1 antibody or antigen-binding fragment thereof according to the present disclosure comprises CDR-H1, CDR-H2, CDR-H3, CDR-L1, CDR-L2, and CDR-L3 of a heavy chain variable domain VH and a light chain variable domain VL, selected from the group consisting of the following VH/VL sequence pairs: SEQ ID NOs: 8/9, 17/9, 10/13, 10/14, 10/15, 10/16, 11/13, 11/14, 11/15, 11/16, 12/13, 12/14, 12/15, 12/16, and 21/13. The CDRs can be determined by a person skilled in the art using the most widely CDR definition schemes, for example, Kabat, Chothia or IMGT definitions.

In one embodiment, an anti-ROR1 antibody or antigen-binding fragment thereof according to the present disclosure comprises a heavy chain variable domain VH and a light chain variable domain VL, wherein:
the VH domain comprises the sequence of SEQ ID NO:8 or 17, or a sequence having at least 80%, 85%, 90%, 91%, 92%, 93%, 94%, 95%, 96%, 97%, 98%, 99% or more identity therewith, and/or
the VL domain comprises the sequence of SEQ ID NO:9, or a sequence having at least 80%, 85%, 90%, 91%, 92%, 93%, 94%, 95%, 96%, 97%, 98%, 99% or more identity therewith.

In another embodiment, an anti-ROR1 antibody or antigen-binding fragment thereof according to the present disclosure comprises a heavy chain variable domain VH and a light chain variable domain VL, wherein:
the VH domain comprises the sequence selected from SEQ ID NOs: 10-12 and 21, or a sequence having at least 80%, 85%, 90%, 91%, 92%, 93%, 94%, 95%, 96%, 97%, 98%, 99% or more identity therewith, and/or
the VL domain comprises the sequence selected from SEQ ID NOs: 13-16, or a sequence having at least 80%, 85%, 90%, 91%, 92%, 93%, 94%, 95%, 96%, 97%, 98%, 99% or more identity therewith.

In some embodiments, an anti-ROR1 antibody comprises a VH sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the ROR1 with the same or improved binding properties, such as the off-rate and/or the internalization rate. In some embodiments, a total of 1 to 10 amino acids have been substituted, inserted and/or deleted in SEQ ID NO: 8, 17, or SEQ ID NO: 10-12 or 21. In certain embodiments, substitutions, insertions, or deletions occur in regions outside the CDRs (i.e., in the FRs). Optionally, the anti-ROR1 antibody comprises the VH sequence of SEQ ID NO: 8, 17, or SEQ ID NO: 10-12 or 21, including post-translational modifications of that sequence. In a particular embodiment, the VH comprises one, two or three CDRs selected from: (a) a CDR-H1 comprising the amino acid sequence of SEQ ID NO: 1, (b) a CDR-H2 comprising the amino acid sequence of SEQ ID NO: 2/4, and (c) a CDR-H3 comprising the amino acid sequence of SEQ ID NO: 3. In some embodiments, the VH sequence is a humanized VH sequence.

In some embodiments, an anti-ROR1 antibody comprises a VL sequence having at least 80%, 85%, 90%, 91%, 92%, 93%, 94%, 95%, 96%, 97%, 98%, or 99% identity contains substitutions (e.g., conservative substitutions), insertions, or deletions relative to the reference sequence, while retains the ability to bind to the ROR1 with the same or improved binding properties, such as the off-rate and/or the internalization rate. In some embodiments, a total of 1 to 10 amino acids have been substituted, inserted and/or deleted in SEQ ID NO: 13. In certain embodiments, substitutions, insertions, or deletions occur in regions outside the CDRs (i.e., in the FRs). Optionally, the anti-ROR1 antibody comprises the VL sequence of SEQ ID NO: 13, including post-translational modifications of that sequence. In a particular embodiment, the VL sequence comprises one, two or three CDRs selected from: (a) a CDR-L1 comprising the amino acid sequence of SEQ ID NO: 5, (b) a CDR-L2 comprising the amino acid sequence of SEQ ID NO: 6, and (c) a CDR-L3 comprising the amino acid sequence of SEQ ID NO: 7. In some embodiments, the VL sequence is a humanized VL sequence.

In one embodiment, an anti-ROR1 antibody or antigen-binding fragment thereof according to the present disclosure comprises a heavy chain variable domain VH comprising or consisting of SEQ ID NO: 21, and a light chain variable domain VL comprising or consisting of SEQ ID NO: 13.

In one embodiment, the isolated anti-ROR1 antibody or antigen-binding fragment according to the present disclosure is a chimeric antibody or a humanized antibody. In some embodiments, the anti-ROR1 antibody or antigen-binding fragment is a humanized antibody.

In some embodiments, the humanized isolated anti-ROR1 antibody or antigen-binding fragment according to the present disclosure comprises one or more back mutations at positions in framework regions to improve the binding property. In some embodiments, the VH domain of the humanized anti-ROR1 antibody or antigen-binding fragment according to the present disclosure comprises back mutations from human to residues: a Glu at position 1 (1E), a Tyr at position 27 (27Y), a His at position 94 (94H), and optionally one or more of a Lys at position 38 (38K), an Ile at position 48 (481), a Lys at position (66K), and an Ala at position 67 (67A), according to Kabat numbering. In one embodiment, the VL domain of the humanized anti-ROR1 antibody or antigen-binding fragment according to the present disclosure comprises back mutations from human to residues: a Tyr at position 71 (71Y), and optionally one or more of a Leu at position 4 (4L), an Arg at position 69 (69R), a His at position 49 (49H), an Ile at position 58 (581), according to Kabat numbering.

In one embodiment, the isolated anti-ROR1 antibody or antigen-binding fragment according to the present disclosure is a humanized antibody, comprising back-mutated amino acid residues in the VH domain selected from the group consisting of: (i) 1E, 27Y, and 94H, (ii) 1E, 27Y, 481, 67A, and 94H, (iii) 1E, 27Y, 38K, 481, 67A, 66K, and 94H, all according to Kabat numbering; and/or back-mutated amino acid residues in the VL domain selected from the group consisting of: (i) 71Y; (ii) 49H, 69R, and 71Y, (iii) 4L, 69R, and 71Y, and (iv) 4L, 49H, 581, 69R, and 71Y, all according to Kabat numbering.

In one embodiment, the isolated anti-ROR1 antibody or antigen-binding fragment according to the present disclosure is a humanized antibody, comprising amino acid residues 1E, 27Y, and 94H in the VH domain, and amino acid residue 71Y in the VL domain, according to Kabat numbering. In a further embodiment, the isolated anti-ROR1 antibody or antigen-binding fragment according to the present disclosure further comprises G55A and G61A mutations in the VH domain according to Kabat numbering.

In some embodiments, the isolated anti-ROR1 antibody or antigen-binding fragment according to the present disclosure comprises a combination of VH and VL sequences selected from the group consisting of:

| | VH sequence, | VL sequence, |
|---|---|---|
| | which comprises | which comprises |
| combination | or consists of | or consists of |
| 1 | SEQ ID NO: 8 | SEQ ID NO: 9 |
| 2 | SEQ ID NO: 17 | SEQ ID NO: 9 |
| 3 | SEQ ID NO: 10 | SEQ ID NO: 13 |
| 4 | SEQ ID NO: 10 | SEQ ID NO: 14 |
| 5 | SEQ ID NO: 10 | SEQ ID NO: 15 |
| 6 | SEQ ID NO: 10 | SEQ ID NO: 16 |
| 7 | SEQ ID NO: 11 | SEQ ID NO: 13 |
| 8 | SEQ ID NO: 11 | SEQ ID NO: 14 |
| 9 | SEQ ID NO: 11 | SEQ ID NO: 15 |
| 10 | SEQ ID NO: 11 | SEQ ID NO: 16 |
| 11 | SEQ ID NO: 12 | SEQ ID NO: 13 |
| 12 | SEQ ID NO: 12 | SEQ ID NO: 14 |
| 13 | SEQ ID NO: 12 | SEQ ID NO: 15 |
| 14 | SEQ ID NO: 12 | SEQ ID NO: 16 |
| 15 | SEQ ID NO: 21 | SEQ ID NO: 13 |
| 16 | SEQ ID NO: 21 | SEQ ID NO: 14 |
| 17 | SEQ ID NO: 21 | SEQ ID NO: 15 |
| 18 | SEQ ID NO: 21 | SEQ ID NO: 16 |

In some embodiments, the antibody comprises a VH domain comprising or consisting of the sequence of SEQ ID NO: 21, and a VL domain comprising or consisting of the sequence of SEQ ID NO: 13.

In some embodiments of an anti-ROR1 antibody or antigen-binding fragment according to the present disclosure, the antibody or antigen-binding fragment comprises an Fc region, which may be a native or a variant Fc region. In particular embodiments, the Fc region is a human Fc region from IgG1, IgG2, IgG3, IgG4, IgA, IgM, IgE, or IgD. Depending on the utility of the antibody, it may be desirable to use a variant Fc region to change (for example, reduce or eliminate) at least one effector function, for example, ADCC and/or CDC. In some embodiments, the present disclosure provides an anti-ROR1 antibody or antigen-binding fragment comprising an Fc region with one or more mutation to change at least one effector function, for example, L234A and L235A.

In some embodiments, antigen-binding fragments of an anti-ROR1 antibody according to the present disclosure may be for example, Fv, Fab, Fab′, Fab′-SH, F(ab′)2; diabodies; linear antibodies; or single-chain antibody molecules (e.g. scFv).

In one embodiment, an anti-ROR1 antibody described herein or an antigen-binding fragment thereof binds to the ROR1 extracellular domain or a portion thereof. In some embodiments, the ROR1 extracellular domain comprises the amino acid sequence Q30-Y406 of the human ROR1 protein under UniProt Identifier Q01973-1, or the amino acid sequence of SEQ ID NO: 41, or a sequence having at least 80%, 85%, 90%, 91%, 92%, 93%, 94%, 95%, 96%, 97%, 98%, 99% or more identity therewith.

In one embodiment, an anti-ROR1 antibody described herein or an antigen-binding fragment thereof binds to ROR1 at the C-terminus of the ROR1 Ig-like domain. In one embodiment, the antibody binds to ROR1 at the same epitope as an antibody with a VH/VL sequence pair of SEQ ID NOs: 8 and 9 (e.g. ROR1-mAb004). In one embodiment, the antibody competes with an antibody with a VH/VL sequence pair of SEQ ID NOs: 42 and 43 (for example, D10 antibody of WO2012097313) for binding to ROR1.

In an embodiment, an anti-ROR1 antibody described herein or an antigen-binding fragment thereof has an on-rate constant (kₒₙ) to human ROR1 of at least 1×10⁴ M⁻¹ s⁻¹, at least 3×10⁴ M⁻¹ s⁻¹, at least 5×10⁴ M⁻¹ s⁻¹, at least 7×10⁴ M⁻¹ s⁻¹, at least 9×10⁴ M⁻¹ s⁻¹, at least 1×10⁵ M⁻¹ s⁻¹, as measured by biolayer interferometry or surface plasmon resonance.

In another embodiment, an anti-ROR1 antibody described herein or an antigen-binding fragment thereof has an off-rate constant (k_{off}) to human ROR1 of less than 5×10⁻³ s⁻¹, less than 3×10⁻³s⁻¹, less than 2×10⁻³s⁻¹, less than 1×10⁻³ s⁻¹, as measured by surface plasmon resonance or biolayer interferometry. In a further embodiment, an anti-ROR1 antibody described herein or an antigen-binding fragment thereof is a humanized antibody, and has a k_{off} for human ROR1 that is about 1-100%, for example about 3-50% of the k_{off} value for human ROR1 of an antibody with a VH/VL sequence pair of SEQ ID NOs: 8 and 9 in the same antibody format. The off-rate may be used to characterize the binding duration of an antibody to its antigen. In general, a long off-rate correlates with a slow dissociation of the formed complex whereas a short off-rate correlates with a quick dissociation. In one embodiment, the anti-ROR1 antibody described herein, or antigen-binding fragment thereof, has an off-rate slower than that observed for D10 as described in WO2012097313, and stays bound to the target ROR1 longer, which may favor enhanced recruitment of effector molecules to ROR1-expressing (“ROR1⁺”) tumor cells.

In one embodiment, an anti-ROR1 antibody described herein or an antigen-binding fragment thereof has a dissociation constant (K_{D}) to ROR1 in the nanomolar (10⁻⁷ to 10⁻⁹) range, for example, less than 8×10⁻⁷ M, less than 5×10⁻⁷ M, less than 3×10⁻⁷ M, less than 1×10⁻⁷ M, less than 8×10⁻⁸ M, less than 5×10⁻⁸ M, less than 3×10⁻⁸ M, less than 2×10⁻⁸ M, less than 1×10⁻⁸ M, less than 8×10⁻⁸ M, less than 6×10⁻⁹ M, less than 4×10⁻⁹ M, less than 2×10⁻⁹ M, or less than 1×10⁻⁹ M.

In one embodiment, an anti-ROR1 antibody described herein or an antigen-binding fragment thereof specifically binds to ROR1 displayed on ROR1⁺ target cells, such as CHO cell lines or myeloma cell lines expressing ROR1. As measured by flow cytometry in a cell-based assay, the anti-ROR1 antibody displays strong binding potency to ROR1⁺ cells stronger than that observed for D10 as described in WO2012097313. In some embodiments, the cell binding potency is reflected by MFI detected at saturation concentration of antibody or at about 100 nM of antibody concentration. In some embodiments, the anti-ROR1 antibody or antigen-binding fragment described herein displays higher binding potency to ROR1 displayed on the target cell, as compared to an antibody with a VH/VL sequence pair of SEQ ID NOs: 44 and 45 (such as antibody R12 of WO 2014167022), or an antibody binding to the same epitope as R12 at the junction of the Ig and Fz domains of ROR1. In one embodiment, the binding potency of an antibody to ROR1-expressing cells is measured in a cell-based assay as described in Example 1.3.

In some embodiments, as expected, the anti-ROR1 antibody described herein with relatively low affinity for ROR1 in nanomolar range but strong cell surface binding potency could favor distribution into the tumor, and/or lead to a more selective targeting of tumor cells expressing higher densities of the target.

In one embodiment, an anti-ROR1 antibody described herein or an antigen-binding fragment thereof exhibits minimum internalization upon binding to cell surface of ROR1-expressing cells. In one embodiment, the internalization rate is not more than 20%, 15%, 14%, 13%, 12%, 11%, or 10%, or the antibody is not internalized, as measured in a cell-based assay. The internalization rate can be reflected by a decrease percentage in the mean fluorescence intensity (MFI), as detected by flow cytometry, of the antibody binding to the surface of ROR1-expressing cells after a two-hour incubation at 37° C., relative to a control kept at 4° C. for the same period. In one embodiment, the internlization of anti-ROR1 antibody is characterized using ROR1-expressing myeloma cell line. In one embodiment, before MFI is detected, incubation of the test antibody with ROR1-expressing cells is performed for a period, for example at 4° C. for 30 minutes, to allow the antibody binding to ROR1 on the cell surface of the cells, and then the cells are incubated at 37° C. for 2 hours to allow internalization, or kept at 4° C. for the same period to serve as a control. In one embodiment, the internalization rate is calibrated relative to the internalization rate measured in an 37° C. incubation in the presence of an internalization inhibitor such as phenylarsine oxide (PAO). In one embodiment, the degree of internalization is measured in a cell-based assay as described in Example 8.

In one embodiment, the antibody can block ROR1 from binding to its ligand Wnt5a on the cell surface of ROR-expressing cells. In another embodiment, the antibody can be used for inhibiting ROR1/wnt5 signaling. In a further embodiment, the antibody can be used for inhibiting cancer growth and metastasis associated with ROR1/wnt5A pathway.

### Anti-CD3 Antibodies

The present disclosure also provides antibodies capable of binding human CD3.

In some embodiments, an anti-CD3 antibody according to the present disclosure comprises: a set of six CDRs, CDR-H1, CDR-H2, CDR-H3, CDR-L1, CDR-L2, and CDR-L3, wherein:
CDR-H1 comprises the sequence of NYYVH (SEQ ID NO:25);
CDR-H2 comprises the sequence of WISPGSDNTKYNEKFKG (SEQ ID NO: 26);
CDR-H3 comprises the sequence of DDYGNYYFDY (SEQ ID NO: 27);
CDR-L1 comprises the sequence of KSSQSLLNARTRKNYLA (SEQ ID NO: 28);
CDR-L2 comprises the sequence of WASTRES (SEQ ID NO: 29);
CDR-L3 comprises the sequence of KQSYILRT (SEQ ID NO: 30),
wherein the CDRs are defined according to Kabat numbering.

In some embodiments, the anti-CD3 antibody or antigen-binding fragment thereof according to the present application comprises:
a VH domain comprising the sequence of SEQ ID NO: 22 or 23, or a sequence having at least 80%-90%, or 95%-99% identity therewith, and/or
a VL domain comprising the sequence of SEQ ID NO: 24, or a sequence having at least 80%-90%, or 95%-99% identity therewith.

In some embodiments, the anti-CD3 antibody or antigen-binding fragment thereof comprises a VH domain comprising the sequence of SEQ ID NO: 22 and a VL domain comprising the sequence of SEQ ID NO: 24. In other embodiments, the anti-CD3 antibody or antigen-binding fragment thereof comprises a VH domain comprising the sequence of SEQ ID NO: 23 and a VL domain comprising the sequence of SEQ ID NO: 24.

In some embodiments, an anti-ROR1 antibody according to the present disclosure or an anti-CD3 antibody according to the present disclosure may be used to make derivative binding proteins recognizing the same target antigen by techniques well established in the field. Such a derivative may be, e.g., a single-chain antibody (scFv), a Fab fragment (Fab), a Fab′ fragment, an F(ab′)₂, an Fv, and a disulfide linked Fv. Such a derivative may be, e.g., a fusion protein or conjugate comprising the anti-ROR1 antibody according to the present disclosure or an anti-CD3 antibody according to the present disclosure. The fusion protein may be a multi-specific antibody or a CAR molecule. The conjugate may be an antibody-drug conjugate (ADC), or an antibody conjugated with a detection agent such as a radioisotope.

### ROR1×CD3 Bispecific Binding Proteins

In another aspect, the present disclosure provides ROR1/CD3 bispecific binding proteins, especially Fabs-in-Tandem immunoglobulins (FIT-Ig) and Monovalent Asymmetric Tandem Fab bispecific antibodies (MAT-Fab), that are capable of binding to both ROR1 and CD3. Each variable domain (VH or VL) in a FIT-Ig or a MAT-Fab may be obtained from one or more “parental” monoclonal antibodies that bind one of the target antigens, i.e., ROR1 or CD3. FIT-Ig or MAT-Fab binding proteins may be produced using variable domain sequences of anti-ROR1 and anti-CD3 monoclonal antibodies as disclosed herein. For instance, the parental antibodies are humanized antibodies.

An aspect of the present disclosure pertains to selecting parental antibodies with at least one or more properties desired in the FIT-Ig or the MAT-Fab molecule. In an embodiment, the antibody properties are selected from the group consisting of antigen specificity, affinity to antigen, dissociation rate, cell binding potency, internalization rate, biological function, epitope recognition, stability, solubility, production efficiency, immunogenicity, pharmacokinetics, bioavailability, tissue cross reactivity, and orthologous antigen binding.

In some embodiments, bispecific FIT-Ig and MAT-Fab proteins according to the present disclosure are configured without any interdomain peptide linker. Whereas in multivalent engineered immunoglobulin formats having tandem binding sites, it was commonly understood in the field that the adjacent binding sites would interfere with each other unless a flexible linker was used to separate the binding sites spatially. It has been discovered for the ROR1/CD3 FIT-Ig and MAT-Fab of the present disclosure, however, that the arrangement of the immunoglobulin domains according to the chain formulas disclosed herein results in polypeptide chains that are well-expressed in transfected mammalian cells, assemble appropriately, and are secreted as bispecific, multivalent immunoglobulin-like binding proteins that bind the target antigens ROR1 and CD3. See, Examples, infra. Moreover, omission of synthetic linker sequences from the binding proteins can avoid the creation of antigenic sites recognizable by mammalian immune systems, and in this way the elimination of linkers decreases possible immunogenicity of the FIT-Igs and MAT-Fab and leads to a half-life in circulation that is like a natural antibody, that is, the FIT-Ig and MAT-Fab are not rapidly cleared through immune opsonization and capture in the liver.

In some embodiments, an ROR1×CD3 bispecific binding protein according to the present application comprises:
a) a first antigen-binding site that specifically binds ROR1; and
b) a second antigen-binding site that specifically binds CD3.

In one embodiment, the bispecific binding proteins as described herein comprise a set of six CDRs, CDR-H1, CDR-H2, CDR-H3, CDR-L1, CDR-L2, and CDR-L3 derived from any anti-ROR1 antibody or antigen-binding fragment thereof according to the present application and described herein to form the ROR1 binding site of the bispecific binding protein. In some further embodiments, the bispecific binding proteins as described herein comprise a VH/VL pair derived from any anti-ROR1 antibody or antigen-binding fragment thereof according to the present application and described herein to form the ROR1 binding site of the bispecific binding protein.

In one embodiment, the bispecific binding proteins as described herein further comprise a set of six CDRs, CDR-H1, CDR-H2, CDR-H3, CDR-L1, CDR-L2, and CDR-L3 derived from any anti-CD3 antibody or antigen-binding fragment thereof according to the present application and described herein to form the CD3 binding site of the bispecific binding protein. In some further embodiments, the bispecific binding proteins as described herein comprise a VH/VL pair derived from any anti-CD3 antibody or antigen-binding fragment thereof according to the present application and described herein to form the CD3 binding site of the bispecific binding protein.

In one embodiment, the ROR1 binding site and the CD3 binding site in a bispecific ROR1/CD3 binding protein according to the present application are humanized, comprising humanized VH/VL sequences, respectively.

### Bispecific FIT-Ig Binding Proteins

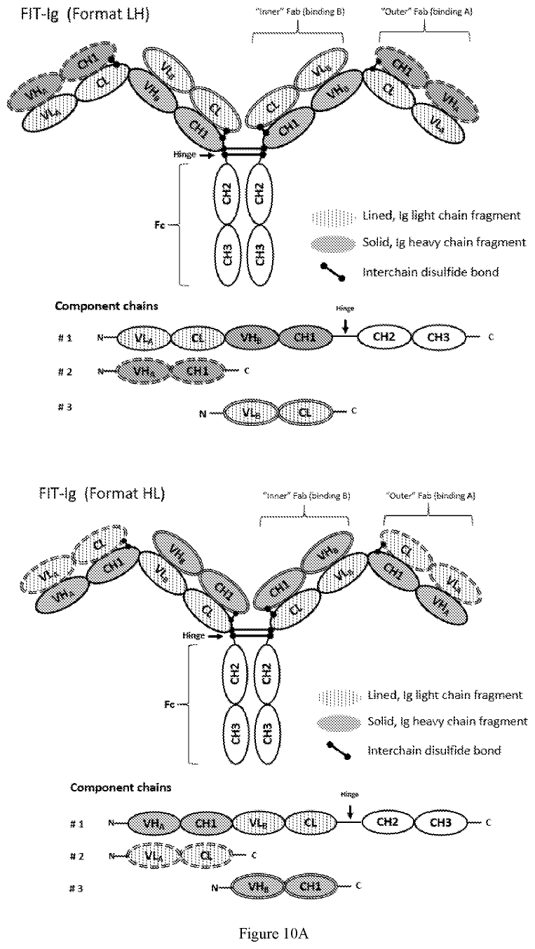

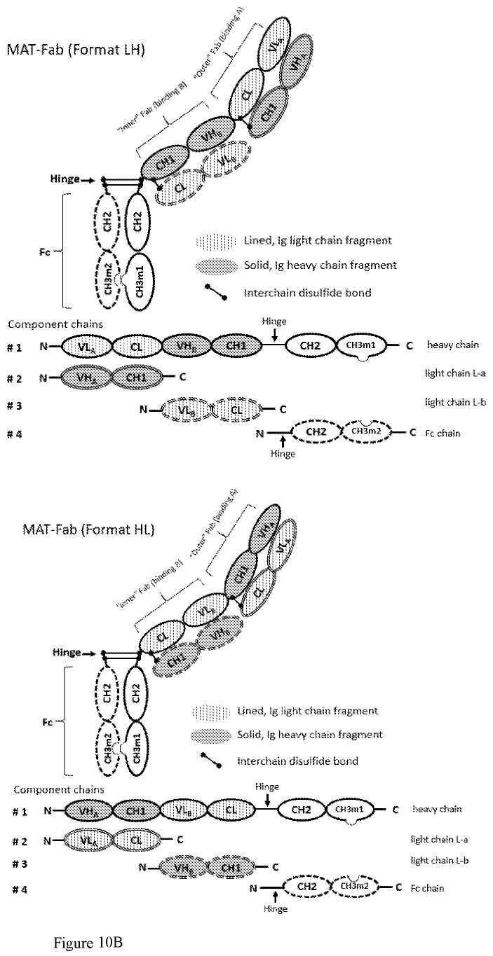

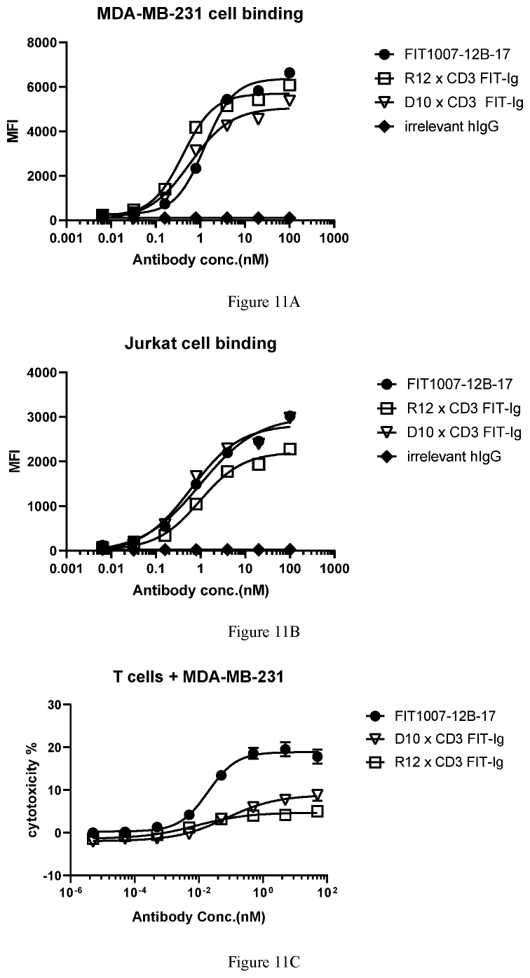

In one embodiment, an ROR1×CD3 bispecific binding protein according to the present application is a bispecific FIT-Ig binding protein capable of binding ROR1 and CD3. A Fabs-in-Tandem immunoglobulin (FIT-Ig) binding protein is a monomeric, dual-specific, tetravalent binding protein comprising six polypeptide chains, and having four functional Fab binding regions with two outer Fab binding regions and two inner Fab binding regions. As shown in FIG. 10A, the binding protein adopts the format (outer Fab-inner Fab-Fc)×2, and binds both antigen A and antigen B. In one aspect, the ROR1×CD3 bispecific binding protein according to the present application is a bispecific FIT-Ig binding protein, wherein two Fab domains of the FIT-Ig protein form the first antigen-binding site that specifically binds ROR1; and the other two Fab domains of the FIT-Ig protein form the second antigen-binding site that specifically binds CD3. In some embodiments, a FIT-Ig binding protein according to the present disclosure employs no linker between immunoglobulin domains.

In a further embodiment, the present disclosure provides a bispecific Fabs-in-Tandem immunoglobulin (FIT-Ig) binding protein comprises a first polypeptide chain, a second polypeptide chain and a third polypeptide chain, wherein
(i) in Format LH, the first polypeptide chain comprises, from amino terminus to carboxyl terminus, VL_{A}-CL-VH_{B}-CH1-Fc wherein CL is fused directly to VH_{B}; the second polypeptide chain comprises, from amino to carboxyl terminus, VH_{A}-CH1; the third polypeptide chain comprises, from amino to carboxyl terminus, VL_{B}-CL; or
(ii) in Format HL, the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1;
wherein VL is a light chain variable domain, CL is a light chain constant domain, VH is a heavy chain variable domain, CH1 is a heavy chain constant domain, Fc is an immunoglobulin Fc region, for example, the Fc of IgG1 (for instance, the Fc comprising, from amino terminus to carboxyl terminus, hinge-CH2-CH3),
wherein VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A is ROR1 and the second antigen B is CD3, or wherein the first antigen A is CD3 and the second antigen B is ROR1,
wherein two of the first polypeptide chains, two of the second polypeptide chains, and two of the third polypeptide chains are associated to form a FIT-Ig binding protein.

In some embodiments of the bispecific FIT-Ig binding protein according the present application, the first polypeptide chain comprises, from amino terminus to carboxyl terminus, VL_{A}-CL-VH_{B}-CH1-Fc, wherein antigen A is ROR1 and antigen B is CD3, or antigen A is CD3 and antigen B is ROR1.

In some embodiments, the Fab binding to ROR1 formed by VL-CL pairing with VH-CH1 in the FIT-Ig binding protein (for example, when A is ROR1, formed by VL_{A}-CL and VH_{A}-CH1; or when B is ROR1, formed by VL_{B}-CL and VH_{B}-CH1) comprises a set of six CDRs, namely CDR-H1, CDR-H2, CDR-H3, CDR-L1, CDR-L2, and CDR-L3, derived from any anti-ROR1 antibody or antigen-binding fragment thereof according to the present application and described herein to form the ROR1 binding site of the bispecific binding protein. In some further embodiments, the CDR-H1, CDR-H2, CDR-H3, CDR-L1, CDR-L2, and CDR-L3 comprise respectively the sequences of SEQ ID NOs: 1, 2, 3 and 5, 6, 7; or the sequences of SEQ ID NOs: 1, 4, 3 and 5, 6, 7.

In some embodiments, the Fab binding to ROR1 in the FIT-Ig binding protein comprises a VH/VL pair derived from any anti-ROR1 antibody or antigen-binding fragment thereof according to the present application and described herein. In some further embodiments, the VH/VL pair comprises the sequences selected from the group consisting of the following VH/VL sequence pairs: SEQ ID NOs: 8/9, 17/9, 10/13, 10/14, 10/15, 10/16, 11/13, 11/14, 11/15, 11/16, 12/13, 12/14, 12/15, 12/16, and 21/13, or sequences having at least 80%, 85%, 90%, 95% or 99% identity therewith. In some embodiments, the Fab binding to ROR1 in the FIT-Ig binding protein comprises a VH sequence of SEQ ID NO: 21 and a VL sequence of SEQ ID NO: 13.

In some embodiments, the Fab binding to CD3 formed by VL-CL pairing with VH-CH1 in the FIT-Ig binding protein (for example, when A is CD3, formed by VL_{A}-CL and VH_{A}-CH1; or when B is CD3, formed by VL_{B}-CL and VH_{B}-CH1) comprises a set of six CDRs, namely CDR-H1, CDR-H2, CDR-H3, CDR-L1, CDR-L2, and CDR-L3, derived from any anti-CD3 antibody or antigen-binding fragment thereof according to the present application and described herein to form the CD3 binding site of the bispecific binding protein. In some embodiments, the Fab binding to CD3 formed by VL-CL pairing with VH-CH1 in the FIT-Ig binding protein comprises a set of six CDRs, wherein CDR-H1, CDR-H2, CDR-H3, CDR-L1, CDR-L2, and CDR-L3 comprise the sequences of SEQ ID NOs: 25, 26, 27 and 28, 29, 30 respectively. In some further embodiments, the Fab binding to CD3 comprises a VH/VL pair comprising the sequences of SEQ ID NOs: 22 and 24, or sequences having at least 80%, 85%, 90%, 95% or 99% identity therewith; or the sequences of SEQ ID NOs: 23 and 24, or sequences having at least 80%, 85%, 90%, 95% or 99% identity therewith.

In a further embodiment, the present disclosure provides a bispecific Fabs-in-Tandem immunoglobulin (FIT-Ig) binding protein comprising first, second, and third polypeptide chains,
wherein
(i) in Format LH, the first polypeptide chain comprises, from amino to carboxyl terminus, VL_{A}-CL-VH_{B}-CH1-Fc wherein CL is directly fused to VH_{B}; the second polypeptide chain comprises, from amino to carboxyl terminus, VH_{A}-CH1; the third polypeptide chain comprises, from amino to carboxyl terminus, VL_{B}-CL; or
(ii) in Format HL, the first polypeptide chain comprises, from amino to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1;
wherein VL is a light chain variable domain, CL is a light chain constant domain, VH is a heavy chain variable domain, CH1 is a heavy chain constant domain, Fc is an immunoglobulin Fc region, A is an epitope of ROR1 and B is an epitope of CD3, or A is an epitope of CD3 and B is an epitope of ROR1. In accordance with the present disclosure, such FIT-Ig binding proteins bind to both ROR1 and CD3.

In some embodiments, the Fab fragments of such FIT-Ig binding proteins incorporate VL_{A}-CL and VH_{A}-CH1 domains from a parental antibody binding to one of the antigens ROR1 and CD3, and incorporate VL_{B}-CL and VH_{B}-CH1 domains from a different parental antibody binding to the other of the antigens ROR1 and CD3. In some embodiments, VH-CH1::VL-CL pairing results in tandem Fab moieties recognizing both ROR1 and CD3.

In accordance with the present disclosure, an ROR1/CD3 FIT-Ig binding protein comprises first, second, and third polypeptide chains, wherein the first polypeptide chain comprises, from amino to carboxyl terminus, VL_{ROR1}-CL-VH_{CD3}-CH1-hinge-CH2-CH3 wherein CL is directly fused to VH_{CD3}, wherein the second polypeptide chain comprises, from amino to carboxyl terminus, VH_{ROR1}-CH1; and wherein the third polypeptide chain comprises, from amino to carboxyl terminus, VL_{CD3}-CL. In alternative embodiments, an ROR1/CD3 FIT-Ig binding protein comprises first, second, and third polypeptide chains, wherein the first polypeptide chain comprises, from amino to carboxyl terminus, VH_{ROR1}-CH1-VL_{CD3}-CL-hinge-CH2-CH3 wherein CH1 is directly fused to VL_{CD3}, wherein the second polypeptide chain comprises, from amino to carboxyl terminus, VL_{ROR1}-CL; and wherein the third polypeptide chain comprises, from amino to carboxyl terminus, VH_{CD3}-CH1. In some embodiments, VL_{ROR1} is a light chain variable domain of an anti-ROR1 antibody, CL is a light chain constant domain, VH_{ROR1} is a heavy chain variable domain of an anti-ROR1 antibody, CH1 is a heavy chain constant domain, VL_{CD3} is a light chain variable domain of an anti-CD3 antibody, VH_{CD3} is a heavy chain variable domain of an anti-CD3 antibody; and optionally, the domains VL_{CD3}-CL are the same as the light chain of an anti-CD3 parental antibody, the domains VH_{CD3}-CH1 are the same as the heavy chain variable and heavy chain constant domains of an anti-CD3 parental antibody, the domains VL_{ROR1}-CL are the same as the light chain of an anti-ROR1 parental antibody, and the domains VH_{ROR1}-CH1 are the same as the heavy chain variable and heavy chain constant domains of an anti-ROR1 parental antibody.

In the foregoing formulas for a FIT-Ig binding protein, an Fc region may be a native or a variant Fc region. In particular embodiments, the Fc region is a human Fc region from IgG1, IgG2, IgG3, IgG4, IgA, IgM, IgE, or IgD. In particular embodiments, the Fc is a human Fc from IgG1, or a modified human Fc comprising one or more mutations to reduce or eliminate at least one Fc effector function, for example the binding of the Fc to FcγR, ADCC and/or CDC. The mutations may be for example, L234A/L235A (numbering according to Kabat EU index). In one embodiment, the Fc region is of human IgG1 with the mutations L234A and L235A, such as set forth in Table 8, infra (aa104 to aa 227 of SEQ ID NO:31). In one embodiment, the Fc region comprises the sequence of aa104 to aa 227 of SEQ ID NO:31, or a sequence having at least 90%, 95%, 97%, 98%, 99% or more identity herewith.

In some embodiments of a FIT-Ig binding protein according to the present disclosure, CH1, CL and Fc domains are of or from human sequences. In some embodiments of a FIT-Ig binding protein according to the present disclosure, CH1 is a human IgG1 constant CH1 domain, for example, having the sequence of SEQ ID NO: 33, or a sequence having at least 90%, 95%, 97%, 98%, 99% or more identity herewith. In the foregoing formulas for a FIT-Ig binding protein, CL is a human constant kappa CL domain, for instance, having the sequence of SEQ ID NO: 32, or a sequence having at least 90%, 95%, 97%, 98%, 99% or more identity herewith.

In an embodiment, FIT-Ig binding proteins of the present disclosure retain one or more properties of the parental antibodies. In some embodiments, the FIT-Ig retains binding affinity for the target antigens (i.e., CD3 and ROR1) comparable to that of the parental antibodies, meaning that the binding affinity of the FIT-Ig binding protein for the ROR1 and CD3 antigen targets does not vary by greater than in comparison to the binding affinity of the parental antibodies for their respective target antigens, as measured by surface plasmon resonance or biolayer interferometry.

In one embodiment, a FIT-Ig binding protein of the present disclosure binds ROR1 and CD3, and is comprised of a first polypeptide chain, a second polypeptide chain, and a third polypeptide chain, wherein:
the first polypeptide chain comprises an amino acid sequence of SEQ ID NO:34 or 37, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith.

In one embodiment, a FIT-Ig binding protein of the present disclosure binds ROR1 and CD3, and is comprised of a first polypeptide chain comprising, consisting essentially of, or consisting of the sequence of SEQ ID NO:34 or 37; a second polypeptide chain comprising, consisting essentially of, or consisting of the sequence of SEQ ID NO:35; and a third polypeptide chain comprising, consisting essentially of, or consisting of the sequence of SEQ ID NO:36.

### Bispecific MAT-Fab Binding Protein

In one embodiment, an ROR1×CD3 bispecific binding protein according to the present application is a bispecific MAT-Fab binding protein capable of binding ROR1 and CD3. A Monovalent Asymmetric Tandem Fab (MAT-Fab) bispecific binding protein is a monomeric, dual-specific, bi-valent binding protein comprising four polypeptide chains and having two functional Fab binding regions in tandem. As shown in FIG. 10B, the binding protein adopts the outer Fab-inner Fab-Fc:Fc dimer format, and binds both antigen A and antigen B. In some embodiments, the ROR1×CD3 bispecific binding protein according to the present application is a bispecific MAT-Fab binding protein, wherein one Fab domain of the MAT-Fab protein forms the first antigen-binding site that specifically binds ROR1; and the other Fab domain of the MAT-Fab protein forms the second antigen-binding site that specifically binds CD3.

In a further embodiment, the present disclosure provides a bispecific monovalent asymmetric tandem Fab (MAT-Fab) binding protein comprising a first polypeptide chain, a second polypeptide chain, a third polypeptide chain and a fourth polypeptide chain, wherein
(i) in Format LH, the first polypeptide chain comprises, from amino terminus to carboxyl terminus, VL_{A}-CL-VH_{B}-CH1-Fc wherein CL is fused directly to VH_{B}; the second polypeptide chain comprises, from amino to carboxyl terminus, VH_{A}-CH1; the third polypeptide chain comprises, from amino to carboxyl terminus, VL_{B}-CL; and the fourth polypeptide chain comprises a Fc; or
(ii) in Format HL, the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1; and the fourth polypeptide chain comprises a Fc;
wherein VL is a light chain variable domain, CL is a light chain constant domain, VH is a heavy chain variable domain, CH1 is a heavy chain constant domain, Fc is an immunoglobulin Fc region, for example, the Fc of IgG1 (for instance, the Fc comprising, from amino terminus to carboxyl terminus, hinge-CH2-CH3),
wherein VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A is ROR1, and the second antigen B is CD3, or wherein the first antigen A is CD3, and the second antigen B is ROR1,
wherein the first polypeptide chain, the second polypeptide chain, the third polypeptide chain and the fourth polypeptide chain are associated to form a MAT-Fab binding protein.

In some embodiments of the MAT-Fab binding protein according to the present disclosure, the Fc is an immunoglobulin Fc region comprising, from amino terminus to carboxyl terminus, hinge-CH2-CH3, wherein hinge-CH2 is the hinge-CH2 region of an immunoglobulin heavy chain and wherein the hinge-CH2 is fused directly to CH3, and wherein the Fc region of the first polypeptide chain comprises a first CH3 domain (a CH3m1 domain), and the Fc region of the fourth polypeptide chain comprise a second CH3 domain (a CH3m2 domain). In further embodiments, the Fc regions of the first and the fourth polypeptide chains, especially in their CH3 domains, comprise heterodimerizing modifications, which favor heterodimerization over homodimerization of the two Fc regions. In some embodiments, knob-into-hole heterodimerization technology is used to favor the heterodimerization of the chains. Optionally, the MAT-Fab binding protein further comprises a mutation in the first CH3 domain (CH3m1 domain) and the second CH3 domain (CH3m2 domain) to introduce a cysteine residue to favor disulfide bond formation in pairing the two CH3 domains.

In some embodiments, one or more knob-into-hole (KiH) mutations are introduced into the first CH3 domain (CH3m1 domain) of the first chain and the second CH3 domain (CH3m2 domain) of the fourth chain. In a further embodiment, when the first CH3 domain (CH3m1 domain) of the first chain has been mutated to form a structural knob, then the second CH3 domain (CH3m2 domain) of the fourth chain has been mutated to form a complementary structural hole to favor pairing of the first CH3 domain with the second CH3 domain; or when the first CH3 domain (CH3m1 domain) of the first chain has been mutated to form a structural hole, then the second CH3 domain (CH3m2 domain) of the fourth chain has been mutated to form a complementary structural knob to favor pairing of the first CH3 domain with the second CH3 domain. In some embodiments, the “knob” mutation is a T366W substitution, and the complementary “hole” mutations are T366S, L368A and Y407V substitutions.

In some embodiments, the bispecific binding protein according to the present disclosure is a MAT-Fab protein with a typical knob (T366W) substitution in the first CH3 domain and the corresponding hole substitutions (T366S, L368A and Y407V) in the second CH3 domain, and optionally with two additional introduced cysteine residues S354C/Y349C (contained in the respective corresponding CH3 sequences). For example, the first CH3 domain (CH3m1 domain) may comprise a knob substitution T366W and an introduced cysteine residue S354C, and the second CH3 domain (CH3m2 domain) comprises T366S, L368A and Y407V as hole substitutions and an introduced cysteine residue Y349C.

The knobs-into-holes dimerization modules and their use in antibody engineering are well-known in the art and described, e.g., in Ridgway et al., 1996, Protein Engineering 9(7) 617-621. The introducing of additional disulfide bridge in the CH3 domain is reported, e.g., in Merchant, A. M., et al., Nat. Biotechnol. 16 (1998) 677-681.

In some embodiments of the bispecific MAT-Fab binding protein according the present application, the first polypeptide chain comprises, from amino terminus to carboxyl terminus, VL_{A}-CL-VH_{B}-CH1-Fc, wherein antigen A is ROR1, antigen B is CD3, or antigen A is CD3, antigen B is ROR1.

In some embodiments, the Fab binding to ROR1 formed by VL-CL pairing with VH-CH1 in the MAT-Fab binding protein (for example, when A is ROR1, formed by VL_{A}-CL and VH_{A}-CH1) comprises a set of six CDRs, namely CDR-H1, CDR-H2, CDR-H3, CDR-L1, CDR-L2, and CDR-L3, derived from any anti-ROR1 antibody or antigen-binding fragment thereof according to the present application and described herein to form the ROR1 binding site of the bispecific binding protein. In some embodiments, the CDR-H1, CDR-H2, CDR-H3, CDR-L1, CDR-L2, and CDR-L3 comprise respectively the sequences of SEQ ID NOs: 1, 2, 3 and 5, 6, 7; or comprise respectively the sequences of SEQ ID NOs: 1, 4, 3 and 5, 6, 7. In some embodiments, the Fab binding to ROR1 in the MAT-Fab binding protein comprises a VH/VL pair derived from any anti-ROR1 antibody or antigen-binding fragment thereof according to the present application and described herein to form the ROR1 binding site of the bispecific binding protein. In some embodiments, the VH/VL pair comprises the sequences selected from the group consisting of the following VH/VL sequence pairs: SEQ ID NOs: 8/9, 17/9, 10/13, 10/14, 10/15, 10/16, 11/13, 11/14, 11/15, 11/16, 12/13, 12/14, 12/15, 12/16, and 21/13, or sequences having at least 80%, 85%, 90%, 95% or 99% identity therewith. In some embodiments, the Fab binding to ROR1 in the MAT-Fab binding protein comprises a VH sequence of SEQ ID NO: 21 and a VL sequence of SEQ ID NO: 13.

In some embodiments, the Fab binding to CD3 formed by VL-CL pairing with VH-CH1 in the MAT-Fab binding protein (for example, when B is CD3, formed by VL_{B}-CL and VH_{B}-CH1) comprises a set of six CDRs, CDR-H1, CDR-H2, CDR-H3, CDR-L1, CDR-L2, and CDR-L3 derived from any anti-CD3 antibody or antigen-binding fragment thereof according to the present application and described herein to form the CD3 binding site of the bispecific binding protein. In some embodiments, the Fab binding to CD3 formed by VL-CL pairing with VH-CH1 in the MAT-Fab binding protein comprises a set of six CDRs, CDR-H1, CDR-H2, CDR-H3, CDR-L1, CDR-L2, and CDR-L3, comprising respectively the sequences of SEQ ID NOs: 25, 26, 27 and 28, 29, 30. In some further embodiments, the Fab binding to CD3 comprise a VH/VL pair comprising the sequences of SEQ ID NOs: 22 and 24, or sequences having at least 80%, 85%, 90%, 95% or 99% identity therewith; or the sequences of SEQ ID NOs: 23 and 24, or sequences having at least 80%, 85%, 90%, 95% or 99% identity therewith.

In a further embodiment, this disclosure provides a bispecific monovalent asymmetric tandem Fab (MAT-Fab) binding protein comprising a first polypeptide chain, a second polypeptide chain, a third polypeptide chain and a fourth polypeptide chain, wherein:
(i) in Format LH, the first polypeptide chain comprises, from amino to carboxyl terminus, VL_{A}-CL-VH_{B}-CH1-Fc wherein CL is directly fused to VH_{B}; the second polypeptide chain comprises, from amino to carboxyl terminus, VH_{A}-CH1; the third polypeptide chain comprises, from amino to carboxyl terminus, VL_{B}-CL, and the fourth polypeptide chain comprises a Fc; or
(ii) in Format HL, the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1, and the fourth polypeptide chain comprises a Fc;
wherein VL is a light chain variable domain, CL is a light chain constant domain, VH is a heavy chain variable domain, CH1 is a heavy chain constant domain, Fc is an immunoglobulin Fc region comprising from amino terminus to carboxyl terminus hinge-CH2-CH3, A is an epitope of ROR1 and B is an epitope of CD3, or A is an epitope of CD3 and B is an epitope of ROR1. In accordance with the present disclosure, such MAT-Fab binding proteins bind to both ROR1 and CD3.

In some embodiments, the Fab fragments of such MAT-Fab binding proteins incorporate VL_{A}-CL and VH_{A}-CH1 domains from a parental antibody binding to one of the antigens ROR1 and CD3 (such as those anti-ROR1 or anti-CD3 describe herein), and incorporate VL_{B}-CL and VH_{B}-CH1 domains from a different parental antibody binding to the other of the antigens ROR1 and CD3 (such as those anti-ROR1 or anti-CD3 describe herein). In some embodiments, VH-CH1::VL-CL pairing results in tandem Fab moieties recognizing both ROR1 and CD3.

In accordance with the present disclosure, an ROR1/CD3 MAT-Fab binding protein comprises first, second, third and fourth polypeptide chains, wherein the first polypeptide chain comprises, from amino to carboxyl terminus, VL_{ROR1}-CL-VH_{CD3}-CH1-hinge-CH2-CH3m1 wherein CL is directly fused to VH_{CD3}; wherein the second polypeptide chain comprises, from amino to carboxyl terminus, VH_{ROR1}-CH1; wherein the third polypeptide chain comprises, from amino to carboxyl terminus, VL_{CD3}-CL; and wherein the fourth polypeptide chain is an Fc polypeptide chain comprising hinge-CH2-CH3m2. In alternative embodiments, an ROR1/CD3 MAT-Fab binding protein comprises first, second, third and fourth polypeptide chains, wherein the first polypeptide chain comprises, from amino to carboxyl terminus, VH_{ROR1}-CH1-VL_{CD3}-CL-hinge-CH2-CH3m1 wherein CH1 is directly fused to VL_{CD3}; wherein the second polypeptide chain comprises, from amino to carboxyl terminus, VL_{ROR1}-CL; wherein the third polypeptide chain comprises, from amino to carboxyl terminus, VH_{CD3}-CH1; and wherein the fourth polypeptide chain is an Fc polypeptide chain comprising hinge-CH2-CH3m2. In some embodiments, VL_{ROR1} is a light chain variable domain of an anti-ROR1 antibody, CL is a light chain constant domain, VH_{ROR1} is a heavy chain variable domain of an anti-ROR1 antibody, CH1 is a heavy chain constant domain, VL_{CD3} is a light chain variable domain of an anti-CD3 antibody, VH_{CD3} is a heavy chain variable domain of an anti-CD3 antibody, and one or more “knobs-in-holes” mutations are introduced into CH3m1 and CH3m2 domains to favor heterodimerization of the CH3m1 and CH3m2 domains; and, the domains VL_{CD3}-CL are the same as the light chain of an anti-CD3 parental antibody, the domains VH_{CD3}-CH1 are the same as the heavy chain variable and heavy chain constant domains of an anti-CD3 parental antibody, the domains VL_{ROR1}-CL are the same as the light chain of an anti-ROR1 parental antibody, and the domains VH_{ROR1}-CH1 are the same as the heavy chain variable and heavy chain constant domains of an anti-ROR1 parental antibody.

In the foregoing formulas for the first polypeptide chain of a MAT-Fab binding protein, an Fc region may be a native or a variant Fc region. In particular embodiments, the Fc region is a human Fc region from IgG1, IgG2, IgG3, IgG4, IgA, IgM, IgE, or IgD. In particular embodiments, the Fc is a human Fc from IgG, or variant thereof. In some embodiment, the Fc region is a variant Fc region comprising mutations to reduce or eliminate at least one effector function of the Fc region, for example, the binding of the Fc to FcγR, ADCC and/or CDC. The mutations may be for example, L234A and L235A (numbering according to Kabat EU index). In one embodiment, the Fc region is of human IgG1 with the mutations L234A and L235A.

In some embodiments of a MAT-Fab binding protein according to the present disclosure, CH1, CL and Fc domains are of or from human sequences. In some embodiments of a MAT-Fab binding protein according to the present disclosure, CH1 is a heavy chain constant domain, for instance, a human IgG1 constant CH1 domain, e.g., having the sequence of SEQ ID NO: 33, or a sequence having at least 90%, 95%, 97%, 98%, 99% or more identity herewith. In the foregoing formulas for a MAT-Fab binding protein, CL is a light chain constant domain, for instance, a human constant kappa CL domain, e.g., having the sequence of SEQ ID NO: 32, or a sequence having at least 90%, 95%, 97%, 98%, 99% or more identity herewith.

In some embodiments, a MAT-Fab binding protein according to the present disclosure employs no linker between the immunoglobulin domains.

In an embodiment, MAT-Fab binding proteins of the present disclosure retain one or more properties of the parental antibodies. In some embodiments, the MAT-Fab retains binding affinity for the target antigens (i.e., CD3 and ROR1) comparable to that of the parental antibodies, meaning that the binding affinity of the MAT-Fab binding protein for the ROR1 and CD3 antigen targets does not vary by greater than 10-fold in comparison to the binding affinity of the parental antibodies for their respective target antigens, as measured by surface plasmon resonance or biolayer interferometry.

In one embodiment, a MAT-Fab binding protein of the present disclosure binds ROR1 and CD3 and is comprised of a first polypeptide chain, a second polypeptide chain, and a third polypeptide chain and a fourth polypeptide, wherein:
the first polypeptide chain comprises an amino acid sequence of SEQ ID NO:38 or 40, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith,
the third polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith; and
the fourth polypeptide chain comprises an amino acid sequence of SEQ ID NO:39, or a sequence having at least 80%, 85%, 90%, 91%, 92%, 93%, 94%, 95%, 96%, 97%, 98%, 99% or more identity therewith.

In one embodiment, a MAT-Fab binding protein of the present disclosure binds ROR1 and CD3 and is comprised of a first polypeptide chain comprising, consisting essentially of, or consisting of the sequence of SEQ ID NO:38 or 40; a second polypeptide chain comprising, consisting essentially of, or consisting of the sequence of SEQ ID NO:35; a third polypeptide chain comprising, consisting essentially of, or consisting of the sequence of SEQ ID NO:36; and a fourth polypeptide chain comprises an amino acid sequence of SEQ ID NO:39.

### Properties of Bispecific Binding Proteins

In one embodiment, a bispecific ROR1/CD3 FIT-Ig or MAT-Fab binding protein capable of binding both CD3 and ROR1 as described herein comprises a humanized ROR binding site, or a chimeric ROR1 binding site, for instance, a humanized ROR binding site. In one embodiment, the humanized ROR1 binding site in the FIT-Ig or MAT-Fab protein format has a slower off-rate for ROR1 binding, relative to the chimeric ROR1 binding site in the same FIT-Ig or MAT-Fab format, which consists of VH and VL pair of SEQ ID NOs: 8 and 9. In a further embodiment, the off-rate ratio of the humanized ROR1 binding site relative to the chimeric ROR1 binding site is less than 90%, 80%, 70%, 60%, 50%, 40%, 30%, 20%, 15%, 10%, 5%, as measured by surface plasmon resonance or biolayer interferometry. In one embodiment, the off-rate of a FIT-Ig binding protein described herein for ROR1 is less than 2×10⁻³ s⁻¹, 1×10⁻³ s⁻¹, 8×10⁻⁴s⁻¹, 6×10⁻⁴ s⁻¹, 5×10⁻⁴ s⁻¹, 4×10⁻⁴ s⁻¹, 3×10⁻⁴ s⁻¹, 2×10⁻⁴ s⁻¹, 1×10⁻⁴ s⁻¹, 8×10⁻⁵ s⁻¹, 6×10⁻⁵ s⁻¹, as measured by surface plasmon resonance or biolayer interferometry. In one embodiment, a FIT-Ig binding protein antibody described herein or antigen-binding fragment thereof has a dissociation constant (K_{D}) to ROR1 in the 10⁻⁸ to 10⁻¹⁰ range, for example, less than 8×10⁻⁸ M, less than 5×10⁻⁸ M, less than 3×10⁻⁸ M, less than 2×10⁻⁸ M, less than 1×10⁻⁸ M, less than 8×10⁻⁹ M, less than 6×10⁻⁹ M, less than 4×10⁻⁹ M, less than 2×10⁻⁹ M, or less than 1×10⁻⁹ M, less than 8×10⁻¹⁰ M, less than 6×10⁻¹⁰ M, less than 4×10⁻¹⁰ M, less than 2×10⁻¹⁰ M, or less than 1×10⁻¹⁰ M. In one embodiment, a FIT-Ig binding protein antibody described herein or antigen-binding fragment thereof has an off-rate in the range of 1×10⁻³ s⁻¹ to 1×10⁻⁴s⁻¹, for example, less than 2×10⁻⁴ s⁻¹, and a K_{D} in the range of 1×10⁻⁹ s⁻¹ to 1×10⁻¹⁰ s⁻¹, for example, less than 6×10⁻¹⁰ s⁻¹, in terms of ROR1 binding.

In one embodiment, a bispecific ROR1/CD3 FIT-Ig binding protein or MAT-Fab binding protein capable of binding CD3 and ROR1 as described herein can be expressed in cultures of transfected mammalian host cells such as CHO cells or HEK293 cells at levels greater than 10 mg of ROR1/CD3 FIT-Ig or MAT-Fab binding protein per liter of cell culture (>10 mg/L). In one embodiment, the expression level of the FIT-Ig or MAT-Fab binding protein is greater than 15 mg/L, for example, 15 mg/L to 100 mg/L, or more. In another embodiment, the expression level of FIT-Ig or MAT-Fab binding protein is greater than 20 mg/L.

In one embodiment, a bispecific ROR1/CD3 FIT-Ig binding protein or MAT-Fab binding protein capable of binding CD3 and ROR1 as described herein, after a one-step purification from cell culture media using a Protein A affinity chromatography, have a purity of no less than 90% as detected by SEC-HPLC. In one embodiment, the one-step purified binding proteins have a purity of no less than 91%, 92%, 93%, 95%, 97%, 99% as detected by SEC-HPLC.

In one embodiment, a bispecific ROR1/CD3 FIT-Ig binding protein or MAT-Fab binding protein as described herein exhibits minimum internalization upon binding to cell surface of ROR1-expressing cells, by the cells. In one embodiment, the internalization rate is not more than 20%, 15%, 14%, 13%, 12%, 11%, 10%, or the binding protein is not internalized, according to a cell based assay.

In one embodiment, a bispecific ROR1/CD3 FIT-Ig binding protein or MAT-Fab binding protein as described herein is capable of binding both CD3-expressing cells and ROR1-expressing cells. In one embodiment, the CD3-expressing cells are human TCR/CD3 complex transfected CHO cell lines, or human T cells. In one embodiment, the ROR1-expressing cells are ROR1-expressing tumor cells, for example, human non-small cell lung cancer cells, human breast cancer cells, lung carcinoma cells, or myeloma cells.

In one embodiment, as measured by flow cytometry in a cell-based assay, the binding potency of the bispecific FIT-Ig binding protein to the ROR1-expressing cells are equivalent to or comparable to the corresponding parental anti-ROR1 monoclonal IgG antibody comprising the same VH/VL sequence pairs for ROR1 binding as the bispecific FIT-Ig protein. In one embodiment, the binding potency of the bispecific FIT-Ig binding protein to the CD3-expressing cells are equivalent to, or relatively lower than (but no more than a 10-fold difference, for instance, no more than 2-fold, 1-fold, or 50% decrease) the corresponding parental anti-CD3 monoclonal IgG antibody comprising the same VH/VL sequence pairs for CD3 binding as the bispecific binding protein, as measured by flow cytometry, such as in an assay described in Example 4.

In one embodiment, a bispecific binding protein described herein is capable of modulating a biological function of ROR1, CD3, or both. In one embodiment, the bispecific ROR1/CD3 FIT-Ig binding protein or MAT-Fab binding protein as described herein is capable of activating CD3 signaling in terms of ROR1 dependence. In one embodiment, the bispecific binding proteins of the present disclosure exhibit ROR1-dependent activation of T cells. In one embodiment, a bispecific ROR1/CD3 FIT-Ig binding protein or MAT-Fab binding protein as described herein exhibits ROR1-redirected T cell cytotoxicity. In one embodiment, the bispecific binding proteins of the present disclosure is used for redirecting the cytotoxic activity of T-cells towards ROR1 expressing cells in a non-MHC restricted fashion.

In one embodiment, a bispecific ROR1/CD3 FIT-Ig binding protein or MAT-Fab binding protein as described herein exhibits ROR1-dependent CD3 activation. In one embodiment, upon binding to ROR1-expressing cells, the bispecific ROR1/CD3 antibodies induce the crosslink of CD3/TCR complex on T cells and activation of CD3 signaling. In one embodiment, the ratio of target ROR1-expressing cells to effector T cells is about 1:1. In a further embodiment, the bispecific ROR1/CD3 binding proteins exhibit increased T cell activation in the presence of ROR1-expressing target cells, and much less non-target redirected CD3 activation in the absence of ROR1-expressing target cells, in comparison to corresponding parental anti-CD3 monoclonal IgG antibodies comprising the same VH/VL sequence pairs for CD3 binding as the bispecific FIT-Ig or MAT-Fab proteins, for example as measured at ratio of about 1:1 target cells to effector T cells.

In one embodiment, a bispecific ROR1/CD3 FIT-Ig binding protein or MAT-Fab binding protein as described herein redirect T cell cytotoxicity to ROR1-expressing tumor cells. In another embodiment, a bispecific ROR1/CD3 FIT-Ig binding protein or MAT-Fab binding protein as described herein exhibits anti-tumor activities, such as reducing tumor burden, inhibiting tumor growth, or suppressing neoplastic cell expansion.

### Pharmaceutical Compositions

The present disclosure also provides pharmaceutical compositions comprising an antibody, or antigen-binding portion thereof, or a bispecific multivalent binding protein of the present disclosure (i.e., the primary active ingredient) and a pharmaceutically acceptable carrier. In a specific embodiment, a composition comprises one or more antibodies or binding proteins of the present disclosure. The present disclosure also provides pharmaceutical compositions comprising a combination of anti-ROR1 and anti-CD3 antibodies as described herein, or antigen-binding fragment(s) thereof, and a pharmaceutically acceptable carrier. In particular, the present disclosure provides pharmaceutical compositions comprising at least one FIT-Ig binding protein capable of binding ROR1 and CD3 and a pharmaceutically acceptable carrier. In particular, the present disclosure provides pharmaceutical compositions comprising at least one MAT-Fab binding protein capable of binding ROR1 and CD3 and a pharmaceutically acceptable carrier. Pharmaceutical compositions of the present disclosure may further comprise at least one additional active ingredient. In some embodiments, such an additional ingredient includes, but is not limited to, a prophylactic and/or therapeutic agent, a detection agent, such as an anti-tumor drug, a cytotoxic agent, an antibody of different specificity or functional fragment thereof, a detectable label or reporter. In an embodiment, the pharmaceutical composition comprises one or more additional prophylactic or therapeutic agents, i.e., agents other than the antibodies or binding proteins of the present disclosure, for treating a disorder in which ROR1 activity is detrimental. In an embodiment, the additional prophylactic or therapeutic agents are known to be useful for, have been used, or are currently being used in the prevention, treatment, management, or amelioration of, a disorder or one or more symptoms thereof.

The pharmaceutical compositions comprising proteins of the present disclosure are for use in, but not limited to, diagnosing, detecting, or monitoring a disorder; treating, managing, or ameliorating a disorder or one or more symptoms thereof; and/or research. In some embodiments, the composition may further comprise a carrier, diluent, or excipient. An excipient is generally any compound or combination of compounds that provides a desired feature to a composition other than that of the primary active ingredient (i.e., other than an antibody, functional portion thereof, or binding protein of the present disclosure).

### Nucleic Acid, Vector, and Host Cells

In a further aspect, this disclosure provides isolated nucleic acids encoding one or more amino acid sequences of an anti-ROR1 antibody of this disclosure or an antigen-binding fragment thereof; isolated nucleic acids encoding one or more amino acid sequences of an anti-CD3 antibody of this disclosure or an antigen-binding fragment thereof; and isolated nucleic acids encoding one or more amino acid sequences of a bispecific binding protein, including Fabs-in-Tandem immunoglobulin (FIT-Ig) and MAT-Fab binding protein, capable of binding both ROR1 and CD3. Such nucleic acids may be inserted into a vector for carrying out various genetic analyses or for expressing, characterizing, or improving one or more properties of an antibody or binding protein described herein. A vector may comprise one or more nucleic acid molecules encoding one or more amino acid sequences of an antibody or binding protein described herein in which the one or more nucleic acid molecules is operably linked to appropriate transcriptional and/or translational sequences that permit expression of the antibody or binding protein in a particular host cell carrying the vector. Examples of vectors for cloning or expressing nucleic acids encoding amino acid sequences of binding proteins described herein include, but are not limited to, pcDNA, pTT, pTT3, pEFBOS, pBV, pJV, and pBJ, and derivatives thereof.

The present disclosure also provides a host cell expressing, or capable of expressing, a vector comprising a nucleic acid encoding one or more amino acid sequences of an antibody or binding protein described herein. Host cells useful in the present disclosure may be prokaryotic or eukaryotic. An exemplary prokaryotic host cell is Escherichia coli. Eukaryotic cells useful as host cells in the present disclosure include protist cells, animal cells, plant cells, and fungal cells. An exemplary fungal cell is a yeast cell, including Saccharomyces cerevisiae. An exemplary animal cell useful as a host cell according to the present disclosure includes, but is not limited to, a mammalian cell, an avian cell, and an insect cell. Exemplary mammalian cells include, but are not limited to, CHO cells, HEK cells, and COS cells.

### Methods for Production

In another aspect, the present disclosure provides a method of producing an anti-ROR1 antibody or a functional fragment thereof comprising culturing a host cell comprising an expression vector encoding the antibody or functional fragment in culture medium under conditions sufficient to cause the host cell to express the antibody or fragment capable of binding ROR1.

In another aspect, the present disclosure provides a method of producing an anti-CD3 antibody or a functional fragment thereof comprising culturing a host cell comprising an expression vector encoding the antibody or functional fragment in culture medium under conditions sufficient to cause the host cell to express the antibody or fragment capable of binding CD3.

In another aspect, the present disclosure provides a method of producing a bispecific, multivalent binding protein capable of binding ROR1 and CD3, specifically a FIT-Ig or MAT-Fab binding protein binding ROR1 and CD3, comprising culturing a host cell comprising an expression vector encoding the FIT-Ig or MAT-Fab binding protein in culture medium under conditions sufficient to cause the host cell to express the binding protein capable of binding ROR1 and CD3. The proteins produced by the methods disclosed herein can be isolated and used in various compositions and methods described herein.

### Uses of Antibodies and Binding Proteins

Given their ability to bind to human ROR1 and/or CD3, the antibodies described herein, functional fragments thereof, and bispecific multivalent binding proteins described herein can be used to detect ROR1 or CD3, or both, e.g., in a biological sample containing cells that express one or both of those target antigens. The antibodies, functional fragments, and binding proteins of the present disclosure can be used in a conventional immunoassay, such as an enzyme linked immunosorbent assay (ELISA), a radioimmunoassay (RIA), or tissue immunohistochemistry. The present disclosure provides a method for detecting ROR1 or CD3 in a biological sample comprising contacting a biological sample with an antibody, antigen-binding portion thereof, or binding protein of the present disclosure and detecting whether binding to a target antigen occurs, thereby detecting the presence or absence of the target in the biological sample. The antibody, functional fragment, or binding protein may be directly or indirectly labeled with a detectable substance to facilitate detection of the bound or unbound antibody/fragment/binding protein. Suitable detectable substances include various enzymes, prosthetic groups, fluorescent materials, luminescent materials, and radioactive materials. Examples of suitable enzymes include horseradish peroxidase, alkaline phosphatase, β-galactosidase, or acetylcholinesterase. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S ⁹⁰Y, ⁹⁹Tc, ¹¹¹In, ¹²⁵I, ¹³¹I, ¹⁷⁷Lu, ¹⁶⁶Ho, or ¹⁵³Sm.

In some embodiments, the antibodies, functional fragments thereof, of the present disclosure are capable of neutralizing human ROR1 activity both in vitro and in vivo. Accordingly, the antibodies, functional fragments thereof, of the present disclosure can be used to inhibit human ROR1 activity, e.g., inhibit cell signaling mediated by ROR1 in a cell culture containing ROR1-expressing cells, in human subjects, or in other mammalian subjects having ROR1 with which an antibody, functional fragment thereof, or binding protein of the present disclosure cross-reacts.

In another embodiment, the present disclosure provides an antibody or bispecific binding protein of the present disclosure for use in treating a subject suffering from a disease or disorder in which ROR1 activity is detrimental, wherein the antibody or binding protein is administered to the subject such that activity mediated by ROR1 in the subject is reduced. As used herein, the term “a disorder in which ROR1 activity is detrimental” is intended to include diseases and other disorders in which the interaction of ROR1 with its ligand (Wnt-5A) in a subject suffering from the disorder is either responsible for the pathophysiology of the disorder or is a factor that contributes to a worsening of the disorder. Accordingly, a disorder in which ROR1 activity is detrimental is a disorder in which inhibition of ROR1 activity is expected to alleviate the symptoms and/or progression of the disorder. In one embodiment, an anti-ROR1 antibody, functional fragment thereof, of the present disclosure is used in a method that inhibits the growth or survival of malignant cells, or reduces the tumor burden.

In some embodiments, the bispecific binding proteins (FIT-Ig or MAT-Fab) of the present disclosure are capable of redirecting T cell cytotoxicity towards ROR-expressing cells both in vitro and in vivo. Accordingly, the bispecific binding proteins of the present disclosure can be used to inhibit the growth or expansion of ROR1-expressing malignant cells, in human subjects, or in other mammalian subjects having ROR1 with which an antibody, functional fragment thereof, or bispecific binding protein of the present disclosure cross-reacts.

In another embodiment, the present disclosure provides a CD3/ROR1 bispecific (FIT-Ig or MAT-Fab) binding protein for use in treating an ROR1-expressing malignancy in a subject, wherein the binding protein is administered to the subject. In some embodiments, the malignancy is a solid tumor or hematopoietic malignancy.

The antibodies (including functional fragments thereof) and binding proteins of the present disclosure can be incorporated into pharmaceutical compositions suitable for administration to a subject. Typically, the pharmaceutical composition comprises an antibody or binding protein of the present disclosure and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols (such as, mannitol or sorbitol), or sodium chloride in the composition. Pharmaceutically acceptable carriers may further comprise minor amounts of auxiliary substances such as wetting or emulsifying agents, preservatives, or buffers, which enhance the shelf life or effectiveness of the antibody or binding protein present in the composition. A pharmaceutical composition of the present disclosure is formulated to be compatible with its intended route of administration.

The method of the present disclosure may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the primary active ingredient may be in powder form for constitution with a suitable vehicle (e.g., sterile pyrogen-free water) before use.

The use of the present disclosure may include administration of compositions formulated as depot preparations. Such long acting formulations may be administered by implantation (e.g., subcutaneously or intramuscularly) or by intramuscular injection. For example, the compositions may be formulated with suitable polymeric or hydrophobic materials (e.g., as an emulsion in an acceptable oil) or ion exchange resins, or as sparingly soluble derivatives (e.g., as a sparingly soluble salt).

An antibody, functional fragment thereof, or binding protein of the present disclosure also can be administered with one or more additional therapeutic agents useful in the treatment of various diseases. Antibodies, functional fragments thereof, and binding proteins described herein can be used alone or in combination with an additional agent, e.g., an additional therapeutic agent, the additional agent being selected by the skilled artisan for its intended purpose. For example, the additional agent can be a therapeutic agent art-recognized as being useful to treat the disease or condition being treated by the antibody or binding protein of the present disclosure. The additional agent also can be an agent that imparts a beneficial attribute to the therapeutic composition, e.g., an agent that affects the viscosity of the composition.

### Methods for Treatment and Medical Uses

In one embodiment, the present disclosure provides methods for treating a disorder in which ROR1-mediated signaling activity is associated or detrimental (such as ROR⁺ solid tumors or hematopoietic malignancies) in a subject in need thereof, the method comprising administering to the subject an anti-ROR1 antibody or ROR1-binding fragment thereof as described herein, wherein the antibody or binding fragment is capable of binding ROR1 and inhibiting ROR1-mediated signaling in a cell expressing ROR1. In another embodiment, the present disclosure provides use of an effective amount of an anti-ROR1 antibody or antigen-binding fragment thereof described herein in the treatment of such a disorder. In another embodiment, the present disclosure provides use of an anti-ROR1 antibody or antigen-binding fragment thereof described herein in the manufacture of a composition for the treatment of such a disorder. In another embodiment, the present disclosure provides an anti-ROR1 antibody or antigen-binding fragment thereof described herein for use in the treatment of such a disorder.

In a further embodiment of the method or use described herein, an anti-ROR1 antibody or antigen binding fragment of the present disclosure binds ROR1, and comprises a VH domain comprising, consisting essentially of, or consisting of the sequence of SEQ ID NO: 10 or 21, and a VL domain comprising, consisting essentially of, or consisting of the sequence of SEQ ID NO: 13.

In another embodiment, the present disclosure provides methods for treating a disorder in which ROR1-mediated signaling activity is associated or detrimental (such as ROR⁺ solid tumors or hematopoietic malignancies) in a subject in need thereof, the method comprising administering to the subject a bispecific FIT-Ig or MAT-Fab binding protein capable of binding CD3 and ROR1 as described herein, wherein the binding protein is capable of binding CD3 and ROR1 and inducing redirected T-cell cytotoxicity to ROR1-expressing tumor cells. In another embodiment, the present disclosure provides use of an effective amount of the bispecific FIT-Ig or MAT-Fab binding protein described herein in the treatment of such a disorder. In another embodiment, the present disclosure provides use of the bispecific FIT-Ig or MAT-Fab binding protein described herein in the manufacture of a composition for the treatment of such a disorder. In another embodiment, the present disclosure provides the bispecific FIT-Ig or MAT-Fab binding protein described herein for use in the treatment of such a disorder.

In a further embodiment of the method or use described herein, a FIT-Ig binding protein of the present disclosure binds ROR1 and CD3 and is comprised of a first polypeptide chain comprising, consisting essentially of, or consisting of the sequence of SEQ ID NO:34 or 37; a second polypeptide chain comprising, consisting essentially of, or consisting of the sequence of SEQ ID NO:35; and a third polypeptide chain comprising, consisting essentially of, or consisting of the sequence of SEQ ID NO:36. In a further embodiment, a MAT-Fab binding protein of the present disclosure binds ROR1 and CD3 and is comprised of a first polypeptide chain comprising, consisting essentially of, or consisting of the sequence of SEQ ID NO:38 or 40; a second polypeptide chain comprising, consisting essentially of, or consisting of the sequence of SEQ ID NO:35; a third polypeptide chain comprising, consisting essentially of, or consisting of the sequence of SEQ ID NO:36; and a fourth polypeptide chain comprising, consisting essentially of, or consisting of the sequence of SEQ ID NO:39.

In some embodiments, the disorders which can be treated with the antibody or binding protein according to the present disclosure include various hematopoietic and solid malignancies expressing ROR1 on the cell surface of the malignant cells. In another embodiment, the antibody or the binding protein inhibits the growth or survival of malignant cells. In another embodiment, the antibody or the binding protein reduces the tumor burden. In another embodiment, the cancer is breast cancer such as triple-negative breast adenocarcinoma, or leukemia such as chronic lymphocytic leukemia (CLL).

Methods of treatment described herein may further comprise administering to a subject in need thereof, of additional active ingredient, which is suitably present in combination with the present antibody or binding protein for the treatment purpose intended, for example, another drug having ant-tumor activity. In a method of treatment of the present disclosure, the additional active ingredient may be incorporated into a composition comprising an antibody or binding protein of the present disclosure, and the composition administered to a subject in need of treatment. In another embodiment, a method of treatment of the present disclosure may comprise a step of administering to a subject in need of treatment an antibody or binding protein described herein and a separate step of administering the additional active ingredient to the subject before, concurrently, or after the step of administering to the subject an antibody or binding protein of the present disclosure.

Having now described the present disclosure in detail, the same will be more clearly understood by reference to the following examples, which are included for purposes of illustration only and are not intended to be limiting of the present disclosure.

### EXAMPLES

To obtain ROR1 targeting monoclonal antibodies with improved properties, anti-ROR1 antibodies were generated using conventional hybridoma technology. Antibody ROR1-mAb004, which binds to ROR1 at the C-terminus of the ROR1 Ig-like domain, was then selected and characterized. The ROR1-mAb004 sequence was further humanized by the conventional CDR grafting method. Humanized sequences were designed. Some of these sequences were expressed as recombinant FIT-Ig and characterized for their binding affinity.

A FIT-Ig protein FIT1007-12B-17 was constructed, and its MAT-Fab counterpart, MAT1007-12B-17, as well as its low CD3 affinity comparator, FIT1007-12B-18, were also generated. In general, when having the same Ig variable sequences, FIT-Ig format showed superior in vitro tumor cell killing efficacy and higher cytokine release than MAT-Fab. Reduced CD3 affinity also led to reduced redirected T cell cytotoxicity (RTCC) efficacy.

Both FIT-Ig and MAT-Fab showed ROR1 target dependent activation of T cells in a cocultured report gene assay. This suggests that T cells may not be efficiently activated when the target ROR1 is not present. This phenomenon is consistent with the CD3 binding activity difference between FIT-Ig and its parental CD3 monoclonal antibody.

FIT-Ig and MAT-Fab showed potent in vivo efficacy in a triple negative breast cancer xenograft model.

### Example 1. Generation of Anti-ROR1 Antibodies

Anti-ROR1 antibodies were obtained by immunizing Balb/c or SJL mice with Q30-Y406 of human ROR1, a recombinant human ROR1 extracellular domain (UniProt Identifier: Q01973-1):

>HUMAN_ROR1_ECD
(SEQ ID NO: 41)
QETELSVSAELVPTSSWNISSELNKDSYLTLDEPMNNITTSLGQTAELH
CKVSGNPPPTIRWEKNDAPVVQEPRRLSFRSTIYGSRLRIRNLDTTDTG
YFQCVATNGKEVVSSTGVLFVKFGPPPTASPGYSDEYEEDGFCQPYRGI
ACARFIGNRTVYMESLHMQGEIENQITAAFTMIGTSSHLSDKCSQFAIP
SLCHYAFPYCDETSSVPKPRDLCRDECEILENVLCQTEYIFARSNPMIL
MRLKLPNCEDLPQPESPEAANCIRIGIPMADPINKNHKCYNSTGVDYRG
TVSVTKSGRQCQPWNSQYPHTHTFTALRFPELNGGHSYCRNPGNQKEAP
WCFTLDENFKSDLCDIPACDSKDSKEKNKMEILY

Mice were immunized at 2-week intervals and monitored for serum titer once a week after the second injection. After 4 to 6 immunizations, splenocytes were harvested and fused with mouse myeloma cells to form hybridoma cell lines. Fusion products were plated in selection media containing hypoxanthine-aminopterin-thymidine (HAT) in 96-well plates at a density of 1×10⁵ spleen cells per well. Seven to ten days post-fusion, macroscopic hybridoma colonies were observed. Supernatants of hybridoma cells were then screened and selected to identify cell lines producing ROR1-specific mouse antibodies. Upon preliminary characterization, one anti-ROR1 antibody, ROR1-mAb004, was selected and sequenced.

### Example 1.1 Heavy and Light Chain Variable Region Sequences

To amplify heavy and light chain variable regions, total RNA of each hybridoma clone was isolated from more than 5×10⁶ cells with TRIzol™ RNA extraction reagent (Invitrogen, Cat. #15596018). cDNA was synthesized using an Invitrogen™ SuperScript™ III First-Strand Synthesis SuperMix kit (ThermoFisher Scientific Cat. #18080) following manufacturer's instructions, and the cDNAs encoding the variable regions for light and heavy mouse immunoglobulin chains were amplified using a MilliporeSigma™ Novagen™ Mouse Ig-Primer Set (Fisher Scientific Cat. #698313). PCR products were analyzed by electrophoresis on a 1.2% agarose gel with SYBR™ Safe DNA gel stain (ThermoFisher Cat. #S33102). DNA fragments with correct size were purified using a NucleoSpin® Gel and PCR Clean-up kit (Macherey-Nagel, Cat. #740609) according to manufacturer's instructions and were subcloned into pMD18-T vector individually. Fifteen colonies from each transformation were selected and sequences of insert fragments were analyzed by DNA sequencing. The protein sequences of murine mAb variable regions were analyzed by sequence homology alignment.

The variable domain sequence for the selected anti-ROR1 antibody is set out in the table below. Complementarity determining regions (CDRs) are underlined based on Kabat numbering.

TABLE 1
Amino acid sequences of variable regions of anti-ROR1 antibody

| Antibody | domain | SEQ ID NO. | amino acid sequence |
|---|---|---|---|
| ROR1- | VH | 8 | QVQLQQSGPELVKPGASVKISCKASGYAFSRSWMNWVKQR |
| mAb004 | | | PEKGLEWIGRIYPGNGDIKYNGNFKGKATLTADKSSSTAY |
| | | | MQLSSLTSEDSAVYFCAHIYYDFYYALDYWGQGTSVTVSS |
| | VL | 9 | DIQLTQSPSSLSASLGGKVTITCKASQDINKYITWYQHKP |
| | | | GKGPRLLIHYTSTLQPGIPSRESGSGSGRDYSFSISNLEP |
| | | | EDIATYYCLQYDSLLWTFGGGTKLEIK |

### Example 1.2 Binding Kinetics of Anti-ROR1 Antibodies

Binding affinities and kinetics constants of anti-ROR1 antibodies were determined at 25° C. using an Octet®RED96 biolayer interferometry (Pall ForteBio LLC) following standard procedures. Briefly, Anti-Mouse IgG Fc Capture (AMC) Biosensors were used to capture purified anti-ROR1 antibodies. Sensors were then dipped into solutions containing recombinant human ROR1-ECD protein to detect target protein binding to the captured antibodies. Kinetics constants were determined by processing and fitting data to a 1:1 binding model using Fortebio analysis software. Shown below in Table 2 are the results obtained for ROR1-mAb004 in comparison with two previously described anti-ROR1 monoclonal antibodies, ROR1-Tab1 is clone R12 as described in WO2014167022, and ROR1-Tab2 is clone D10 as described in WO2012097313.

TABLE 2
Binding kinetics of anti-ROR1 monoclonal antibodies

| Sample ID | KD (M) | kon(1/Ms) | kdis(1/s) |
|---|---|---|---|
| ROR1-mAb004 | 1.85E−08 | 9.25E+04 | 1.71E−03 |
| ROR1-Tab1 | 1.28E−09 | 5.17E+05 | 6.60E−04 |
| ROR1-Tab2 | 9.88E−08 | 3.25E+05 | 3.21E−02 |

### Example 1.3 Cell Surface Binding Characterization of Anti-ROR1 Antibodies

The binding specificity and potency of anti-ROR1 antibodies were characterized by protein ELISA and flow cytometry analysis of cell surface binding. Binding EC50s were calculated and are shown in Table 3 below. Briefly, binding properties of the anti-ROR1 antibodies were measured with ELISA as follows: recombinant ROR1-ECD protein was coated at 1 μg/mL on 96-well plates at 4° C. overnight. Plates were washed once with washing buffer (PBS containing 0.05% Tween 20) and blocked with ELISA blocking buffer (1% BSA in PBS containing 0.05% Tween 20) at room temperature for 2 hours. Anti-ROR1 antibodies were then added and incubated at 37° C. for 1 hour. Plates were washed three times with washing buffer. HRP labeled anti-mouse IgG secondary antibody (Sigma, Cat. #A0168) was added and the plates were incubated at 37° C. for 30 minutes then washed 5 times in washing buffer. 100 μl of tetramethylbenzidine (TMB) chromogenic solution was added to each well. Following color development, the reaction was stopped with 1 Normal HCl and absorbance at 450 nm was measured on a Varioskan™ LUX microplate reader (ThermoFisher Scientific). Binding signals were plotted against antibody concentration with GraphPad Prism 6.0 software and EC50s were calculated accordingly. The results are shown in FIG. 1. FIG. 1 shows the ROR1-ECD protein binding activities of monoclonal antibodies ROR1-mAb004 and ROR1-Tab1, and irrelevant mIgG1 was used as negative control.

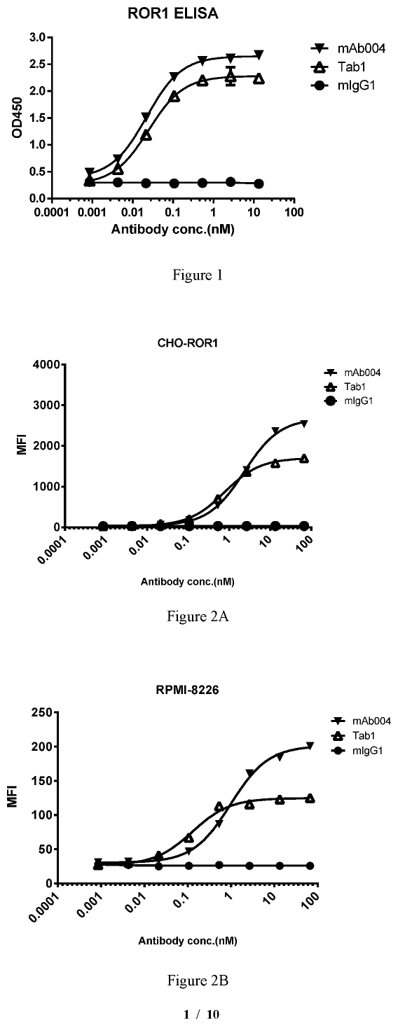

Cell binding activity of anti-ROR1 antibodies were measured with human ROR1 transfected CHO cell line (CHO-ROR1) and ROR1-expressing myeloma cell line (RPMI8226). Briefly, 5×10⁵ cells were seeded into each well of a 96-well plate. Cells were centrifuged at 400 g for 5 minutes and supernatants were discarded. For each well, 100 μl of serially diluted antibodies were then added and mixed with the cells. After 40 minutes incubation at 4° C., plates were washed several times to remove excess antibodies. Secondary fluorochrome-conjugated goat anti-mouse IgG antibody was then added and incubated with cells at room temperature for 20 minutes. After another round of centrifugation and a washing step, cells were resuspended in FACS buffer for reading on a CytoFLEX Flow Cytometer (Beckman Coulter). Median Fluorescence Intensity (MFI) readouts were plotted against antibody concentration and analyzed with GraphPad Prism 6.0 software. The results shown in FIGS. 2A-B illustrate the binding activities of anti-ROR1 monoclonal antibodies ROR1-mAb004 and ROR1-Tab1 to ROR1 expressing cells. An irrelevant mIgG1 was used as negative control.

TABLE 3
Binding EC50 of anti-ROR1 monoclonal antibodies

EC50 (nM)

| Sample ID | ROR1-ECD | CHOK1-ROR1 | RPMI-8226 |
|---|---|---|---|
| ROR1-mAb004 | 0.022 | 2.306 | 0.983 |
| ROR1-Tab1 | 0.025 | 0.774 | 0.128 |

### Example 1.4 Internalization Characterization of Anti-ROR1 Antibodies

The binding internalization of anti-ROR1 antibodies were characterized with ROR1-expressing myeloma cell line RPMI8226. Cells were harvested and resuspended in FACS buffer at density of 3 million per mL. Diluted antibodies were added to the tubes and incubated for 30 min at 4° C. After the first incubation, cells were washed three times with cold PBS to remove unbound antibody. Then the cells of each antibody treatment were split into two groups, for “control” and “internalization”, respectively. Cells in the “internalization” group were resuspended in pre-warmed medium and incubated at 37° C. for 2 hours to allow internalization, while cells in the “control” group were kept at 4° C. for the same period. After the second incubation, cells were washed once with cold PBS and incubated with fluorescein labeled secondary antibody for 30 min at 4° C. After another round of centrifugation and washing step, cells were resuspended in FACS buffer for reading on a CytoFLEX Flow Cytometer (Beckman Coulter). An irrelevant mouse IgG control (MFI_{background}) was used for background calibration. The difference between the MFI readout of “control” and that of “internalization” (ΔMFI) reflects the internalization of ROR1 antibodies, and such difference in relative to calibrated MFI of “control” reflects percentage of antibody internalization, which is calculated as following and summarized below in Table 4,

Percentage of internalization (ΔMFI)=[1−(MFI_{internalization}−MFI_{background})/(MFI_{control}−MFI_{background})]×100%

TABLE 4
Internalization percentage of anti-ROR1 monoclonal antibodies.

| Sample ID | Percentage of Internalization |
|---|---|
| ROR1-mAb004 | 11.58% |
| ROR1-Tab1 | −3.23% |
| ROR1-Tab2 | 29.94% |

### Example 1.5 Epitope Binning of Anti-ROR1 Antibodies

The binding epitope of ROR1 antibodies were identified with a competition ELISA. Briefly, 96 well plates were coated with 1 ug/mL purified antibodies and incubated overnight at 4° C. After washing with PBS containing 0.05% Tween 20, plates were blocked with blocking buffer (PBS containing 0.05% Tween 20 and 2% BSA) at 37° C. for 2 hours. Biotinylated human ROR1-ECD protein pre-mixed with ROR1 antibody (sample) or irrelevant mouse IgG (baseline) was added into plate wells and incubated at 37° C. for 1 hour before being washed 3 times. Streptavidin-HRP (1:5000 dilution) was then added into each well and incubated at 37° C. for 1 hour before being washed another 3 times. Tetramethylbenzidine (TMB) chromogenic solution was added for color development for 5 minutes then the reaction was stopped with 1M HCl. Absorbance at 450 nm (OD₄₅₀) was measured on a microplate reader. The OD450_{baseline} represents the level of human ROR1-ECD binding to ROR1 antibodies at absence of competition, while the difference between OD450_{baseline} and OD450ₛₐₘₚₗₑ reflects the competition between the ROR1 antibody coated on plate and the antibody in solution. The inhibition percentage was calculated by following equation:

Inhibition %=(1−OD450ₛₐₘₚₗₑ/OD450_{baseline})×100%

Table 5 below shows results of the competition ELISA in terms of percent inhibition, indicating ROR1-mAb004 competes with ROR1-Tab2, but does not compete with ROR1-Tab 1.

TABLE 5
Competition ELISA result of anti-ROR1 monoclonal antibodies.

Coating

| Competition | ROR1-Tab1 | ROR1-mAb004 | ROR1-Tab2 |
|---|---|---|---|
| ROR1-Tab1 | 95% | −54% | −84% |
| ROR1-mAb004 | −22% | 93% | 92% |
| ROR1-Tab2 | −21% | 56% | 92% |

### Example 2. Humanization Design of ROR1-mAb004

The ROR1-mAb004 variable region genes were employed for humanization design. In the first step of this process, the amino acid sequences of the VH and VL domains of ROR1-mAb004 were compared against the available database of human Ig V-gene sequences in order to find the overall best-matching human germline Ig V-gene sequences. Additionally, the framework 4 segment of the VH or VL was compared against the J-region database to find the human framework having the highest homology to the murine VH and VL regions, respectively. For the light chain, the closest human V-gene match was the O18 gene; and for the heavy chain, the closest human match was the VH1-69 gene. Humanized variable domain sequences were then designed where the CDR-L1, CDR-L2, and CDR-L3 of the VL domain of the ROR1-mAb004 light chain were grafted onto framework sequences of the 018 gene with JK4 framework 4 sequence after CDR-L3, respectively; and the CDR-H1, CDR-H2, and CDR-H3 of the VH domain of the ROR1-mAb004 heavy chain were grafted onto framework sequences of the VH1-69 with JH6 framework 4 sequence after CDR-H3. A three-dimensional Fv model of ROR1-mAb004 was then generated to determine if there were any framework positions where mouse amino acids were involved in supporting loop structures or the VH/VL interface. These residues in humanized sequences could be back mutated to mouse residues at the same positions to retain affinity/activity. Several desirable back mutations were identified for ROR1-mAb004 VH and VL, and alternative VH and VL designs were constructed, as shown in Table 6 below.

In addition, 4 mouse VH sequences with different point mutations were also designed and shown in the last 4 VH sequences in Table 6, to avoid the potential asparagine deamidation introduced by the two “NG” (Asn-Gly) amino acids in the CDR-H2 of ROR1-mAb004. See, for example, Qingrong Yan et al., (2018) Structure Based Prediction of Asparagine Deamidation Propensity in Monoclonal Antibodies, mAbs, 10:6, 901-912, for asparagine deamidation induced by “NG” (Asn-Gly) amino acids in the CDR-H2 of an antibody and the effects thereof on the stability of the antibody.

TABLE 6
VH/VL humanization and point-mutation design for ROR1-mAb004

| Humanized ROR1- | | |
|---|---|---|
| mAb004 VH/VL | SEQ ID | |
| Identifier | NO. | Amino acid sequence |
| ROR1-mAb004VH.1a | 10 | EVQLVQSGAEVKKPGSSVKVSCKASGYTFSRSWMNWVRQAPG |
| | | QGLEWMGRIYPGNGDIKYNGNFKGRVTITADKSTSTAYMELS |
| | | SLRSEDTAVYYCAHIYYDFYYALDYWGQGTTVTVSS |
| ROR1-mAb004VH.1b | 11 | EVQLVQSGAEVKKPGSSVKVSCKASGYTFSRSWMNWVRQAPG |
| | | QGLEWIGRIYPGNGDIKYNGNEKGRATITADKSTSTAYMELS |
| | | SLRSEDTAVYYCAHIYYDFYYALDYWGQGTTVTVSS |
| ROR1-mAb004VH.1c | 12 | EVQLVQSGAEVKKPGSSVKVSCKASGYTFSRSWMNWVKQAPG |
| | | QGLEWIGRIYPGNGDIKYNGNFKGKATITADKSTSTAYMELS |
| | | SLRSEDTAVYYCAHIYYDFYYALDYWGQGTTVTVSS |
| ROR1-mAb004VK.1a | 13 | DIQMTQSPSSLSASVGDRVTITCKASQDINKYITWYQQKPGK |
| | | APKLLIYYTSTLQPGVPSRFSGSGSGTDYTFTISSLQPEDIA |
| | | TYYCLQYDSLLWTFGGGTKVEIK |
| ROR1-mAb004VK.1b | 14 | DIQMTQSPSSLSASVGDRVTITCKASQDINKYITWYQQKPGK |
| | | APKLLIHYTSTLQPGVPSRFSGSGSGRDYTFTISSLQPEDIA |
| | | TYYCLQYDSLLWTFGGGTKVEIK |
| ROR1-mAb004VK.1c | 15 | DIQLTQSPSSLSASVGDRVTITCKASQDINKYITWYQQKPGK |
| | | APKLLIYYTSTLQPGVPSRFSGSGSGRDYTFTISSLQPEDIA |
| | | TYYCLQYDSLLWTFGGGTKVEIK |
| ROR1-mAb004VK.1d | 16 | DIQLTQSPSSLSASVGDRVTITCKASQDINKYITWYQQKPGK |
| | | APKLLIHYTSTLQPGIPSRFSGSGSGRDYTFTISSLQPEDIA |
| | | TYYCLQYDSLLWTFGGGTKVEIK |
| ROR1-mAb004 VH | | |
| Identifier, with point | SEQ ID | |
| mutations in CDR-H2 | NO. | Amino acid sequence |
| ROR1-mAb004VH(AA) | 17 | QVQLQQSGPELVKPGASVKISCKASGYAFSRSWMNWVKQRPE |
| | | KGLEWIGRIYPGNADIKYNANFKGKATLTADKSSSTAYMQLS |
| | | SLTSEDSAVYFCAHIYYDFYYALDYWGQGTSVTVSS |
| ROR1-mAb004VH(QQ) | 18 | QVQLQQSGPELVKPGASVKISCKASGYAFSRSWMNWVKQRPE |
| | | KGLEWIGRIYPGQGDIKYQGNFKGKATLTADKSSSTAYMQLS |
| | | SLTSEDSAVYFCAHIYYDFYYALDYWGQGTSVTVSS |
| ROR1-mAb004VH(AQ) | 19 | QVQLQQSGPELVKPGASVKISCKASGYAFSRSWMNWVKQRPE |
| | | KGLEWIGRIYPGNADIKYQGNFKGKATLTADKSSSTAYMQLS |
| | | SLTSEDSAVYFCAHIYYDFYYALDYWGQGTSVTVSS |
| ROR1-mAb004VH(QA) | 20 | QVQLQQSGPELVKPGASVKISCKASGYAFSRSWMNWVKQRPE |
| | | KGLEWIGRIYPGQGDIKYNANFKGKATLTADKSSSTAYMQLS |
| | | SLTSEDSAVYFCAHIYYDFYYALDYWGQGTSVTVSS |

Note:
Back mutated framework amino acid residues in humanized antibodies, and CDR-H2 point mutations in chimeric antibodies, are indicated with double underscore.

### Example 3. Generation and Characterization of Humanized Anti-CD3 Antibody

Hybridoma-produced anti-CD3 monoclonal antibody mAbCD3-001 was generated and selected using conventional hybridoma technology, then humanized by the conventional CDR grafting method. Back mutations were then introduced in the humanized VH sequences, and an NS mutation was made to replace NA in the humanized kappa chain in order to remove asparagine deamidation liability (detailed description provided in PCT/CN/120991, which is incorporated herein by reference in its entirety). The resultant humanized VH and VL constructs are shown in Table 7 (below).

TABLE 7
CD3 antibodies variable region sequences

| Anti-CD3 VH/VL | SEQ | |
|---|---|---|
| Identifier | ID NO. | Amino acid sequence |
| EM0006-01vh.1h | 22 | EVQLVQSGAEVKKPGASVKVSCKASGESFTNYYVHWMRQA |
| | | PGQGLEWMGWISPGSDNTKYNEKFKGRVTMTRDTSISTAY |
| | | MELSRLRSDDTAVYYCARDDYGNYYFDYWGQGTTVTVSS |
| EM0006-01vh.1g | 23 | EVQLVQSGAEVKKPGASVKVSCKASGESFTNYYVHWMRQA |
| | | PGQGLEWIGWISPGSDNTKYNEKFKGRVTLTADTSISTAY |
| | | MELSRLRSDDTAVYYCARDDYGNYYFDYWGQGTTVTVSS |
| EM0006- | 24 | DIVMTQSPDSLAVSLGERATINCKSSQSLLNARTRKNYLA |
| 01vK.1(s32aa) | | WYQQKPGQPPKLLIYWASTRESGVPDRESGSGSGTDETLT |
| | | ISSLQAEDVAVYYCKQSYILRTFGGGTKVEIK |

The pairing of the human VH and the human VK sequences created 2 humanized antibodies, designated HuEM0006-01-24 (with VH/VL pair of SEQ ID NOs: 22 and 24) and HuEM0006-01-27 (with VH/VL pair of SEQ ID NOs: 23 and 24) (Table 7). The recombinant humanized mAbs were transiently expressed in HEK293 cells and purified by Protein A chromatography.

The binding activities of the humanized anti-CD3 antibodies were tested via flow cytometry with the human CD3-expressing Jurkat T cell line. 5×10⁵ Jurkat cells in FACS buffer were seeded into each well of a 96-well plate. Cells were centrifuged at 400 g for 5 minutes and supernatants were discarded. For each well, 100 IA of serially diluted antibodies were then added and mixed with the cells. After 40 minutes of incubation at 4° C., plates were washed several times to remove excess antibodies. Secondary fluorochrome-conjugated antibody (Alexa Fluor® 647 goat anti-human IgG1 H&L; Jackson ImmunoResearch, Cat. #109-606-170) was then added and incubated with cells at room temperature for 20 minutes. After another round of centrifugation and a washing step, cells were resuspended in FACS buffer for reading on a CytoFLEX Flow Cytometer (Beckman Coulter). Median Fluorescence Intensity (MFI) readouts were plotted against antibody concentration and analyzed with GraphPad Prism 5.0 software. The antibody HuEM0006-01-24 exhibited higher CD3 binding affinity than the antibody HuEM0006-01-27.

### Example 4. Generation of ROR1/CD3 FIT-Ig

A group of FIT-Ig proteins recognizing both human ROR1 and human CD3 were constructed utilizing VH/VL sequences in Table 6 as anti-ROR1 moiety, VH/VL sequences in Table 7 as anti-CD3 moiety, and human constant region sequences in Table 8.

TABLE 8
Human IoG constant region sequences

| Constant | SEQ | |
|---|---|---|
| Region | ID NO. | Amino acid sequence |
| CH1-hinge- | 31 | ASTKGPSVFPLAPSSKSTSGGTAALGCLVKDYFPEPVTVSWN |
| CH2-CH3 | | SGALTSGVHTFPAVLQSSGLYSLSSVVTVPSSSLGTQTYICN |
| (human constant | | VNHKPSNTKVDKKVEPKSCDKTHTCPPCPAPEAAGGPSVELE |
| IgG1 with | | PPKPKDTLMISRTPEVTCVVVDVSHEDPEVKENWYVDGVEVH |
| L234A/L235A | | NAKTKPREEQYNSTYRVVSVLTVLHQDWLNGKEYKCKVSNKA |
| mutation) | | LPAPIEKTISKAKGQPREPQVYTLPPSREEMTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK |
| CL | 32 | RTVAAPSVFIFPPSDEQLKSGTASVVCLLNNFYPREAKVQWK |
| (human constant | | VDNALQSGNSQESVTEQDSKDSTYSLSSTLTLSKADYEKHKV |
| kappa) | | YACEVTHQGLSSPVTKSENRGEC |
| CH1 | 33 | ASTKGPSVFPLAPSSKSTSGGTAALGCLVKDYFPEPVTVSWN |
| | | SGALTSGVHTFPAVLQSSGLYSLSSVVTVPSSSLGTQTYICN |
| | | VNHKPSNTKVDKKVEPKSC |

FIT-Ig molecules were constructed following the general procedures described in PCT Publication WO 2015/103072. Each FIT-Ig consisted of three polypeptide chains having the following structures:
Chain #1 (long chain): VL_{A}-CL-VH_{B}-CH1-hinge-CH2-CH3;
Chain #2 (first short chain): VH_{A}-CH1;
Chain #3 (second short chain): VL_{B}-CL;
wherein A stands for ROR1 and B stands for CD3, and VL_{ROR1} is the light chain variable domain of a humanized monoclonal antibody recognizing ROR1, VH_{CD3} is the heavy chain variable domain of a humanized monoclonal antibody recognizing CD3, VL_{CD3} is the light chain variable domain of a humanized monoclonal antibody recognizing CD3, VH_{ROR1} is the heavy chain variable domain of a humanized monoclonal antibody recognizing ROR1, each CL is a light chain constant domain (SEQ ID NO: 32), each CH1 is a first heavy chain constant domain (SEQ ID NO: 33), and CH1-hinge-CH2-CH3 is the C-terminal heavy chain constant region from CH1 through the terminus of the Fc region (SEQ ID NO: 31).

To construct the long chain vector, cDNA encoding the VL_{ROR1}-CL-VH_{CD3} segment was synthesized de novo and inserted into the multiple cloning site (MCS) of a vector including coding sequences for human CH1-hinge-CH2-CH3. In the resulting vector, the MCS sequence was eliminated during homologous recombination to ensure that all the domain fragments were in the correct reading frame. Similarly, to construct the first and second short chains, VH_{ROR1} and VL_{CD3} structural genes were de novo synthesized and inserted into the MCS of the appropriate vectors including coding segments for human CH1 and CL domains, respectively.

The pairing of the humanized VH and the humanized VL created the humanized ROR1/CD3 FIT-Ig binding proteins listed in Table 9 below. A chimeric antibody (FIT1007-12B) with parental mouse VH/VL of ROR1-mAb004 and human constant sequences was also produced as a positive control for humanized binding protein ranking.

TABLE 9
Production of FIT-Ig proteins with humanized anti-ROR1 VH/VL

| FIT-Ig Identifier | VH_{ROR1} | VL_{ROR1} | VH_{CD3} | VL_{CD3} |
|---|---|---|---|---|
| FIT1007-12B-1 | ROR1-mAb004VH.1a | ROR1-mAb004VK.1a | EM0006-01vh.1h | EM0006-01vk.1(s31aa) |
| FIT1007-12B-2 | ROR1-mAb004VH.1b | ROR1-mAb004VK.1a | EM0006-01vh.1h | EM0006-01vk.1(s31aa) |
| FIT1007-12B-3 | ROR1-mAb004VH.1c | ROR1-mAb004VK.1a | EM0006-01vh.1h | EM0006-01vk.1(s31aa) |
| FIT1007-12B-4 | ROR1-mAb004VH.1a | ROR1-mAb004VK.1b | EM0006-01vh.1h | EM0006-01vk.1(s31aa) |
| FIT1007-12B-5 | ROR1-mAb004VH.1b | ROR1-mAb004VK.1b | EM0006-01vh.1h | EM0006-01vk.1(s31aa) |
| FIT1007-12B-6 | ROR1-mAb004VH.1c | ROR1-mAb004VK.1b | EM0006-01vh.1h | EM0006-01vk.1(s31aa) |
| FIT1007-12B-7 | ROR1-mAb004VH.1a | ROR1-mAb004VK.1c | EM0006-01vh.1h | EM0006-01vk.1(s31aa) |
| FIT1007-12B-8 | ROR1-mAb004VH.1b | ROR1-mAb004VK.1c | EM0006-01vh.1h | EM0006-01vk.1(s31aa) |
| FIT1007-12B-9 | ROR1-mAb004VH.1c | ROR1-mAb004VK.1c | EM0006-01vh.1h | EM0006-01vk.1(s31aa) |
| FIT1007-12B-10 | ROR1-mAb004VH.1a | ROR1-mAb004VK.1d | EM0006-01vh.1h | EM0006-01vk.1(s31aa) |
| FIT1007-12B-11 | ROR1-mAb004VH.1b | ROR1-mAb004VK.1d | EM0006-01vh.1h | EM0006-01vk.1(s31aa) |
| FIT1007-12B-12 | ROR1-mAb004VH.1c | ROR1-mAb004VK.1d | EM0006-01vh.1h | EM0006-01vk.1(s31aa) |
| FIT1007-12B-13 | ROR1-mAb004VH(AA) | ROR1-mAb004VK | EM0006-01vh.1h | EM0006-01vk.1(s31aa) |
| FIT1007-12B-14 | ROR1-mAb004VH(QQ) | ROR1-mAb004VK | EM0006-01vh.1g | EM0006-01vk.1(s31aa) |
| FIT1007-12B-15 | ROR1-mAb004VH(AQ) | ROR1-mAb004VK | EM0006-01vh.1h | EM0006-01vk.1(s31aa) |
| FIT1007-12B-16 | ROR1-mAb004VH(QA) | ROR1-mAb004VK | EM0006-01vh.1h | EM0006-01vk.1(s31aa) |
| FIT1007-12B | ROR1-mAb004VH | ROR1-mAb004VK | EM0006-01vh.1h | EM0006-01vk.1(s31aa) |
| FIT1007-12B-18 | ROR1-mAb004VH.1a(AA) | ROR1-mAb004VK.1a | EM0006-01vh.1g | EM0006-01vk.1(s31aa) |

Recombinant FIT-Ig proteins listed in Table 10 were transiently expressed and purified as described herein. For each FIT-Ig construct, 3 plasmids respectively for the 3 polypeptide chains were co-transfected into HEK 293F cells. After approximately six days of post-transfection cell culture, the supernatants were harvested and subjected to Protein A affinity chromatography. The composition and purity of the purified antibodies were analyzed by size exclusion chromatography (SEC). Purified antibody, in PBS, was applied to a TSKgel SuperSW3000, 300×4.6 mm, SEC column (TOSOH). A DIONEX™ UltiMate 3000 HPLC instrument (Thermo Scientific) was used for SEC using UV detection at 280 nm and 214 nm. The expression and SEC-HPLC results were shown in Table 10 below.

The ROR1/CD3 FIT-Ig proteins were assayed for and ranked by dissociation rate constant (k_{off}, “off-rate”) using an Octet®RED96 biolayer interferometry (Pall FortéBio LLC). Anti-hIgG Fc Capture (AHC) Biosensors (Pall) were first exposed to antibody at a concentration of 100 nM for 30 seconds to capture antibody, then dipped into running buffer (1× pH 7.2 PBS, 0.05% Tween 20, 0.1% BSA) for 60 seconds to check baseline. Sensors with captured antibody were dipped into recombinant human ROR1 ECD protein at 10 ug/ml for 5 minutes to measure association, followed by dipped into running buffer for 1200 seconds to measure dissociation. The association and dissociation curves were fitted to a 1:1 Langmuir binding model using ForteBio Data Analysis software (Pall). Results are shown in Table 10 below. The off-rate ratios were calculated by the off-rate of antibody to that of FIT1007-12B. Lower ratio indicates slower dissociation of the antibody in comparison with the parental chimeric antibody FIT1007-12B.

TABLE 10
Generation and off-rate ranking of humanized and
chimeric ROR1-mAb004-related FIT-Ig proteins

| FIT-Ig | Expression | | Purity % | Off-rate |
|---|---|---|---|---|
| Identifier | Titer | | (SEC-HPLC) | Ratio |
| FIT1007-12B-1 | 27.16 | mg/L | 97.38 | 5.25% |
| FIT1007-12B-2 | 100.58 | mg/L | 95.4 | 7.87% |
| FIT1007-12B-3 | 65.75 | mg/L | 90.66 | 11.91% |
| FIT1007-12B-4 | 20.13 | mg/L | 97.63 | 35.89% |
| FIT1007-12B-5 | 80.55 | mg/L | 93.65 | 28.53% |
| FIT1007-12B-6 | 76.92 | mg/L | 93.35 | 25.81% |
| FIT1007-12B-7 | 27.23 | mg/L | 97.28 | 13.55% |
| FIT1007-12B-8 | 85.72 | mg/L | 94.29 | 15.37% |
| FIT1007-12B-9 | 69.35 | mg/L | 96.54 | 11.99% |
| FIT1007-12B-10 | 28.43 | mg/L | 97.28 | 36.73% |
| FIT1007-12B-11 | 64.12 | mg/L | 91.24 | 28.91% |
| FIT1007-12B-12 | 54.82 | mg/L | 92.82 | 25.16% |
| FIT1007-12B-13 | 14.56 | mg/L | 90.03 | 89.13% |
| FIT1007-12B-14 | No expression | | N/A | N/A |
| FIT1007-12B-15 | 1.66 | mg/L | 84.77 | No binding activity |
| FIT1007-12B-16 | 14.23 | mg/L | 91.51 | 139.13% |
| FIT1007-12B | 66.75 | mg/L | 97.13 | 100% |

The VH/VL humanization design of FIT1007-12B-1 was selected for the highest binding activity. Also, CDR-H2 point mutation design of FIT1007-12B-13 showed higher expression titer and binding activity comparing with other design. The mutation design of “ROR1-mAb004VH(AA)” (SEQ ID NO: 17) was selected for combination with the VH humanization design of “ROR1-mAb004VH.la” (SEQ ID NO: 10) to generate candidate molecules. The humanized VH sequence, namely ROR1-mAb004VH.la(AA), is shown below:

>ROR1-mAb004VH.1a(AA)
(SEQ ID NO: 21)
EVQLVQSGAEVKKPGSSVKVSCKASGYTFSRSWMNWVRQAPGQGLEWMG
RIYPGNADIKYNANEKGRVTITADKSTSTAYMELSSLRSEDTAVYYCAH
IYYDFYYALDYWGQGTTVTVSS

### Example 5. Construction and Expression of ROR1/CD3 FIT-Ig and MAT-Fab

The construction of FIT-Ig used the same method shown in Example 4. No linkers between the immunoglobulin domains were used. The complete sequences for the FIT-Ig binding proteins are provided in the sequence information in Table 11.

TABLE 11
Amino acid sequences of FIT-Ig component chains

| Polypeptide | SEQ ID NO. | Amino acid sequence |
|---|---|---|
| FIT1007-12B- | 34 | DIQMTQSPSSLSASVGDRVTITCKASQDINKYITWYQQKPGKAPK |
| 17 Chain #1 | | LLIYYTSTLQPGVPSRESGSGSGTDYTFTISSLQPEDIATYYCLQ |
| | | YDSLLWTFGGGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSENRGECEVQLVQSGAEV |
| | | KKPGASVKVSCKASGFSFTNYYVHWMRQAPGQGLEWMGWISPGSD |
| | | NTKYNEKFKGRVTMTRDTSISTAYMELSRLRSDDTAVYYCARDDY |
| | | GNYYFDYWGQGTTVTVSSASTKGPSVFPLAPSSKSTSGGTAALGC |
| | | LVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEA |
| | | AGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVDK |
| | | SRWQQGNVFSCSVMHEALHNHYTQKSLSLSPGK |
| FIT1007-12B- | 35 | EVQLVQSGAEVKKPGSSVKVSCKASGYTFSRSWMNWVRQAPGQGL |
| 17 Chain #2 | | EWMGRIYPGNADIKYNANFKGRVTITADKSTSTAYMELSSLRSED |
| | | TAVYYCAHIYYDFYYALDYWGQGTTVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSC |
| FIT1007-12B- | 36 | DIVMTQSPDSLAVSLGERATINCKSSQSLLNARTRKNYLAWYQQK |
| 17 Chain #3 | | PGQPPKLLIYWASTRESGVPDRESGSGSGTDETLTISSLQAEDVA |
| | | VYYCKQSYILRTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSENRGEC |
| FIT1007-12B- | 37 | DIQMTQSPSSLSASVGDRVTITCKASQDINKYITWYQQKPGKAPK |
| 18 Chain #1 | | LLIYYTSTLQPGVPSRESGSGSGTDYTFTISSLQPEDIATYYCLQ |
| | | YDSLLWTFGGGTKVEIKRTVAAPSVFIFPPSDEQLKSGTASVVCL |
| | | LNNFYPREAKVQWKVDNALQSGNSQESVTEQDSKDSTYSLSSTLT |
| | | LSKADYEKHKVYACEVTHQGLSSPVTKSFNRGECEVQLVQSGAEV |
| | | KKPGASVKVSCKASGFSFTNYYVHWMRQAPGQGLEWIGWISPGSD |
| | | NTKYNEKFKGRVTLTADTSISTAYMELSRLRSDDTAVYYCARDDY |
| | | GNYYFDYWGQGTTVTVSSASTKGPSVFPLAPSSKSTSGGTAALGC |
| | | LVKDYFPEPVTVSWNSGALTSGVHTFPAVLQSSGLYSLSSVVTVP |
| | | SSSLGTQTYICNVNHKPSNTKVDKKVEPKSCDKTHTCPPCPAPEA |
| | | AGGPSVFLFPPKPKDTLMISRTPEVTCVVVDVSHEDPEVKENWYV |
| | | DGVEVHNAKTKPREEQYNSTYRVVSVLTVLHQDWLNGKEYKCKVS |
| | | NKALPAPIEKTISKAKGQPREPQVYTLPPSREEMTKNQVSLTCLV |
| | | KGFYPSDIAVEWESNGQPENNYKTTPPVLDSDGSFFLYSKLTVDK |
| | | SRWQQGNVFSCSVMHEALHNHYTQKSLSLSPGK |
| FIT1007-12B- | 35 | EVQLVQSGAEVKKPGSSVKVSCKASGYTFSRSWMNWVRQAPGQGL |
| 18 Chain #2 | | EWMGRIYPGNADIKYNANFKGRVTITADKSTSTAYMELSSLRSED |
| | | TAVYYCAHIYYDFYYALDYWGQGTTVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSC |
| FIT1007-12B- | 36 | DIVMTQSPDSLAVSLGERATINCKSSQSLLNARTRKNYLAWYQQK |
| 18 Chain #3 | | PGQPPKLLIYWASTRESGVPDRESGSGSGTDETLTISSLQAEDVA |
| | | VYYCKQSYILRTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSENRGEC |

A group of ROR1/CD3 MAT-Fab proteins were also constructed with the same combination of VH/VL sequences following the procedure described in WO2018/035084. Each MAT-Fab consisted of four polypeptide chains having the following structures:
Chain #1 (long chain with “knob”): VL_{A}-CL-VH_{B}-CH1-hinge-CH2-CH3;
Chain #2 (first short chain): VH_{A}-CH1;
Chain #3 (second short chain): VL_{B}-CL;
Chain #4 (Fc “hole”): hinge-CH2-CH3;
wherein, chain #1 has a mutant human constant IgG1 with mutation S354C, T366W as a “knob”, chain #4 is the chain of Fc with mutation Y349C, T366S, L368A, Y407V as a “hole”, wherein A stands for ROR1 and B stands for CD3.

Following the similar cloning method as shown previously for FIT-Ig, the VH/VL genes of MAT-Fab polypeptide chains were produced synthetically and then respectively cloned into vectors containing respective constant domains. The complete sequences for the MAT-Fab proteins are provided in the sequence information in Table 12.

TABLE 12
Amino acid sequences of MAT-Fab component chains

| | SEQ ID | |
|---|---|---|
| Polypeptide | No. | Amino acid sequence |
| MAT1007- | 38 | DIQMTQSPSSLSASVGDRVTITCKASQDINKYITWYQQKPGKAPKLLIYYTSTL |
| 12B-17 chain | | QPGVPSRFSGSGSGTDYTFTISSLQPEDIATYYCLQYDSLLWTFGGGTKVEIKR |
| #1 | | TVAAPSVFIFPPSDEQLKSGTASVVCLLNNFYPREAKVQWKVDNALQSGNSQES |
| (VK-hck- | | VTEQDSKDSTYSLSSTLTLSKADYEKHKVYACEVTHQGLSSPVTKSENRGECEV |
| VH-hIgG1) | | QLVQSGAEVKKPGASVKVSCKASGFSFTNYYVHWMRQAPGQGLEWMGWISPGSD |
| | | NTKYNEKFKGRVTMTRDTSISTAYMELSRLRSDDTAVYYCARDDYGNYYEDYWG |
| | | QGTTVTVSSASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVS |
| | | NKALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMHEAL |
| | | HNHYTQKSLSLSPGK |
| MAT1007- | 35 | EVQLVQSGAEVKKPGSSVKVSCKASGYTFSRSWMNWVRQAPGQGLEWMGRIYPG |
| 12B-17 chain | | NADIKYNANFKGRVTITADKSTSTAYMELSSLRSEDTAVYYCAHIYYDFYYALD |
| #2 | | YWGQGTTVTVSSASTKGPSVFPLAPSSKSTSGGTAALGCLVKDYFPEPVTVSWN |
| (VH-CH1) | | SGALTSGVHTFPAVLQSSGLYSLSSVVTVPSSSLGTQTYICNVNHKPSNTKVDK |
| | | KVEPKSC |
| MAT1007- | 36 | DIVMTQSPDSLAVSLGERATINCKSSQSLLNARTRKNYLAWYQQKPGQPPKLLI |
| 12B-17 chain | | YWASTRESGVPDRFSGSGSGTDFTLTISSLQAEDVAVYYCKQSYILRTFGGGTK |
| #3 | | VEIKRTVAAPSVFIFPPSDEQLKSGTASVVCLLNNFYPREAKVQWKVDNALQSG |
| (VK-hck) | | NSQESVTEQDSKDSTYSLSSTLTLSKADYEKHKVYACEVTHQGLSSPVTKSENR |
| | | GEC |
| MAT1007- | 39 | PKSCDKTHTCPPCPAPEAAGGPSVFLFPPKPKDTLMISRTPEVTCVVVDVSHED |
| 12B-17 chain | | PEVKFNWYVDGVEVHNAKTKPREEQYNSTYRVVSVLTVLHQDWLNGKEYKCKVS |
| #4 | | NKALPAPIEKTISKAKGQPREPQVCTLPPSREEMTKNQVSLSCAVKGFYPSDIA |
| (FC) | | VEWESNGQPENNYKTTPPVLDSDGSFFLVSKLTVDKSRWQQGNVESCSVMHEAL |
| | | HNHYTQKSLSLSPGK |
| MAT1007- | 40 | DIQMTQSPSSLSASVGDRVTITCKASQDINKYITWYQQKPGKAPKLLIYYTSTL |
| 12B-18 chain | | QPGVPSRFSGSGSGTDYTFTISSLQPEDIATYYCLQYDSLLWTFGGGTKVEIKR |
| #1 | | TVAAPSVFIFPPSDEQLKSGTASVVCLLNNFYPREAKVQWKVDNALQSGNSQES |
| (VK-hck- | | VTEQDSKDSTYSLSSTLTLSKADYEKHKVYACEVTHQGLSSPVTKSENRGECEV |
| VH-hlgG1) | | QLVQSGAEVKKPGASVKVSCKASGESFTNYYVHWMRQAPGQGLEWIGWISPGSD |
| | | NTKYNEKFKGRVTLTADTSISTAYMELSRLRSDDTAVYYCARDDYGNYYFDYWG |
| | | QGTTVTVSSASTKGPSVFPLAPSSKSTSGGTAALGCLVKDYFPEPVTVSWNSGA |
| | | LTSGVHTFPAVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGKEYKCKVS |
| | | NKALPAPIEKTISKAKGQPREPQVYTLPPCREEMTKNQVSLWCLVKGFYPSDIA |
| | | VEWESNGQPENNYKTTPPVLDSDGSFFLYSKLTVDKSRWQQGNVFSCSVMHEAL |
| | | HNHYTQKSLSLSPGK |
| MAT1007- | 35 | EVQLVQSGAEVKKPGSSVKVSCKASGYTFSRSWMNWVRQAPGQGLEWMGRIYPG |
| 12B-18 chain | | NADIKYNANFKGRVTITADKSTSTAYMELSSLRSEDTAVYYCAHIYYDFYYALD |
| #2 | | YWGQGTTVTVSSASTKGPSVFPLAPSSKSTSGGTAALGCLVKDYFPEPVTVSWN |
| (VH-CH1) | | SGALTSGVHTFPAVLQSSGLYSLSSVVTVPSSSLGTQTYICNVNHKPSNTKVDK |
| | | KVEPKSC |
| MAT1007- | 36 | DIVMTQSPDSLAVSLGERATINCKSSQSLLNARTRKNYLAWYQQKPGQPPKLLI |
| 12B-18 chain | | YWASTRESGVPDRESGSGSGTDFTLTISSLQAEDVAVYYCKQSYILRTFGGGTK |
| #3 | | VEIKRTVAAPSVFIFPPSDEQLKSGTASVVCLLNNFYPREAKVQWKVDNALQSG |
| (VK-hck) | | NSQESVTEQDSKDSTYSLSSTLTLSKADYEKHKVYACEVTHQGLSSPVTKSENR |
| | | GEC |
| MAT1007- | 39 | PKSCDKTHTCPPCPAPEAAGGPSVELFPPKPKDTLMISRTPEVTCVVVDVSHED |
| 12B-18 chain | | PEVKENWYVDGVEVHNAKTKPREEQYNSTYRVVSVLTVLHQDWLNGKEYKCKVS |
| #4 | | NKALPAPIEKTISKAKGQPREPQVCTLPPSREEMTKNQVSLSCAVKGFYPSDIA |
| (FC) | | VEWESNGQPENNYKTTPPVLDSDGSFFLVSKLTVDKSRWQQGNVFSCSVMHEAL |
| | | HNHYTQKSLSLSPGK |

The recombinant FIT-Ig and MAT-Fab proteins were transiently expressed and purified as described herein. For each FIT-Ig or MAT-Fab, 3 or 4 plasmids respectively encoding the corresponding polypeptide chains were co-transfected into HEK 293F cells. After approximately six days of post-transfection cell culture, the supernatants were harvested and subjected to Protein A affinity chromatography. The composition and purity of the purified antibodies were analyzed by size exclusion chromatography (SEC). Purified antibody, in PBS, was applied to a TSKgel SuperSW3000, 300×4.6 mm, SEC column (TOSOH). A DIONEX™ UltiMate 3000 HPLC instrument (Thermo Scientific) was used for SEC using UV detection at 280 nm and 214 nm. The expression and SEC-HPLC results are shown in Table 13 below.

TABLE 13
Production characterization of
ROR1-mAb004 FIT-Ig and MAT-Fab

| FIT-Ig Identifier | Expression Titer | Purity % (SEC-HPLC) |
|---|---|---|
| FIT1007-12B-17 | 14.12 mg/L | 100 |
| FIT1007-12B-18 | 12.52 mg/L | 99.89 |
| MAT1007-12B-17 | 23.15 mg/L | 98.52 |
| MAT1007-12B-18 | 29.32 mg/L | 95.64 |

ROR1 binding affinity/kinetics of the humanized candidate FIT1007-12B-17 and its parental chimeric FIT-Ig FIT1007-12B were measured using the same method as described in Example 3. For each antibody, measurements were titrated by 6 antigen concentrations, i.e., 3 fold diluted from 500 nM. The binding kinetics and affinity are shown in Table 14 below. Binding kinetics of FIT1007-12B-18, MAT1007-12B-17 and MAT1007-12B-18 are similar to those of FIT1007-12B-17. These candidates share the same ROR1 binding Fab.

TABLE 14
ROR1 binding kinetics of candidate

| Sample ID | KD (M) | kon(1/Ms) | kdis(1/s) |
|---|---|---|---|
| FIT1007-12B | 5.67E−09 | 1.82E+05 | 1.03E−03 |
| FIT1007-12B-17 | 5.25E−10 | 2.24E+05 | 1.17E−04 |

### Example 6. Binding Characterization of Humanized FIT-Ig and MAT-Fab

Cell binding activity of ROR1×CD3 antibodies were measured with a human TCR/CD3 complex transfected CHO cell line (CHO-CD3-TCR) and ROR1-expressing tumor cell lines (NCI-H1975, MDA-MB-231, A549 and RPMI8226). Briefly, 5×10⁵ cells were seeded into each well of a 96-well plate. Cells were centrifuged at 400 g for 5 minutes and supernatants were discarded. For each well, 100 IA of serially diluted antibodies were then added and mixed with the cells. After 40 minutes of incubation at 4° C., plates were washed several times to remove excess antibodies. Secondary fluorochrome-conjugated goat anti-human IgG antibody was then added and incubated with cells at room temperature for 20 minutes. After another round of centrifugation and a washing step, cells were resuspended in FACS buffer for reading on a CytoFLEX Flow Cytometer (Beckman Coulter). Median Fluorescence Intensity (MFI) readouts were plotted against antibody concentration and analyzed with GraphPad Prism 6.0 software.

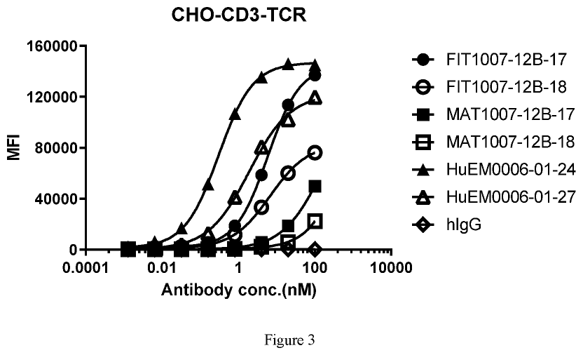

As shown in FIG. 3, CHO-CD3-TCR binding potency correlated with the CD3 binding affinity and valency of each molecule. By comparing FIT-Ig with its parental anti-CD3 monoclonal IgG1 antibody, i.e. FIT1007-12B-17 v.s. HuEM0006-01-24 (VH/VL sequences: SEQ ID NOs: 22 and 24, Table 7), or FIT1007-12B-18 v.s. HuEM0006-01-27 (VH/VL sequences: SEQ ID NOs: 23 and 24, Table 7), FIT-Ig showed relatively lower binding potency, which may be due to steric hindrance.

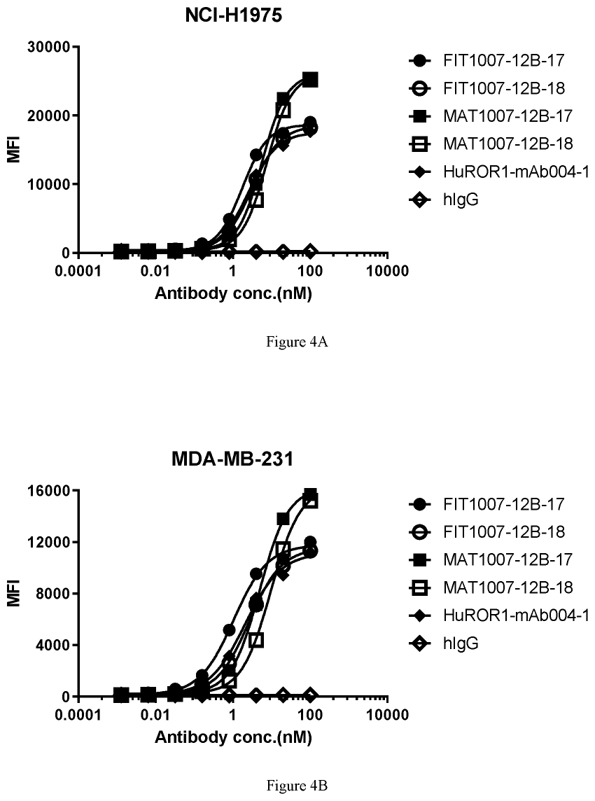

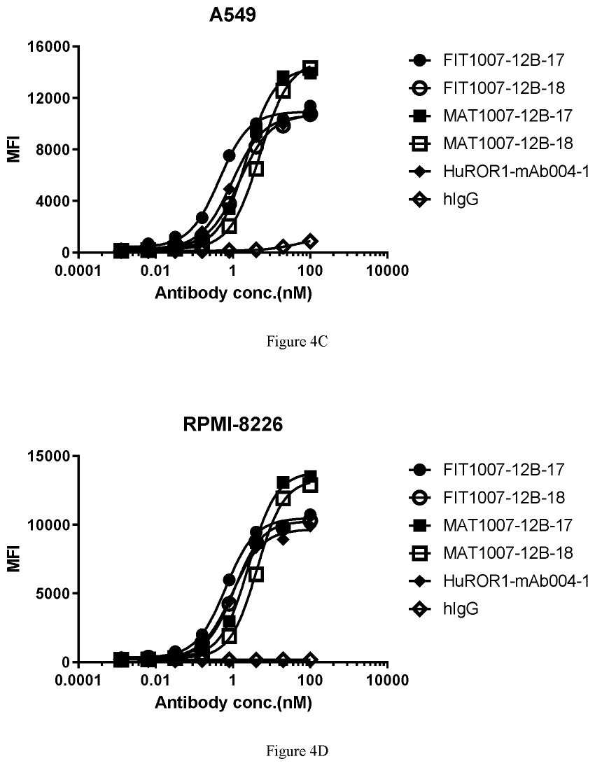

As shown in FIG. 4A-D, binding potency to ROR1-expressing tumor cells are relatively similar between FIT-Ig and their shared parental anti-ROR1 monoclonal antibody (HuROR1-mAb004-1, with the sequences of ROR1-mAb004VH.la(AA) and ROR1-mAb004VK.la, SEQ ID NOs: 21 and 13). The binding curve of MAT-Fab appear different from FIT-Ig and its parental anti-ROR1 monoclonal antibody, which may be due to the different target binding valency.

### Example 7. Redirected CD3 Activation of Humanized FIT-Ig and MAT-Fab

To measure redirected CD3 activation by ROR1×CD3 bispecific FIT-Ig and MAT-Fab antibodies, a co-cultured reporter gene assay was used. In this assay, Jurkat-NFAT-luc cells trigger downstream luciferase signal when cell surface CD3 is activated. RPMI8226 cells were used as the ROR1-expressing target cell, which can crosslink CD3/TCR complex on T cells via bispecific ROR1×CD3 antibodies upon ROR1 binding. Jurkat-NFAT-luc and RPMI8226 cells were washed and resuspended in assay medium (RPMI1640 with 10% FBS) separately. Both cell types were seeded into 96-well plates (Costar #3903) at 1×10⁵ cells per well in a ratio of 1:1. FIT-Ig or MAT-Fab antibodies were added and mixed with the cells and incubated for 4 hours at 37° C. At the end of incubation, ONE-Glo™ luminescence assay kit (Promega, Cat. #E6130) reagents were prepared and added into wells according the manufacturer's instructions. Plates were read for luminescence signals with Varioskan™ LUX microplate reader (ThermoFisher Scientific). The results are shown in FIG. 5.

One irrelevant negative control FIT-Ig, anti-EGFR×cMET bispecific molecule (EMB01) and two anti-CD3 monoclonal antibodies, namely, HuEM0006-01-24 and HuEM0006-01-27, were also tested. All of the bispecific ROR1×CD3 binding proteins led to increased T cell activation in the presence of ROR1-expressing target cells in comparison to monospecific anti-CD3 binding proteins having no ROR1 binding activity.

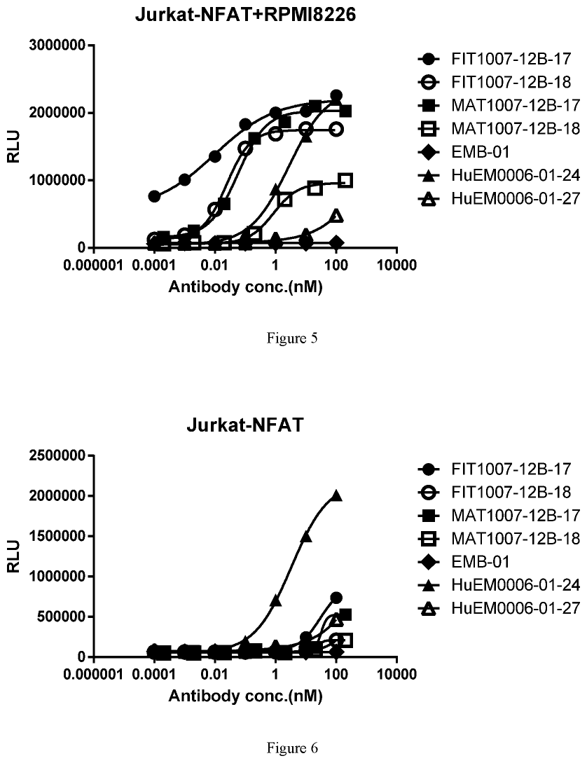

Non-target redirected CD3 activation was tested using a Jurkat-NFAT-luc based reporter gene assay in the absence of target cells. The results are shown in FIG. 6. This assay was conducted in the absence of cells expressing a co-target for the bispecific binding proteins, in this case ROR1. Bispecific ROR1×CD3 antibodies showed less non-target redirected activation than the anti-CD3 antibody alone, in the absence of ROR1-expressing target cells.

### Example 8. Redirected T Cell Cytotoxicity of Humanized FIT-Ig and MAT-Fab

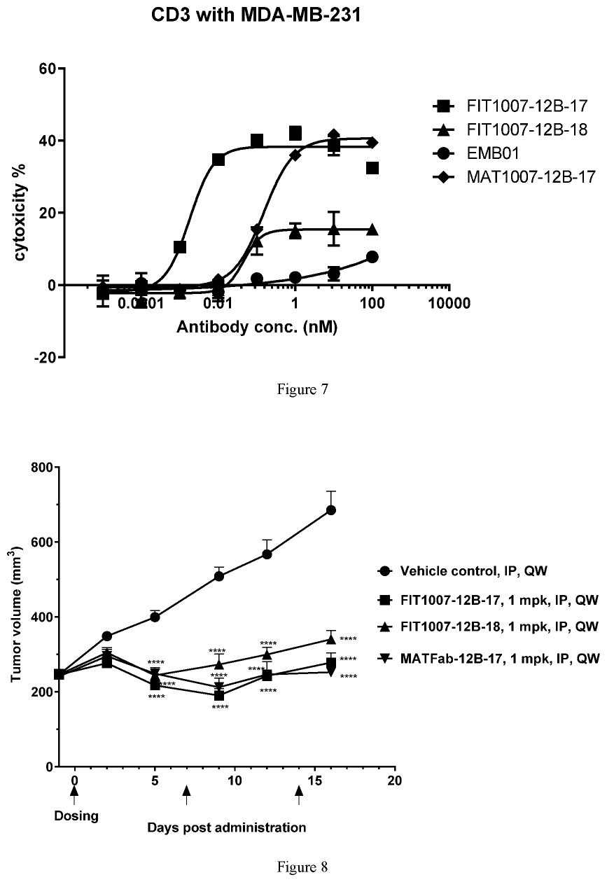

The tumor cell killing potency of ROR1×CD3 bispecific binding proteins was measured in a redirected T cell cytotoxicity assay using the human breast cancer cell line MDA-MB-231 as target cells and human T cells as effector cells. Briefly, cells were harvested, washed, and resuspended with assay medium (RPMI1640 with 10% FBS). MDA-MB-231 cells were seeded into flat-bottom 96-well plates (Corning, Cat. #3599) at 5×10⁴ cells per well. T cells were purified from human PBMC with a commercial PBMC isolation kit (EasySep™, Stemcell Technologies, Cat. #17951) and were added to the wells at 2×10⁵ cells per well. Test antibodies were added and incubated with the mixture of the cells for 48 hours at 37° C. Lactate dehydrogenase (LDH) release was measured with a CytoTox 96® cytotoxicity assay kit (Promega, Cat. #G1780). OD490 readouts were obtained following the manufacturer's instructions. The max and min lysis were also generated according the CytoTox kit (Promega, #G1780) instruction. The max lysis was generated by adding lysis buffer to samples which only have tumor cells. The min lysis was generated from the culture medium background. The min lysis was subtracted from the readouts of all samples. Target cells MDA-MB-231 max lysis (100%) minus minimal lysis (0%) was presented as the normalization denominator. The percentage of LDH release was plotted against the concentrations of bispecific antibodies. As shown in FIG. 7, ROR1×CD3 bispecific binding proteins demonstrated redirected T cell cytotoxicity to MDA-MB-231 tumor cells, while the EGFR×cMET bispecific binding FIT-Ig EMB01 showed low cytotoxic activity.

### Example 9. MDA-MB-231 Tumor Volume in Human PBMC Engrafted M-NSG Mice Treated with ROR1×CD3 Bispecific Antibodies

Antitumor efficacy was evaluated in M-NSG mice, which is an immunodeficient strain lacking T cells, B cells and natural killer cells. MDA-MB-231 cells (5×10⁶) were injected subcutaneously into the right dorsal flank. Five days after tumor cell inoculation, the mice received a single intraperitoneal dose of 3.5×10⁶ human PBMC. The animals were randomized based on tumor size (˜150-300 mm 3) on day 15 and treatment was initiated in the next day. Tumor growth was monitored by caliper measurements. The study was terminated on day 16 after the first administration, and mice were euthanized when GVHD signs appeared. Mice were treated once a week for 3 weeks (QW×3) with 1 mg/kg of FIT1007-12B-17, FIT1007-12B-18, MAT1007-12B-17 or vehicle by intraperitoneal (i.p.) injection. As shown in FIG. 8, FIT-Ig and MAT-Fab treatment group mice showed significant tumor growth inhibition by comparing with vehicle group (****P<0.0001; compared to Vehicle group, Two-way ANOVA combined with Dunnett test).

### Example 10. Internalization Characterization of Humanized Anti-ROR1 Antibodies

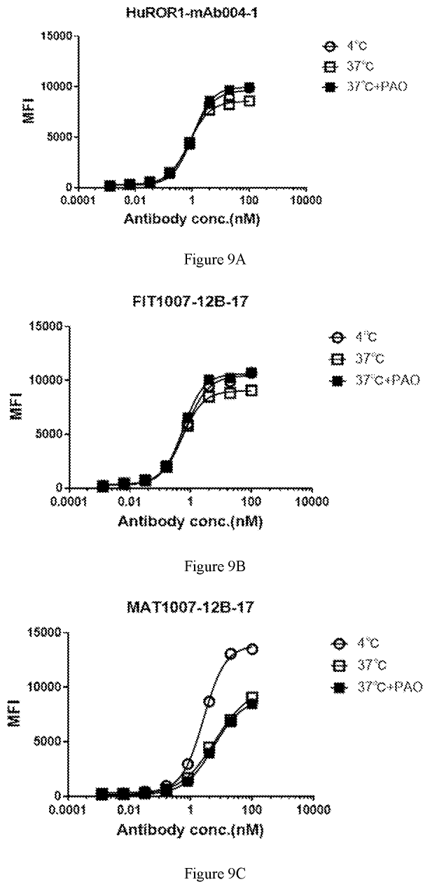

The binding internalization of humanized anti-ROR1 antibodies were characterized with ROR1-expressing myeloma cell line RPMI8226 with a method similar to that described previously in Example 1.4. Briefly, cells were harvested and resuspended in FACS buffer at density of 3 million per mL. Diluted antibodies were added to the tubes and incubated for 30 min at 4° C. After the first incubation, cells were washed three times with cold PBS to remove unbound antibody. Then, the cells of each antibody treatment were split into three groups, 4° C., 37° C. and 37° C.+PAO, respectively. Cells in the 37° C. “internalization” group were resuspended in pre-warmed medium and incubated at 37° C. for 2 hours to allow internalization, while cells in the 4° C. “control” group were kept at 4° C. for the same period. Cells in the “37° C.+PAO” group were resuspended in pre-warmed medium and incubated in the presence of 304 Phenylarsine Oxide (an endocytosis inhibitor to prevent internalization of membrane proteins), at 37° C. for 2 hours. The 37° C.+PAO treatment group served the purpose to calibrate the effect of antibody dissociation. After the second incubation, cells were washed once with cold PBS and incubated with fluorescein labeled secondary antibody for 30 min at 4° C. After another round of centrifugation and washing step, cells were resuspended in FACS buffer for reading on a CytoFLEX Flow Cytometer (Beckman Coulter). An irrelevant mouse IgG control (MFI_{background}) was calculated and used for background calibration. The difference between the MFI readout of “control” and that of “internalization” (ΔMFI) reflects the internalization of ROR1 antibodies, and such difference in relative to calibrated MFI of “control” reflects percentage of antibody internalization, which is calculated as follows and is summarized below in Table 15. As shown in FIG. 9, at 100 nM antibody concentration, HuROR1-mAb004-1 and its respective FIT-Ig/MAT-Fab showed limited internalization. The calculated antibody internalization percentages of HuROR-mAb004-1 and FIT1007-12B-17 were consistent with the results shown in Example 1.4, Table 4.

MAT-Fab showed reduced binding at 37° C., which may be due to its lower binding valency and higher binding off-rate at 37° C. For the calculation of MAT-Fab internalization, the binding curve did not reach the binding plateau at 100 nM.

Percentage of internalization (ΔMFI)=[1−(MFI_{internalization}−MFI_{background})/(MFI_{control}−MFI_{background})]×100%.

TABLE 15
MFI reduction and calibrated internalization
percentage of humanized anti-ROR1 antibodies

| | MFI reduction (Internalization |
|---|---|
| Sample ID | percentage after calibration*) |
| HuROR1-mAb004-1 | 13% (13%) |
| FIT1007-12B-17 | 15% (15%) |
| MAT1007-12B-17 | 33% (−4%) |

*The number in brackets was calibrated with the number of PAO treatment group

### Example 11. Ref FIT-Ig Generation and In Vitro Activity Comparison with FIT1007-12B-17

The anti-CD3 antibody sequences shown in Table 7 were used to generate FIT-Igs, with the VH/VL sequences of one of the two reference anti-ROR1 antibodies, ROR1-Tab1 (clone R12) and ROR1-Tab2 (clone D10). The construction and generation of the reference FIT-Igs were performed as described in Example 3. No linkers between the immunoglobulin domains were used. The complete sequences for the FIT-Ig binding proteins are provided in the sequence information in Tables 16 and 17. Cell surface binding activity of reference FIT-Igs was assessed by using the method as described in Example 1.3, and the redirected cytotoxicity activity was assessed by using the method as described in Example 6.

The VH/VL sequences of one of the two reference anti-ROR1 antibodies, ROR1-Tab1 (clone R12) and ROR1-Tab2 (clone D10), used in this Example are as follows:

VH sequnece of antibody D10
(SEQ ID NO: 42)
QVQLKESGPGLVAPSQTLSITCTVSGFSLTSYGVHWVRQPPGKGLEWLG
VIWAGGFTNYNSALKSRLSISKDNSKSQVLLKMTSLQTDDTAMYYCARR
GSSYSMDYWGQGTSVTVSS
VL sequence of antibody D10
(SEQ ID NO: 43)
EIVLSQSPAITAASLGQKVTITCSASSNVSYIHWYQQRSGTSPRPWIYE
ISKLASGVPVRESGSGSGTSYSLTISSMEAEDAAIYYCQQWNYPLITFG
SGTKLEIQ
VH sequnece of antibody R12
(SEQ ID NO: 44)
QEQLVESGGRLVTPGGSLTLSCKASGEDESAYYMSWVRQAPGKGLEWIA
TIYPSSGKTYYATWVNGRFTISSDNAQNTVDLQMNSLTAADRATYFCAR
DSYADDGALENIWGPGTLVTISS
VL sequence of antibody R12
(SEQ ID NO: 45)
ELVLTQSPSVSAALGSPAKITCTLSSAHKTDTIDWYQQLQGEAPRYLMQ
VQSDGSYTKRPGVPDRESGSSSGADRYLIIPSVQADDEADYYCGADYIG
GYVFGGGTQLTVTG

TABLE 16
Amino acid sequences of reference FIT-Ig component chains

| Polypeptide | SEQ ID NO. | Amino acid sequence |
|---|---|---|
| D10 x CD3 | 46 | EIVLSQSPAITAASLGQKVTITCSASSNVSYIHWYQQRSGTSPRP |
| FIT-Ig Chain | | WIYEISKLASGVPVRESGSGSGTSYSLTISSMEAEDAAIYYCQQW |
| #1 | | NYPLITFGSGTKLEIQRTVAAPSVFIFPPSDEQLKSGTASVVCLL |
| | | NNFYPREAKVQWKVDNALQSGNSQESVTEQDSKDSTYSLSSTLTL |
| | | SKADYEKHKVYACEVTHQGLSSPVTKSENRGECEVQLVQSGAEVK |
| | | KPGASVKVSCKASGFSFTNYYVHWMRQAPGQGLEWMGWISPGSDN |
| | | TKYNEKFKGRVTMTRDTSISTAYMELSRLRSDDTAVYYCARDDYG |
| | | NYYFDYWGQGTTVTVSSASTKGPSVFPLAPSSKSTSGGTAALGCL |
| | | VKDYFPEPVTVSWNSGALTSGVHTFPAVLQSSGLYSLSSVVTVPS |
| | | SSLGTQTYICNVNHKPSNTKVDKKVEPKSCDKTHTCPPCPAPEAA |
| | | GGPSVFLFPPKPKDTLMISRTPEVTCVVVDVSHEDPEVKENWYVD |
| | | GVEVHNAKTKPREEQYNSTYRVVSVLTVLHQDWLNGKEYKCKVSN |
| | | KALPAPIEKTISKAKGQPREPQVYTLPPSREEMTKNQVSLTCLVK |
| | | GFYPSDIAVEWESNGQPENNYKTTPPVLDSDGSFFLYSKLTVDKS |
| | | RWQQGNVFSCSVMHEALHNHYTQKSLSLSPGK |
| D10 x CD3 | 47 | QVQLKESGPGLVAPSQTLSITCTVSGFSLTSYGVHWVRQPPGKGL |
| FIT-Ig Chain | | EWLGVIWAGGFTNYNSALKSRLSISKDNSKSQVLLKMTSLQTDDT |
| #2 | | AMYYCARRGSSYSMDYWGQGTSVTVSSASTKGPSVFPLAPSSKST |
| | | SGGTAALGCLVKDYFPEPVTVSWNSGALTSGVHTFPAVLQSSGLY |
| | | SLSSVVTVPSSSLGTQTYICNVNHKPSNTKVDKKVEPKSC |
| D10 x CD3 | 48 | DIVMTQSPDSLAVSLGERATINCKSSQSLLNARTRKNYLAWYQQK |
| FIT-Ig Chain | | PGQPPKLLIYWASTRESGVPDRFSGSGSGTDFTLTISSLQAEDVA |
| #3 | | VYYCKQSYILRTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSENRGEC |
| R12 x CD3 | 49 | ELVLTQSPSVSAALGSPAKITCTLSSAHKTDTIDWYQQLQGEAPR |
| FIT-Ig Chain | | YLMQVQSDGSYTKRPGVPDRFSGSSSGADRYLIIPSVQADDEADY |
| #1 | | YCGADYIGGYVFGGGTQLTVTGGQPKAAPSVTLFPPSSEELQANK |
| | | ATLVCLISDFYPGAVTVAWKADSSPVKAGVETTTPSKQSNNKYAA |
| | | SSYLSLTPEQWKSHRSYSCQVTHEGSTVEKTVAPTECSEVQLVQS |
| | | GAEVKKPGASVKVSCKASGFSFTNYYVHWMRQAPGQGLEWMGWIS |
| | | PGSDNTKYNEKFKGRVTMTRDTSISTAYMELSRLRSDDTAVYYCA |
| | | RDDYGNYYFDYWGQGTTVTVSSASTKGPSVFPLAPSSKSTSGGTA |
| | | ALGCLVKDYFPEPVTVSWNSGALTSGVHTFPAVLQSSGLYSLSSV |
| | | VTVPSSSLGTQTYICNVNHKPSNTKVDKKVEPKSCDKTHTCPPCP |
| | | APEAAGGPSVFLFPPKPKDTLMISRTPEVTCVVVDVSHEDPEVKE |
| | | NWYVDGVEVHNAKTKPREEQYNSTYRVVSVLTVLHQDWLNGKEYK |
| | | CKVSNKALPAPIEKTISKAKGQPREPQVYTLPPSREEMTKNQVSL |
| | | TCLVKGFYPSDIAVEWESNGQPENNYKTTPPVLDSDGSFFLYSKL |
| | | TVDKSRWQQGNVFSCSVMHEALHNHYTQKSLSLSPGK |
| R12 x CD3 | 50 | QEQLVESGGRLVTPGGSLTLSCKASGEDESAYYMSWVRQAPGKGL |
| FIT-Ig Chain | | EWIATIYPSSGKTYYATWVNGRFTISSDNAQNTVDLQMNSLTAAD |
| #2 | | RATYFCARDSYADDGALFNIWGPGTLVTISSASTKGPSVFPLAPS |
| | | SKSTSGGTAALGCLVKDYFPEPVTVSWNSGALTSGVHTEPAVLQS |
| | | SGLYSLSSVVTVPSSSLGTQTYICNVNHKPSNTKVDKKVEPKSC |
| R12 x CD3 | 51 | DIVMTQSPDSLAVSLGERATINCKSSQSLLNARTRKNYLAWYQQK |
| FIT-Ig Chain | | PGQPPKLLIYWASTRESGVPDRESGSGSGTDFTLTISSLQAEDVA |
| #3 | | VYYCKQSYILRTFGGGTKVEIKRTVAAPSVFIFPPSDEQLKSGTA |
| | | SVVCLLNNFYPREAKVQWKVDNALQSGNSQESVTEQDSKDSTYSL |
| | | SSTLTLSKADYEKHKVYACEVTHQGLSSPVTKSENRGEC |

FIG. 11 demonstrates comparison of FIT1007-12B-17 to the reference FIT-Ig molecules provided in Table 16. FIGS. 11A and 11B show FIT1007-12B-17 and the reference FIT-Igs exhibited similar cell surface binding to both ROR1 expressing MDA-MB-231 and CD3 expressing Jurkat cells. However, as shown in FIG. 11C on redirected T cell cytotoxicity against MDA-MB-231 cells, FIT1007-12B-17 achieved more potent cytotoxicity than the reference FIT-Ig molecules did.

1. An isolated antibody or antigen-binding fragment thereof that specifically binds to ROR1, comprising a set of six CDRs, CDR-H1, CDR-H2, CDR-H3, CDR-L1, CDR-L2, and CDR-L3, wherein:
the CDR-H1 comprises the amino acid sequence of RSWMN (SEQ ID NO:1);
the CDR-H2 comprises the sequence of RIYPGNGDIKYNGNFKG (SEQ ID NO: 2) or RIYPGNADIKYNANFKG (SEQ ID NO: 4);
the CDR-H3 comprises the sequence of IYYDFYYALDY (SEQ ID NO: 3);
the CDR-L1 comprises the sequence of KASQDINKYIT (SEQ ID NO: 5);
the CDR-L2 comprises the sequence of YTSTLQP (SEQ ID NO: 6); and
the CDR-L3 comprises the sequence of LQYDSLLWT (SEQ ID NO: 7),
optionally wherein the CDRs are defined according to Kabat numbering.

2. The isolated antibody or antigen-binding fragment of claim 1, wherein the antibody comprises a variable heavy chain domain VH and a variable light chain domain VL, wherein:
the VH domain comprises the sequence of SEQ ID NO:8 or 17, or a sequence having at least 80%, 85%, 90%, 91%, 92%, 93%, 94%, 95%, 96%, 97%, 98%, 99% or more identity therewith, and/or the VL domain comprises the sequence of SEQ ID NO:9, or a sequence having at least 80%, 85%, 90%, 91%, 92%, 93%, 94%, 95%, 96%, 97%, 98%, 99% or more identity therewith;
or
the VH domain comprises the sequence selected from any one of SEQ ID NOs: 10-12 and 21, or a sequence having at least 80%, 85%, 90%, 91%, 92%, 93%, 94%, 95%, 96%, 97%, 98%, 99% or more identity therewith, and/or the VL domain comprises the sequence selected from any one of SEQ ID NOs: 13-16, or a sequence having at least 80%, 85%, 90%, 91%, 92%, 93%, 94%, 95%, 96%, 97%, 98%, 99% or more identity therewith.

3. The isolated antibody or antigen-binding fragment of claim 1, wherein the antibody is a chimeric or humanized antibody, optionally the antibody is a humanized antibody,
and further optionally, the VH domain of the antibody comprises amino acid residues 1E, 27Y, and 94H, and 0 to 4 residues selected from 38K, 481, 66K, and 67A, according to Kabat numbering; and the VL domain comprises amino acid residue 71Y, and 0 to 4 residues selected from 4L, 49H, 581, and 69R, according to Kabat numbering.

4. The isolated antibody or antigen-binding fragment of claim 1, wherein the antibody comprises a combination of VH and VL sequences selected from the group consisting of:

| combination | VH sequence | VL sequence |
|---|---|---|
| 1 | SEQ ID NO: 8 | SEQ ID NO: 9 |
| 2 | SEQ ID NO: 17 | SEQ ID NO: 9 |
| 3 | SEQ ID NO: 10 | SEQ ID NO: 13 |
| 4 | SEQ ID NO: 10 | SEQ ID NO: 14 |
| 5 | SEQ ID NO: 10 | SEQ ID NO: 15 |
| 6 | SEQ ID NO: 10 | SEQ ID NO: 16 |
| 7 | SEQ ID NO: 11 | SEQ ID NO: 13 |
| 8 | SEQ ID NO: 11 | SEQ ID NO: 14 |
| 9 | SEQ ID NO: 11 | SEQ ID NO: 15 |
| 10 | SEQ ID NO: 11 | SEQ ID NO: 16 |
| 11 | SEQ ID NO: 12 | SEQ ID NO: 13 |
| 12 | SEQ ID NO: 12 | SEQ ID NO: 14 |
| 13 | SEQ ID NO: 12 | SEQ ID NO: 15 |
| 14 | SEQ ID NO: 12 | SEQ ID NO: 16 |
| 15 | SEQ ID NO: 21 | SEQ ID NO: 13 |
| 16 | SEQ ID NO: 21 | SEQ ID NO: 14 |
| 17 | SEQ ID NO: 21 | SEQ ID NO: 15 |
| 18 | SEQ ID NO: 21 | SEQ ID NO: 16 |

optionally, the antibody comprises a VH domain comprising the sequence of SEQ ID NO: 21 and a VL domain comprising the sequence of SEQ ID NO: 13.

5. The isolated antibody or antigen-binding fragment of claim 1, wherein the antibody has one or more of the following characteristics:
(i) upon binding to the cell surface of ROR1-expressing cells (e.g. ROR1-expressing myeloma cell line), the antibody is internalized not more than 20%, optionally not more than 15%, or 14%, 13%, 12%, 11%, as measured in a cell based assay, wherein the internalization can be reflected by a decrease percentage in the median fluorescence intensity (MFI), as detected by flow cytometry, of the antibody binding to the surface of ROR1-expressing cells (e.g. ROR1-expressing myeloma cell line) after a two-hour incubation at 37° C., relative to a control kept at 4° C. for the same period;
(ii) the antibody binds to human ROR1 at C-terminus of the ROR1's Ig-like domain, and optionally competes with an antibody with a VH/VL sequence pair of SEQ ID NOs: 42 and 43 for binding to ROR1;
(iii) binding of the antibody to ROR1 induces anti-tumor activity, e.g., reduced tumor burden/growth/cell expansion.

6. A fusion or a conjugate comprising the isolated antibody or antigen-binding fragment of claim 1.

7. A nucleic acid molecule encoding the isolated antibody or antigen-binding fragment of claim 1.

8. A vector comprising the nucleic acid molecule of claim 7.

9. A host cell expressing the nucleic acid molecule encoding the isolated antibody or antigen-binding fragment of claim 1.

10. A pharmaceutical composition comprising the isolated antibody or antigen-binding fragment of claim 1.

11. A method of detecting ROR1 in a biological sample, comprising contacting the biological sample with the isolated antibody or antigen-binding fragment of claim 1.

12. A bispecific binding protein that specifically binds ROR1 and CD3, comprising:
a) a first antigen-binding site that specifically binds ROR1; and
b) a second antigen-binding site that specifically binds CD3,
wherein the first antigen-binding site comprises a set of six CDRs, CDR-H1, CDR-H2, CDR-H3, CDR-L1, CDR-L2, and CDR-L3, wherein:
CDR-H1 comprises the sequence of RSWMN (SEQ ID NO:1),
CDR-H2 comprises the sequence of RIYPGNGDIKYNGNFKG (SEQ ID NO: 2) or RIYPGNADIKYNANFKG (SEQ ID NO: 4),
CDR-H3 comprises the sequence of IYYDFYYALDY (SEQ ID NO: 3),
CDR-L1 comprises the sequence of KASQDINKYIT (SEQ ID NO: 5),
CDR-L2 comprises the sequence of YTSTLQP (SEQ ID NO: 6), and
CDR-L3 comprises the sequence of LQYDSLLWT (SEQ ID NO: 7),
wherein the CDRs are defined according to Kabat numbering,
optionally, the first antigen-binding site comprises a VH domain and a VL domain as defined in claim 2.

13. The bispecific binding protein of claim 12, wherein the second antigen-binding site comprises a set of six CDRs, CDR-H1, CDR-H2, CDR-H3, CDR-L1, CDR-L2, and CDR-L3, wherein:
CDR-H1 comprises the sequence of NYYVH (SEQ ID NO:25);
CDR-H2 comprises the sequence of WISPGSDNTKYNEKFKG (SEQ ID NO: 26);
CDR-H3 comprises the sequence of DDYGNYYFDY (SEQ ID NO: 27);
CDR-L1 comprises the sequence of KSSQSLLNARTRKNYLA (SEQ ID NO: 28);
CDR-L2 comprises the sequence of WASTRES (SEQ ID NO: 29); and
CDR-L3 comprises the sequence of KQSYILRT (SEQ ID NO: 30),
wherein the CDRs are defined according to Kabat numbering,
optionally, the second antigen-binding site comprises:
a VH domain comprising the sequence of SEQ ID NO: 22 or 23, or a sequence having at least 80%, 85%, 90%, 91%, 92%, 93%, 94%, 95%, 96%, 97%, 98%, 99% or more identity therewith, and/or
a VL domain comprising the sequence of SEQ ID NO: 24, or a sequence having at least 80%, 85%, 90%, 91%, 92%, 93%, 94%, 95%, 96%, 97%, 98%, 99% or more identity therewith.

14. The bispecific binding protein of claim 12, comprising a first polypeptide chain, a second polypeptide chain and a third polypeptide chain,
wherein
(i) the first polypeptide chain comprises, from amino terminus to carboxyl terminus, VL_{A}-CL-VH_{B}-CH1-Fc wherein CL is fused directly to VH_{B}; the second polypeptide chain comprises, from amino to carboxyl terminus, VH_{A}-CH1; the third polypeptide chain comprises, from amino to carboxyl terminus, VL_{B}-CL; or
(ii) the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1;
wherein VL is a light chain variable domain, CL is a light chain constant domain, VH is a heavy chain variable domain, CH1 is a heavy chain constant domain, Fc is an immunoglobulin Fc region, for example, the Fc of IgG1 (optionally, comprising, from amino terminus to carboxyl terminus, hinge-CH2-CH3),
wherein the VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A is ROR1, and the second antigen B is CD3,
wherein two of the first polypeptide chains, two of the second polypeptide chains, and two of the third polypeptide chains are associated to form a FIT-Ig protein.

15. The bispecific binding protein of claim 14, wherein:
the first polypeptide chain comprises an amino acid sequence of SEQ ID NO:34 or 37, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith, and
the third polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith.

16. The bispecific binding protein of claim 12, comprising a first polypeptide chain, a second polypeptide chain, a third polypeptide chain, and a fourth polypeptide chain,
wherein
(i) the first polypeptide chain comprises, from amino terminus to carboxyl terminus, VL_{A}-CL-VH_{B}-CH1-Fc wherein CL is fused directly to VH_{B}; the second polypeptide chain comprises, from amino to carboxyl terminus, VH_{A}-CH1; the third polypeptide chain comprises, from amino to carboxyl terminus, VL_{B}-CL; the fourth polypeptide chain comprises Fc; or
(ii) the first polypeptide chain comprises, from amino terminus to carboxyl terminus, VH_{A}-CH1-VL_{B}-CL-Fc wherein CH1 is fused directly to VL_{B}; the second polypeptide chain comprises, from amino to carboxyl terminus, VL_{A}-CL; the third polypeptide chain comprises, from amino to carboxyl terminus, VH_{B}-CH1; the fourth polypeptide chain comprises Fc;
wherein VL is a light chain variable domain, CL is a light chain constant domain, VH is a heavy chain variable domain, CH1 is a heavy chain constant domain, Fc is an immunoglobulin Fc region (optionally, comprising, from amino terminus to carboxyl terminus, hinge-CH2-CH3),
wherein the VL_{A}-CL pairs with VH_{A}-CH1 to form a first Fab that specifically binds a first antigen A, and VL_{B}-CL pairs with VH_{B}-CH1 to form a second Fab that specifically binds a second antigen B, and
wherein the first antigen A is ROR1, and the second antigen B is CD3,
wherein the first polypeptide chain, the second polypeptide chain, the third polypeptide chain and the fourth polypeptide chain are associated to form a MAT-Fab protein,
optionally wherein the Fc of the first polypeptide chain and the Fc of the fourth polypeptide chain comprises heterodimerizing modifications, especially in CH3 domain, which favor heterodimerization over homodimerization of the two chains,
further optionally, the first polypeptide chain has a human IgG1 Fc region with mutation T366W as a “knob”, and the fourth polypeptide chain has a human IgG1 Fc region with mutations T366S, L368A, and Y407V as a “hole”; and/or the first polypeptide chain has a human IgG1 Fc region with S354C and the fourth polypeptide chain has a human IgG1 Fc region with mutation Y349C to form an additional disulfide bridge in the CH3 domain.

17. The bispecific binding protein of claim 16, wherein:
the first polypeptide chain comprises an amino acid sequence of SEQ ID NO:38 or 40, or a sequence having at least 80%, 85%, 90%, 91%, 92%, 93%, 94%, 95%, 96%, 97%, 98%, 99% or more identity therewith,
the second polypeptide chain comprises an amino acid sequence of SEQ ID NO:35, or a sequence having at least 80%, 85%, 90%, 91%, 92%, 93%, 94%, 95%, 96%, 97%, 98%, 99% or more identity therewith,
the third polypeptide chain comprises an amino acid sequence of SEQ ID NO:36, or a sequence having at least 80%, 85%, 90%, 91%, 92%, 93%, 94%, 95%, 96%, 97%, 98%, 99% or more identity therewith; and
the fourth polypeptide chain comprises an amino acid sequence of SEQ ID NO:39, or a sequence having at least 80%, 85%, 90%, 91%, 92%, 93%, 94%, 95%, 96%, 97%, 98%, 99% or more identity therewith.

18. A nucleic acid molecule encoding the bispecific binding protein of claim 12.

19. (canceled)

20. A host cell comprising the nucleic acid molecule of claim 18.

21. A method of preparing the isolated antibody or antigen-binding fragment claim 1, comprising:
culturing a host cell comprising a nucleic acid encoding the antibody or antigen-binding fragment under conditions that allow the production of the antibody or antigen-binding fragment; and
recovering the antibody or antigen-binding fragment from the culture.

22. (canceled)

23. A method of treating a disorder wherein ROR1 activity is detrimental, comprising administering to a subject in need thereof a therapeutically effective amount of the pharmaceutical composition of claim 10.

24-25. (canceled)


SEQUENCE LISTING (51 sequences)

SEQ ID NO: 1 - protein, 5 aa, Artificial Sequence
  Synthetic
RSWMN

SEQ ID NO: 2 - protein, 17 aa, Artificial Sequence
  Synthetic
RIYPGNGDIKYNGNFKG

SEQ ID NO: 3 - protein, 11 aa, Artificial Sequence
  Synthetic
IYYDFYYALDY

SEQ ID NO: 4 - protein, 17 aa, Artificial Sequence
  Synthetic
RIYPGNADIKYNANFKG

SEQ ID NO: 5 - protein, 11 aa, Artificial Sequence
  Synthetic
KASQDINKYIT

SEQ ID NO: 6 - protein, 7 aa, Artificial Sequence
  Synthetic
YTSTLQP

SEQ ID NO: 7 - protein, 9 aa, Artificial Sequence
  Synthetic
LQYDSLLWT

SEQ ID NO: 8 - protein, 120 aa, Artificial Sequence
  Synthetic
QVQLQQSGPELVKPGASVKISCKASGYAFSRSWMNWVKQRPEKGLEWIGRIYPGNGDIKYNGNFKGKATLTADKSSSTAYMQLSSLTSEDSAVYFCAHIYYDFYYALDYWGQGTSVTVSS

SEQ ID NO: 9 - protein, 107 aa, Artificial Sequence
  Synthetic
DIQLTQSPSSLSASLGGKVTITCKASQDINKYITWYQHKPGKGPRLLIHYTSTLQPGIPSRFSGSGSGRDYSFSISNLEPEDIATYYCLQYDSLLWTFGGGTKLEIK

SEQ ID NO: 10 - protein, 120 aa, Artificial Sequence
  Synthetic
EVQLVQSGAEVKKPGSSVKVSCKASGYTFSRSWMNWVRQAPGQGLEWMGRIYPGNGDIKYNGNFKGRVTITADKSTSTAYMELSSLRSEDTAVYYCAHIYYDFYYALDYWGQGTTVTVSS

SEQ ID NO: 11 - protein, 120 aa, Artificial Sequence
  Synthetic
EVQLVQSGAEVKKPGSSVKVSCKASGYTFSRSWMNWVRQAPGQGLEWIGRIYPGNGDIKYNGNFKGRATITADKSTSTAYMELSSLRSEDTAVYYCAHIYYDFYYALDYWGQGTTVTVSS

SEQ ID NO: 12 - protein, 120 aa, Artificial Sequence
  Synthetic
EVQLVQSGAEVKKPGSSVKVSCKASGYTFSRSWMNWVKQAPGQGLEWIGRIYPGNGDIKYNGNFKGKATITADKSTSTAYMELSSLRSEDTAVYYCAHIYYDFYYALDYWGQGTTVTVSS

SEQ ID NO: 13 - protein, 107 aa, Artificial Sequence
  Synthetic
DIQMTQSPSSLSASVGDRVTITCKASQDINKYITWYQQKPGKAPKLLIYYTSTLQPGVPSRFSGSGSGTDYTFTISSLQPEDIATYYCLQYDSLLWTFGGGTKVEIK

SEQ ID NO: 14 - protein, 107 aa, Artificial Sequence
  Synethetic
DIQMTQSPSSLSASVGDRVTITCKASQDINKYITWYQQKPGKAPKLLIHYTSTLQPGVPSRFSGSGSGRDYTFTISSLQPEDIATYYCLQYDSLLWTFGGGTKVEIK

SEQ ID NO: 15 - protein, 107 aa, Artificial Sequence
  Synthetic
DIQLTQSPSSLSASVGDRVTITCKASQDINKYITWYQQKPGKAPKLLIYYTSTLQPGVPSRFSGSGSGRDYTFTISSLQPEDIATYYCLQYDSLLWTFGGGTKVEIK

SEQ ID NO: 16 - protein, 107 aa, Artificial Sequence
  Synethetic
DIQLTQSPSSLSASVGDRVTITCKASQDINKYITWYQQKPGKAPKLLIHYTSTLQPGIPSRFSGSGSGRDYTFTISSLQPEDIATYYCLQYDSLLWTFGGGTKVEIK

SEQ ID NO: 17 - protein, 120 aa, Artificial Sequence
  Synthetic
QVQLQQSGPELVKPGASVKISCKASGYAFSRSWMNWVKQRPEKGLEWIGRIYPGNADIKYNANFKGKATLTADKSSSTAYMQLSSLTSEDSAVYFCAHIYYDFYYALDYWGQGTSVTVSS

SEQ ID NO: 18 - protein, 120 aa, Artificial Sequence
  Synthetic
QVQLQQSGPELVKPGASVKISCKASGYAFSRSWMNWVKQRPEKGLEWIGRIYPGQGDIKYQGNFKGKATLTADKSSSTAYMQLSSLTSEDSAVYFCAHIYYDFYYALDYWGQGTSVTVSS

SEQ ID NO: 19 - protein, 120 aa, Artificial Sequence
  Synthetic
QVQLQQSGPELVKPGASVKISCKASGYAFSRSWMNWVKQRPEKGLEWIGRIYPGNADIKYQGNFKGKATLTADKSSSTAYMQLSSLTSEDSAVYFCAHIYYDFYYALDYWGQGTSVTVSS

SEQ ID NO: 20 - protein, 120 aa, Artificial Sequence
  Synthetic
QVQLQQSGPELVKPGASVKISCKASGYAFSRSWMNWVKQRPEKGLEWIGRIYPGQGDIKYNANFKGKATLTADKSSSTAYMQLSSLTSEDSAVYFCAHIYYDFYYALDYWGQGTSVTVSS

SEQ ID NO: 21 - protein, 120 aa, Artificial Sequence
  Synthetic
EVQLVQSGAEVKKPGSSVKVSCKASGYTFSRSWMNWVRQAPGQGLEWMGRIYPGNADIKYNANFKGRVTITADKSTSTAYMELSSLRSEDTAVYYCAHIYYDFYYALDYWGQGTTVTVSS

SEQ ID NO: 22 - protein, 119 aa, Artificial Sequence
  Synthetic
EVQLVQSGAEVKKPGASVKVSCKASGFSFTNYYVHWMRQAPGQGLEWMGWISPGSDNTKYNEKFKGRVTMTRDTSISTAYMELSRLRSDDTAVYYCARDDYGNYYFDYWGQGTTVTVSS

SEQ ID NO: 23 - protein, 119 aa, Artificial Sequence
  Synthetic
EVQLVQSGAEVKKPGASVKVSCKASGFSFTNYYVHWMRQAPGQGLEWIGWISPGSDNTKYNEKFKGRVTLTADTSISTAYMELSRLRSDDTAVYYCARDDYGNYYFDYWGQGTTVTVSS

SEQ ID NO: 24 - protein, 112 aa, Artificial Sequence
  Synthetic
DIVMTQSPDSLAVSLGERATINCKSSQSLLNARTRKNYLAWYQQKPGQPPKLLIYWASTRESGVPDRFSGSGSGTDFTLTISSLQAEDVAVYYCKQSYILRTFGGGTKVEIK

SEQ ID NO: 25 - protein, 5 aa, Artificial Sequence
  Synthetic
NYYVH

SEQ ID NO: 26 - protein, 17 aa, Artificial Sequence
  Synthetic
WISPGSDNTKYNEKFKG

SEQ ID NO: 27 - protein, 10 aa, Artificial Sequence
  Synthetic
DDYGNYYFDY

SEQ ID NO: 28 - protein, 17 aa, Artificial Sequence
  Synthetic
KSSQSLLNARTRKNYLA

SEQ ID NO: 29 - protein, 7 aa, Artificial Sequence
  Synthetic
WASTRES

SEQ ID NO: 30 - protein, 8 aa, Artificial Sequence
  Synthetic
KQSYILRT

SEQ ID NO: 31 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 107 aa, Artificial Sequence [sequence omitted]

SEQ ID NO: 33 - protein, 103 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 34 - protein, 663 aa, Artificial Sequence
  Synthetic
DIQMTQSPSSLSASVGDRVTITCKASQDINKYITWYQQKPGKAPKLLIYYTSTLQPGVPSRFSGSGSGTDYTFTISSLQPEDIATYYCLQYDSLLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGAEVKKPGASVKVSCKASGFSFTNYYVHWMRQAPGQGLEWMGWISPGSDNTKYNEKFKGRVTMTRDTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 223 aa, Artificial Sequence
  Synthetic
EVQLVQSGAEVKKPGSSVKVSCKASGYTFSRSWMNWVRQAPGQGLEWMGRIYPGNADIKYNANFKGRVTITADKSTSTAYMELSSLRSEDTAVYYCAHIYYDFYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 219 aa, Artificial Sequence
  Synthetic
DIVMTQSPDSLAVSLGERATINCKSSQSLLNARTRKNYLAWYQQKPGQPPKLLIYWASTRESGVPDRFSGSGSGTDFTLTISSLQAEDVAVYYCKQSYILRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 663 aa, Artificial Sequence
  Synthetic
DIQMTQSPSSLSASVGDRVTITCKASQDINKYITWYQQKPGKAPKLLIYYTSTLQPGVPSRFSGSGSGTDYTFTISSLQPEDIATYYCLQYDSLLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGAEVKKPGASVKVSCKASGFSFTNYYVHWMRQAPGQGLEWIGWISPGSDNTKYNEKFKGRVTLTADTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 663 aa, Artificial Sequence
  Synthetic
DIQMTQSPSSLSASVGDRVTITCKASQDINKYITWYQQKPGKAPKLLIYYTSTLQPGVPSRFSGSGSGTDYTFTISSLQPEDIATYYCLQYDSLLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGAEVKKPGASVKVSCKASGFSFTNYYVHWMRQAPGQGLEWMGWISPGSDNTKYNEKFKGRVTMTRDTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 231 aa, Artificial Sequence
  Synthetic
PKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 40 - protein, 663 aa, Artificial Sequence
  Synthetic
DIQMTQSPSSLSASVGDRVTITCKASQDINKYITWYQQKPGKAPKLLIYYTSTLQPGVPSRFSGSGSGTDYTFTISSLQPEDIATYYCLQYDSLLWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGAEVKKPGASVKVSCKASGFSFTNYYVHWMRQAPGQGLEWIGWISPGSDNTKYNEKFKGRVTLTADTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 377 aa, Artificial Sequence
  Synthetic
QETELSVSAELVPTSSWNISSELNKDSYLTLDEPMNNITTSLGQTAELHCKVSGNPPPTIRWFKNDAPVVQEPRRLSFRSTIYGSRLRIRNLDTTDTGYFQCVATNGKEVVSSTGVLFVKFGPPPTASPGYSDEYEEDGFCQPYRGIACARFIGNRTVYMESLHMQGEIENQITAAFTMIGTSSHLSDKCSQFAIPSLCHYAFPYCDETSSVPKPRDLCRDECEILENVLCQTEYIFARSNPMILMRLKLPNCEDLPQPESPEAANCIRIGIPMADPINKNHKCYNSTGVDYRGTVSVTKSGRQCQPWNSQYPHTHTFTALRFPELNGGHSYCRNPGNQKEAPWCFTLDENFKSDLCDIPACDSKDSKEKNKMEILY

SEQ ID NO: 42 - protein, 117 aa, Artificial Sequence
  Synthetic
QVQLKESGPGLVAPSQTLSITCTVSGFSLTSYGVHWVRQPPGKGLEWLGVIWAGGFTNYNSALKSRLSISKDNSKSQVLLKMTSLQTDDTAMYYCARRGSSYSMDYWGQGTSVTVSS

SEQ ID NO: 43 - protein, 106 aa, Artificial Sequence
  Synthetic
EIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQ

SEQ ID NO: 44 - protein, 121 aa, Artificial Sequence
  Synthetic
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISS

SEQ ID NO: 45 - protein, 112 aa, Artificial Sequence
  Synthetic
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTG

SEQ ID NO: 46 - protein, 662 aa, Artificial Sequence
  Synthetic
EIVLSQSPAITAASLGQKVTITCSASSNVSYIHWYQQRSGTSPRPWIYEISKLASGVPVRFSGSGSGTSYSLTISSMEAEDAAIYYCQQWNYPLITFGSGTKLEIQRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVQSGAEVKKPGASVKVSCKASGFSFTNYYVHWMRQAPGQGLEWMGWISPGSDNTKYNEKFKGRVTMTRDTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 220 aa, Artificial Sequence
  Synthetic
QVQLKESGPGLVAPSQTLSITCTVSGFSLTSYGVHWVRQPPGKGLEWLGVIWAGGFTNYNSALKSRLSISKDNSKSQVLLKMTSLQTDDTAMYYCARRGSSYSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 48 - protein, 219 aa, Artificial Sequence; identical to SEQ ID NO: 36
  Synthetic

SEQ ID NO: 49 - protein, 667 aa, Artificial Sequence
  Synthetic
ELVLTQSPSVSAALGSPAKITCTLSSAHKTDTIDWYQQLQGEAPRYLMQVQSDGSYTKRPGVPDRFSGSSSGADRYLIIPSVQADDEADYYCGADYIGGYVFGGGTQLTVTGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSEVQLVQSGAEVKKPGASVKVSCKASGFSFTNYYVHWMRQAPGQGLEWMGWISPGSDNTKYNEKFKGRVTMTRDTSISTAYMELSRLRSDDTAVYYCARDDYGNYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 224 aa, Artificial Sequence
  Synthetic
QEQLVESGGRLVTPGGSLTLSCKASGFDFSAYYMSWVRQAPGKGLEWIATIYPSSGKTYYATWVNGRFTISSDNAQNTVDLQMNSLTAADRATYFCARDSYADDGALFNIWGPGTLVTISSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 51 - protein, 219 aa, Artificial Sequence; identical to SEQ ID NO: 36
  Synthetic